(19) United States
(12) Patent Application Publication
(10) Pub. No.: US 2026/0048271 A1
(43) Pub. Date: Feb. 19, 2026

(54) PHOTOBIOMODULATION AND ELECTRICAL STIMULATION DEVICES

(71) Applicant: Kineon, Inc., Atlanta, GA (US)

(72) Inventors: Christopher Lee Bohler, Peachtree City, GA (US); Yonatan Ali Rodriguez Arias, Mexico City (MX); Forrest Edward Smith, Atlanta, GA (US); Joseph Patrick Lee, Stoneham, MA (US); Aaron Robby Rogers, Eugene, OR (US); Mary Therese Lee, Stoneham, MA (US)

(21) Appl. No.: 19/298,879

(22) Filed: Aug. 13, 2025

Related U.S. Application Data
(60) Provisional application No. 63/682,732, filed on Aug. 13, 2024.
(60) Provisional application No. 63/736,197, filed on Dec. 19, 2024.

(51) Int. Cl. A61N 5/06; A61N 5/067

(52) U.S. Cl. CPC A61N 5/0613; A61N 2005/0651; A61N 2005/0659; A61N 2005/0663; A61N 5/067

(57) ABSTRACT

The present disclosure relates to diagnostic and therapeutic applications of photobiomodulation and light therapies such as red-light therapy and electrical stimulation devices. Application areas include gut, uterine and other pelvic conditions, back pain, endometriosis, fibromyalgia, etc. The methods and devices disclosed herein are used for delivering targeted therapy using personalized and adjustable treatment protocols which may use features such as real-time monitoring and closed-loop feedback. The devices described herein can be worn discretely to deliver therapy. The present disclosure also includes artificial intelligence and machine learning-based methods for such diagnostic and therapeutic applications.

### CROSS-REFERENCE TO RELATED APPLICATIONS

This application claims the priority benefit of U.S. Provisional Patent Application Ser. No. 63/682,732, filed Aug. 13, 2024, and the priority benefit of U.S. Provisional Patent Application Ser. No. 63/736,197 filed Dec. 19, 2024, each of which are herein incorporated by reference in their entirety.

### TECHNICAL FIELD

This disclosure relates generally to systems and methods for delivering Photobiomodulation (PBM), electrical stimulation, and other therapies to target portions of a body. The disclosed therapies may be used for managing or treating inflammation, aches, pain, and other conditions.

### BACKGROUND

Many people suffer from conditions such as muscle aches and inflammation from acute (e.g., exercise related) and/or chronic causes. Drugs such as painkillers are used to manage aches, pain, inflammation, etc., but have side effects. The medical risks of drug therapy often increase with longer term use. Thus, drugs are often not a viable option in the long term. Exercise, yoga, etc. are often used, but they do not target specific pain or inflammation sites and are often ineffective.

PBM therapy is a non-invasive therapy where light of specific wavelengths is applied to tissues to induce specific biological effects. PBM therapy has been used to produce therapeutic effects such as reduction of inflammation, pain reduction, and promotion of tissue healing.

### SUMMARY

In some aspects, the techniques described herein relate to a system for delivering a photobiomodulation therapy, the system including: a wearable device including: an attachment means for securing to a user or a region of clothing worn by the user, and a first module attachment site; and a first photobiomodulation module adapted to reversibly attach to the first module attachment site.

In some aspects, the techniques described herein relate to a system, wherein the attachment means includes one of: a strap, a cap, a belt, a clip, one or more elements that fully or partially encircle a region of a body of the user, one or more wearable garments, one or more elements that are worn over one or more regions of a torso or extremities of the user, one or more wearable elements that wrap around one or more abdominal organs of the user, one or more elements placed over a head of the user, and one or more elements placed over a neck of the user.

In some aspects, the techniques described herein relate to a system, further including a second module attachment site and a second photobiomodulation module adapted to reversibly attach to the second module attachment site, the second photobiomodulation module being synchronized according to at least one working parameter of the first photobiomodulation module.

In some aspects, the techniques described herein relate to a system, wherein the first photobiomodulation module is adapted to detect attachment of the first photobiomodulation module to the first module attachment site.

In some aspects, the techniques described herein relate to a system, wherein the wearable device is adapted to be secured to the user at one or more of: a head region, a neck region, a chest region, a belly region, a back region, an abdominal region, one or more arm regions, and one or more leg regions.

In some aspects, the techniques described herein relate to a system, further including a charging case adapted for storing and charging the first photobiomodulation module.

In some aspects, the techniques described herein relate to a system, wherein one or more working parameters of the first photobiomodulation module are tunable.

In some aspects, the techniques described herein relate to a system, wherein one or more working parameters of the first photobiomodulation module are programmable using a wireless or a wired connection to the first photobiomodulation module.

In some aspects, the techniques described herein relate to a system, further including a sensor on the first photobiomodulation module.

In some aspects, the techniques described herein relate to a system, wherein the attachment means are adapted to be placed on a side of the region of the clothing opposite to the first photobiomodulation module.

In some aspects, the techniques described herein relate to a computer-implemented method of delivering photobiomodulation therapy to a user, the method including: identifying one or more photobiomodulation modules configured to deliver an individualized photobiomodulation therapy to the user through a wearable device; identifying one or more module attachment sites configured to reversibly couple to the one or more photobiomodulation modules; receiving a connection signal indicating that the one or more photobiomodulation modules are coupled to the one or more module attachment sites; and delivering photobiomodulation using the one or more photobiomodulation modules.

In some aspects, the techniques described herein relate to a method for delivering a photobiomodulation therapy to a user, the method including: programming a first photobiomodulation module to deliver photobiomodulation at one or more working parameters, attaching a first wearable device to a first location on a body of the user, attaching the first photobiomodulation module to a first location of the first wearable device, delivering photobiomodulation through the first photobiomodulation module at the one or more working parameters.

In some aspects, the techniques described herein relate to a method, wherein one or more working parameters include one or more of: an on/off status of a therapy, an emitted wavelength(s), a wavelength distribution and/or bandwidth, a power output, an irradiance, a fluence, a pulse frequency, a pulse width, a duty cycle, a coherence, a beam divergence, a polarization, a spot size, a beam diameter, a beam profile, a delivery mode, an incidence angle, a number and/or type of emitters that are operational, a cooling mechanism(s), a therapy duration, an on/off status of a display, information displayed on a screen, an on/off status of a user interface, a type of information displayed on a user interface, and for one or more sensors in use: an on/off status, a type, working parameters, a sampling frequency, an energy output, and an energy consumption.

In some aspects, the techniques described herein relate to a method, further including adjusting at least one of the one or more working parameters.

In some aspects, the techniques described herein relate to a method, wherein the adjusting of the one or more working parameters is performed by one or more of: a manual process through a user interface, an automatic process performed by the first photobiomodulation module, an automatic process performed by an application loaded on an electronic device in communication with the first photobiomodulation modules or one or more sensors in use during the photobiomodulation therapy, responsive to detecting a magnetic attachment to the first photobiomodulation modules, and using data from the one or more sensors.

In some aspects, the techniques described herein relate to a method, further including attaching the first photobiomodulation module to a second location of the first wearable device.

In some aspects, the techniques described herein relate to a method, further including attaching a second photobiomodulation module to a second location of the first wearable device and delivering photobiomodulation using the second photobiomodulation module.

In some aspects, the techniques described herein relate to a method, wherein at least one working parameter of the second photobiomodulation module is different than a working parameter of the first photobiomodulation module.

In some aspects, the techniques described herein relate to a method, further including attaching a second photobiomodulation module to a first location of a second wearable device and delivering photobiomodulation using the second photobiomodulation module.

In some aspects, the techniques described herein relate to a method, wherein the first photobiomodulation module delivers photobiomodulation to one or more of: a brain region, a skin region, a scalp region, one or more hair follicles, on or more eyes, regions of an oral cavity, a jaw region, a neck region, a spine region, a shoulder region, an elbow region, a wrist region, one or more hands, one or more feet, one or more fingers, a chest region, a heart region, one or more lung regions, a liver region, one or more kidney regions, a bladder regions, a uterus region, a prostate region, a pelvic floor region, a colon region, a rectum region, a perineum region, one or more reproductive organs, one or more skeletal muscles, one or more cardiac muscles, one or more joints, one or more tendons, and one or more bones.

In some aspects, the techniques described herein relate to a method, further including: providing a second photobiomodulation module, wherein the method further includes synchronizing at least one working parameter of the first photobiomodulation module to at least one working parameter of the second photobiomodulation module.

### BRIEF DESCRIPTION OF THE FIGURES

FIG. 1A shows a view of a user wearing an example wearable therapy device.

FIG. 1B shows a side view of a user wearing an example \ wearable therapy device.

FIG. 1C shows a view of a user wearing an example wearable therapy device.

FIG. 1D shows another view of a user wearing an example wearable therapy device.

FIG. 1E shows an example therapy module being attached to an example module attachment site.

FIG. 1F shows an example attachment mechanism used to connect modules to a grid-like structure with multiple attachment sites.

FIG. 1G shows an example storage and charging case for one or more modules described herein.

FIG. 1H shows a perspective side view of an example module including a display.

FIG. 1I show a perspective view of an underside of an example therapy module.

FIG. 1J shows a bottom side view of an example therapy module.

FIG. 1K shows a bottom side view of an example therapy module.

FIG. 1L shows a side view of a subset of the inner electrical components of an example therapy module.

FIG. 1M shows an exploded view of a subset of the components of an example therapy module.

FIG. 1N shows an example power management board.

FIG. 2A shows an example device adapted for delivering therapy to a neck area.

FIG. 2B shows a user wearing an example treatment device around a neck region.

FIG. 2C shows an example device for delivering transcutaneous vagus nerve stimulation (tVNS), therapy to the neck and ear regions.

FIG. 3A shows a flow chart of an example method of delivering adjustable photobiomodulation (PBM) and other therapies to a patient.

FIG. 3B shows a flow chart of an example method of delivering adjustable PBM and other therapies to a patient.

FIG. 4A shows an example wearable device positioned on an upper back region such that the wearable device extends up to one or both shoulders of a patient.

FIG. 4B shows an example wearable device positioned on a lower back region such that the wearable device extends down to a waist of a patient.

FIG. 4C shows an example wearable device positioned on a back of the patient such that the wearable device extends from one or both shoulders of a patient to a waist of the patient.

FIGS. 4D-4E show example side views of a user using an example combination therapy including a light-based therapy and an electrical stimulation-based therapy.

FIGS. 4F-4H show example views of furniture or other household items that contain one or more modules.

FIG. 5A shows a view of pelvic anatomy with example locations of endometriosis that can be targeted using one or more method and/or device embodiments described herein for symptom relief.

FIG. 5B shows a front view of a user wearing an example wearable device for treating a pelvic condition such as endometriosis.

FIG. 5C shows a front view of a user wearing another example wearable device for treating a pelvic condition such as endometriosis.

FIG. 5D shows a front view of a user wearing an example wearable device for treating a uterine or peri-uterine condition.

FIG. 5E shows a side view of a user wearing an example wearable device for treating a pelvic condition such as endometriosis.

FIG. 5F shows an example wearable device in which four modules are placed circumferentially around a torso of a patient.

FIG. 5G shows an example wearable device in which three modules are placed around a uterus of a patient or a peri-uterine region.

FIG. 5H shows an example wearable device in which three modules are placed around a uterus of a patient or a peri-uterine region.

FIG. 5I shows an example wearable device in which a single module is placed to target an abdominal region or organ.

FIG. 5J shows an example wearable device module configured to be worn underneath clothes.

FIG. 5K shows an example wearable device with one or more sensors embedded into clothing.

FIG. 5L shows an example module configured to couple to a connector.

FIG. 6A shows a flow diagram of an example method of delivering a therapy using a system that incorporates sensor feedback and modifies therapy parameters dynamically.

FIG. 6B shows a flow diagram of an example method of delivering a therapy using a system that incorporates sensor and user feedback and modifies therapy parameters dynamically.

FIG. 7A shows a flow diagram of an example method of using machine learning (ML) and artificial intelligence (AI) to deliver therapy.

FIG. 7B shows a flow diagram of an example method employing ML and AI for delivering therapy targeting endometriosis and other pelvic pain conditions.

FIG. 7C shows a flow diagram of an example method employing ML and AI for delivering therapy targeting back pain and related conditions.

FIG. 8A is a diagram showing the interaction of the gut and the brain and some bodily regions where one or more therapies disclosed herein may be delivered.

FIGS. 8B-8F show various embodiments of example devices for delivering therapy to the head.

FIGS. 8G and 8H show two example circumferential locations of PBM modules around the head.

FIGS. 8I-8J show example placement locations of one or more modules relative to a head of a patient.

FIGS. 8K-8L show example placement locations of one or more PBM modules relative to the head of a patient.

FIG. 8M shows a flow chart of an example closed-loop method of delivering a therapy to the head.

FIG. 8N shows a flow chart of another example closed-loop method of delivering a therapy to the head.

FIG. 8O shows a flow chart of an example method of delivering PBM that uses brain biomarker measurements.

FIG. 8P shows an example device that is designed to deliver PBM to one or more head regions having hair.

FIG. 8Q shows an example of a spectral profile of emitted light from one or more PBM modules along with absorption spectra of specific chromophores.

FIG. 9 shows a system diagram of an example computing platform that can be used in any of the embodiments disclosed herein.

### DETAILED DESCRIPTION

The present disclosure will now be described through various embodiments. Even though only a few embodiments have been disclosed, modification of these embodiments and other embodiments may be utilized, without departing from the spirit or scope of the subject matter presented herein. Aspects of the disclosure, as described and illustrated herein, can be arranged, combined, modified, and designed in a variety of different formulations, all of which are explicitly contemplated and form part of this disclosure.

Conventional photobiomodulation (PBM) therapies that exist are limited by ease of use, efficacy, and portability. Further, such conventional devices and methods cannot be easily adjusted and adapted for prescriptions, requests, indicators, physician instructions, and/or other patient-related treatment indication. Such conventional devices utilize a single light or probe function and configuration repeated across a device surface and each of the repeated lights or probes provide the exact same output. The systems described herein provide PBM therapy that is tunable on a per module basis (e.g., each light, group of lights, probe, group of probes, are individually tunable and tunable as a group).

Conventional PBM therapies cannot be easily applied to specific regions of the body such as the back of the abdomen, back of the neck, lower back, etc. For example, many women suffer from severe pain due to uterine cramping, endometriosis, and other conditions. Current PBM therapies cannot be placed consistently and securely on the abdomen and tailored to individual patients. The devices described herein solve the above technical problems with technical solutions. For example, the wearable devices described herein are designed to be positioned accurately at one or more target locations and/or regions on the body. The devices may also be repositioned. The modular design of the devices disclosed herein may be used to deliver therapy at multiple bodily regions where the therapy parameter(s) may vary across the modules. Further, embodiments using closed-loop guidance disclosed herein may be used to tailor the therapy requests and/or prescriptions associated with a user.

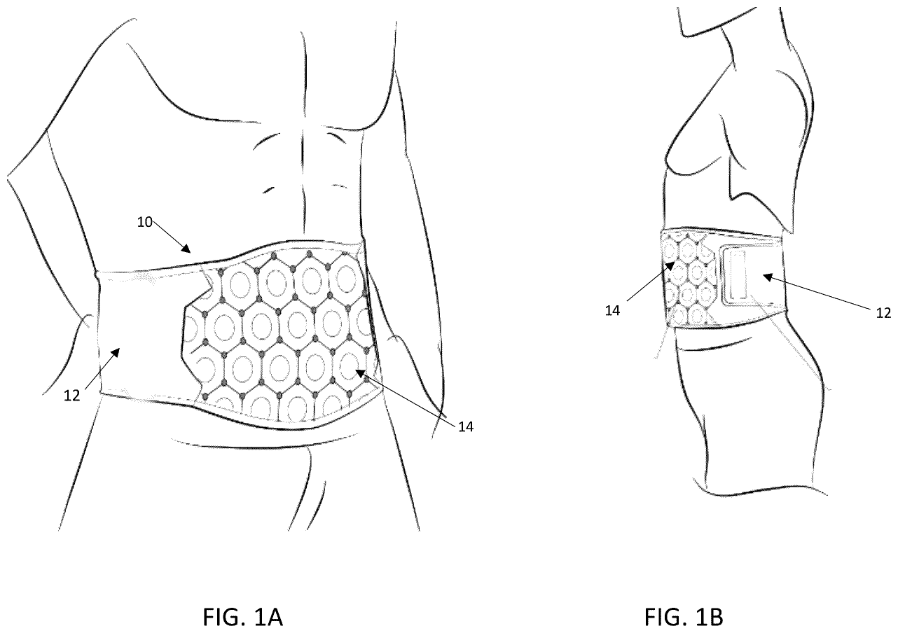

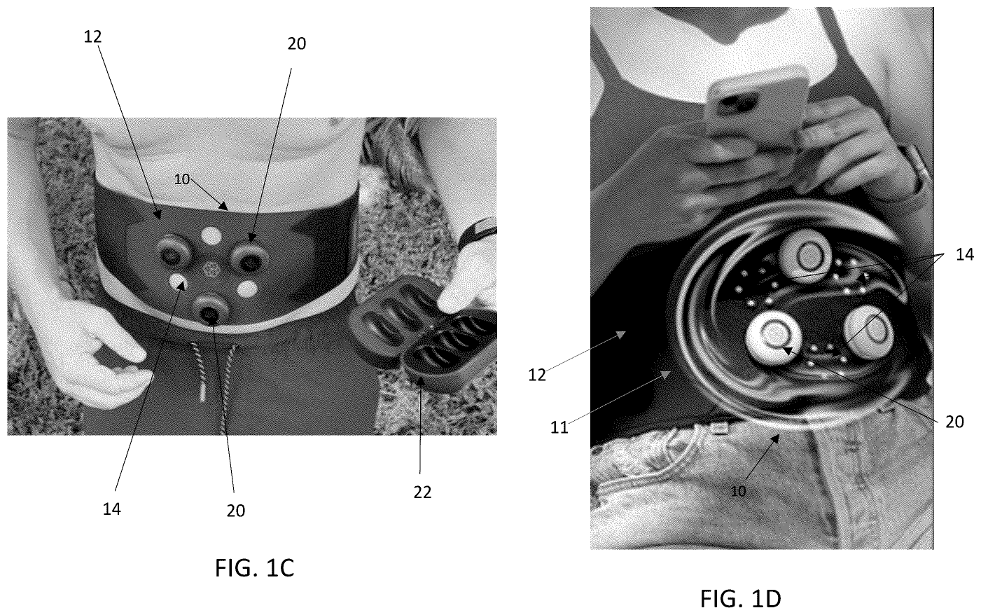

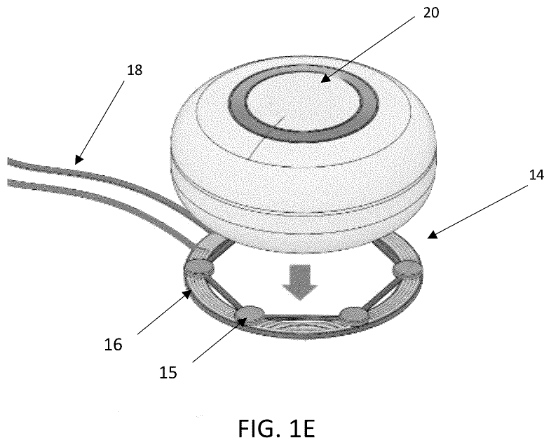

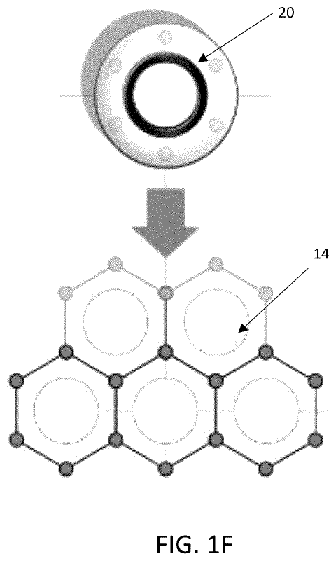

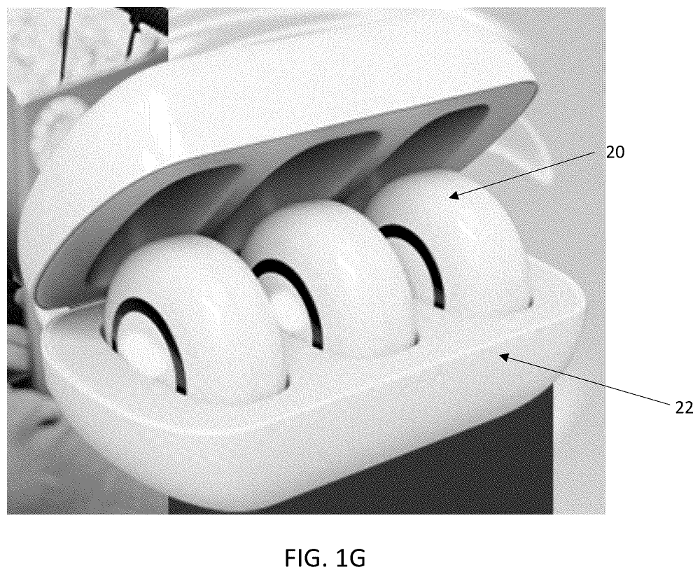

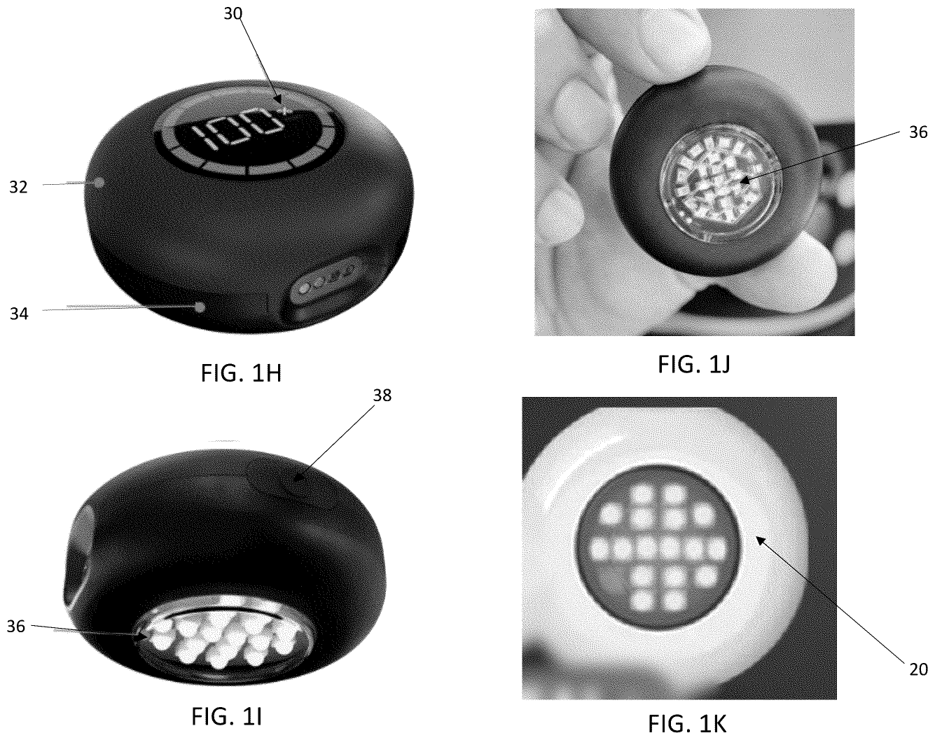

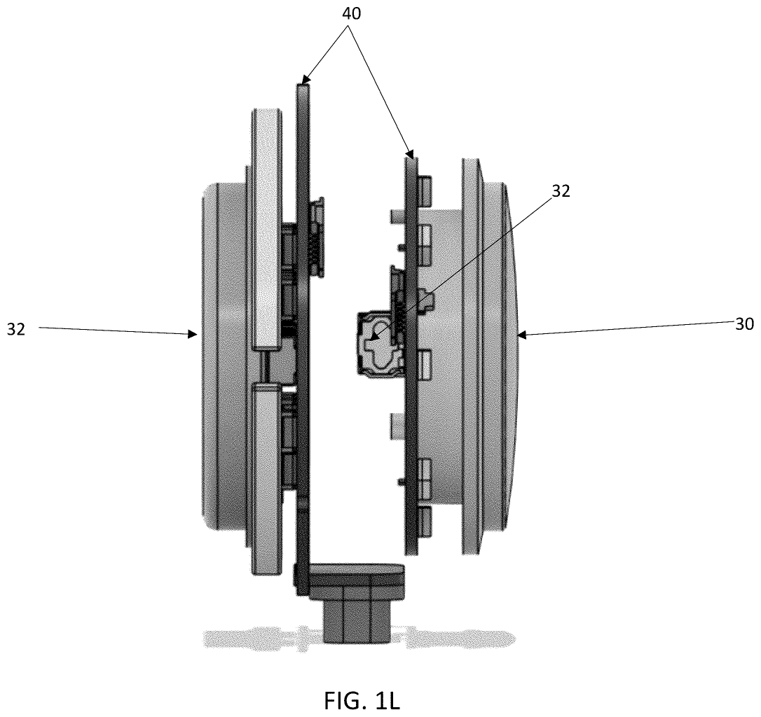

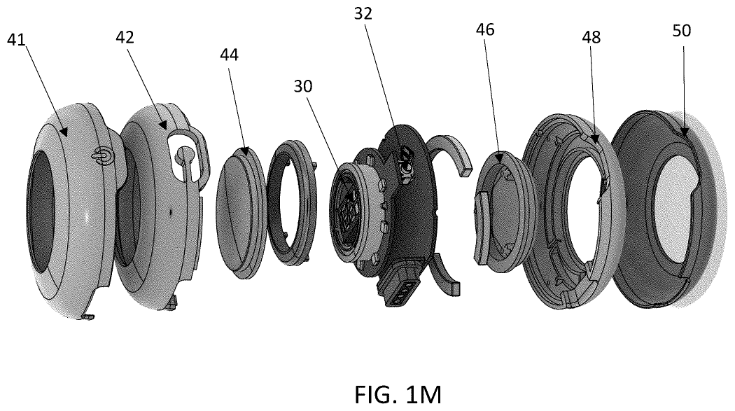

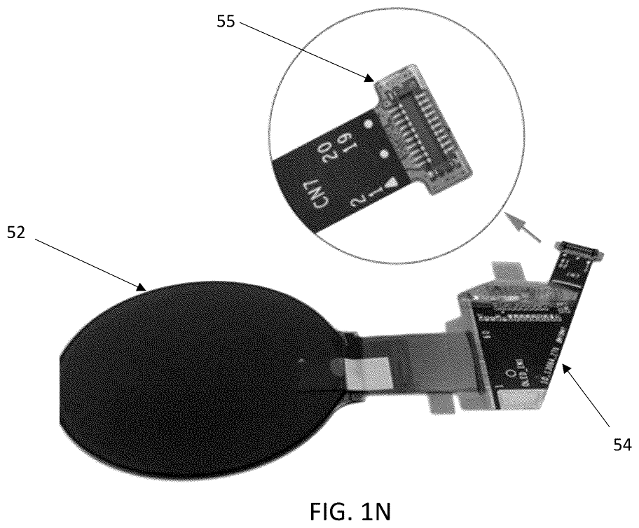

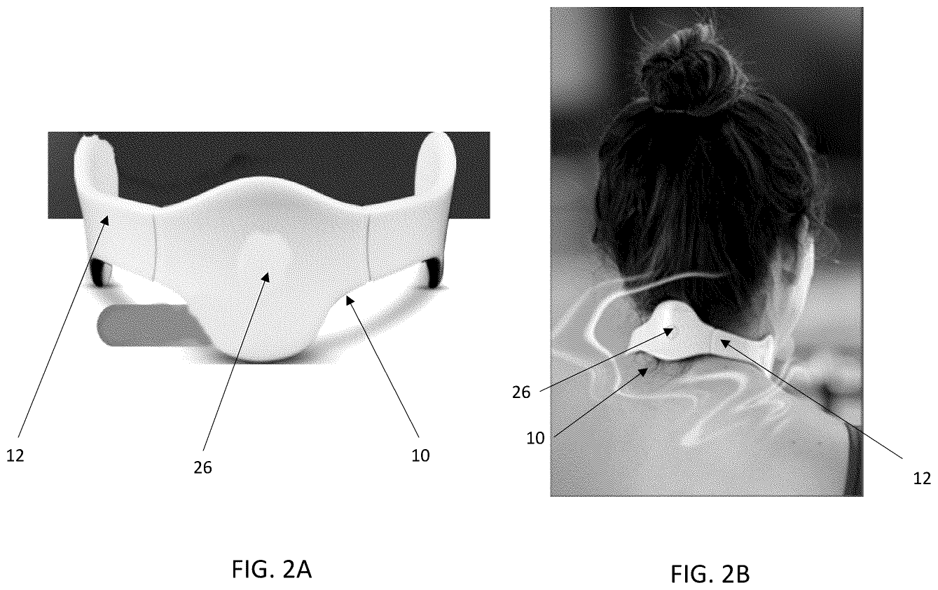

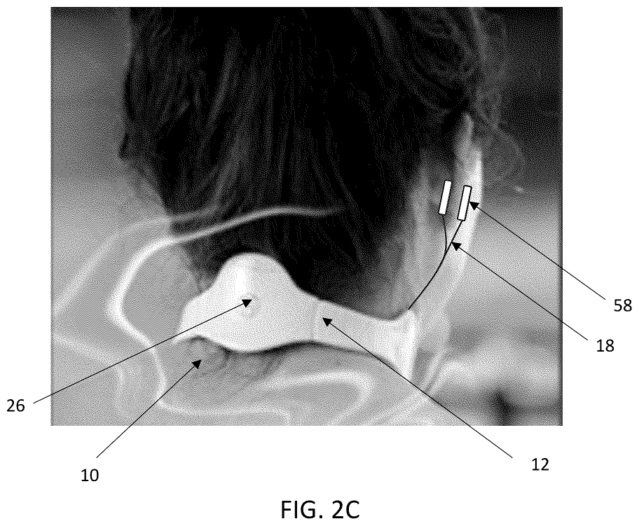

One or more of the devices and methods disclosed herein may be used individually or together (e.g., the embodiment shown in FIG. 1C may be used with the embodiment of FIG. 2B) for various uses; examples of which include, but are not limited to:
(1) Pain Relief. The therapies disclosed herein may be used to generate or cause an analgesic effect. The therapies disclosed herein may be used to reduce pain perception. The therapies disclosed herein may be used to modulate the activity of peripheral nerves, potentially increasing the pain threshold. The therapies disclosed herein may be used to trigger the release of endorphins and other natural analgesic substances that reduce pain signals sent to the brain. Such mechanisms of actions can be used for easing pain due to endometriosis, adenomyosis, and other conditions.
(2) Prompting cell regeneration and repair. PBM triggers cell repair and regeneration primarily through its interaction with the mitochondria, the energy-producing power houses of the cells. Through absorption and modulation of the enzyme cytochrome-c-oxidase (CCO), an increase in the production of adenosine triphosphate (ATP) takes place. With more ATP, cells perform their functions more efficiently, including cell repair and regeneration. This process also stimulates the release of growth factors which play a crucial role in tissue repair and regeneration.
(3) Reducing Inflammation. The therapies disclosed herein may be used to inhibit pro-inflammatory cytokines, which are molecules that promote inflammation. This may be used for reducing back pain since chronic inflammation can contribute to conditions such as sciatica, disc herniations, or general muscular strain. The reduction in inflammatory markers may also help to decrease pain and promote tissue repair. Such anti-inflammatory effects can be used for conditions such as endometriosis, chronic endometritis, etc.
(4) Enhanced Blood Flow and Oxygenation. The therapies disclosed herein may be used to improve blood circulation by promoting vasodilation, or the widening of blood vessels. Enhanced circulation may be used to bring more oxygen and nutrients to an injured area and flush out toxins and waste products, thereby accelerating the tissue healing process. In patients with back pain, improved circulation may be used for faster recovery of strained muscles, tendons, and ligaments.
(5) Post-surgical Recovery. The therapies disclosed herein may be used to accelerate healing after abdominal and/or pelvic surgeries by reducing inflammation, pain, etc.
(6) Improving Fertility. The therapies disclosed herein may be used to improve fertility, for example, by improving blood flow.
(7) Muscle Relaxation. The therapies disclosed herein may be used to relax muscles and reduce spasms. Such spasms may produce back pain and other pain conditions. Relaxation of muscles may be used to improve flexibility and range of motion, thereby providing relief from pain caused by muscle stiffness.

(8) Stimulating a gut-brain connection. The therapies disclosed herein may be used to influence functioning of the gut by one or more of: influence gut microbiome (e.g. by increasing gut microbiome diversity), reduce inflammation in one or more gut regions, and modulate neurotransmitter production. This, in turn, may be used to influence brain functions such as improving mood and/or cognitive functions.
(9) Stimulation of Collagen Production. The therapies disclosed herein may be used to stimulate collagen synthesis, which in turn can be used for improving repair of tissues that may be contributing to pain.
(10) Modulation of the levels of molecules involved in the inflammation process. Examples of such molecules include, but are not limited to: (a) C-reactive protein (CRP): a protein that increases in response to inflammation; (b) cytokines (e.g., IL-6, TNF-α): small proteins released by cells that play a key role in regulating inflammation; (c) prostaglandins: lipid compounds that contribute to inflammation and pain; and (d) nitric oxide (NO): a signaling molecule that can indicate oxidative stress and inflammation.

FIG. 1A shows a view of a user wearing an example wearable therapy device. FIG. 1A shows a wearable therapy device 10 that includes attachment means for securing wearable therapy device to a user or a region of clothing worn by the user. In FIG. 1A, attachment means may include an attachment mechanism 12 to attach to a region of the body. In this embodiment, the attachment mechanism is a strap 12. Example attachment means may include one or more of a strap, a cap, a belt, a clip, one or more elements that fully or partially encircle a region of a body of the user, one or more wearable garments, one or more elements that are worn over one or more regions of a torso or extremities of the user, one or more wearable elements that wrap around one or more abdominal organs of the user, one or more elements placed over a head of the user, and one or more elements placed over a neck of the user. Other example attachments mechanisms are shown in FIGS. 2A-2C (one or more elements that fully or partially encircle a region of the body such as the neck), 4A-4C (one or more wearable garments or elements that are worn over one or more regions of the torso or chest, or belly, or back or extremities such as arms or legs), 5B-5E (one or more wearable elements that wrap around one or more abdominal regions or organs), 8B-8D (one or more elements placed over the head), etc.

For example, strap 12 in this embodiment may secure wearable device 10 around a waist of the user. The strap 12 may be designed to adjust to fit various body sizes. For example, strap 12 can fit sizes S, M, L, XL, XXL, etc. In some embodiments, strap 12 fits waists ranging from 24 inches to 46 inches in circumference. Adjustment of strap 12 may be done by an adjusting mechanism, for example a hook-and-loop fastener, a button securable in a corresponding eyelet, a zipper, a hook securable in a corresponding eyelet, etc. In some embodiments, a portion of strap 12 is located around a narrowest part, or a substantially narrow portion, of a waist when worn by the user. Wearable device 10 may also include a grid type network or array which includes one or more module attachment sites 14 that are designed to be reversibly or permanently attached to one or more modules 20 (shown at least in FIG. 1C and other figures described herein). In some embodiments, one or more modules 20 may be reversibly attached to one or more attachment sites 14. In such embodiments, the one or more modules 20 may be repositioned on other attachment sites 14 of the same wearable device 10 by the user. The one or more modules 20 and/or their treatment or working parameters may be selected and/or tuned and/or programmed and/or determined as described in detail elsewhere herein. Tuning one or more working parameters (examples of which are described later in this specification) is the process of adjusting and/or controlling and/or dynamically optimizing one or more working parameters of one or more modules 20, for example, based on patient-specific factors, treatment goals, feedback data, etc. Several method embodiments using tuning of one or more working parameters are described herein. Several device embodiments which are designed to allow tuning of one or more working parameters are described herein.

Various embodiments may be designed to deliver PBM to one or more organs or regions of the body. Examples of such organs or regions include, but are not limited to one or more: brain region, skin region, scalp region, hair follicles, eyes, regions of an oral cavity, jaw region, neck region, spine region, shoulder region, elbow region, wrist region, hands, feet, fingers, chest region, heart region, lungs, liver region, kidneys, bladder region, uterus region, prostate region, pelvic floor region, colon region, rectum region, perineum region, reproductive organs, skeletal muscles, cardiac muscles, joints, tendons, and bones. PBM may be used for stimulating or modulating peripheral and central nerves'functioning, lymphatic system functioning, and blood vessels'functioning. Using one or more embodiments disclosed herein, PBM may be delivered simultaneously or sequentially to multiple such organs or regions of the body. In some embodiments, PBM therapy may be delivery automatically or manually, or a combination of both automatic and manual delivery.

FIG. 1B shows a side view of a user wearing an example embodiment of a wearable therapy device. FIG. 1B shows a side view of the user and shows strap 12 with an adjustable design to allow wearable device 10 to securely fit users of the device. In FIGS. 1A and 1B, wearable device 10 is shown with no modules 20 attached to demonstrate an embodiment of attachment sites 14 and their arrangement on wearable device 10.

FIG. 1C shows a view of a user wearing an example wearable therapy device 10. In this example, device 10 includes a strap 12 secured around the waist. As shown in FIG. 1C, three modules 20 are shown attached to three attachment sites 14. Device 10 includes a total of six attachment sites 14; three with attached modules 20 and three are free. Thus, up to six modules 20 may be attached. As described elsewhere herein, the location of modules 20 may be changed. FIG. 1C also shows a view of a charging case 22. Charging case 22 may be used to charge and/or store one or more modules 20. In this embodiment, charging case 22 is designed to charge and/or store up to three modules 20. In an alternate embodiment, modules 20 are electrically powered using a battery or other energy storage mechanism located on device 10.

FIG. 1D shows another view of a user wearing an example wearable PBM device 11. In this example, the device 11 is arranged on an abdomen region of the user by the example strap 12. While device 11 is showing using three PBM modules 20, any number of PBM (or other light/laser devices) may be utilized. For example, three additional attachment sites 14 are depicted on device 11.

FIG. 1E shows an example therapy module being attached to an example module attachment site. In the embodiment shown, module 20 is shown being attached to an attachment site 14 using a magnetic attachment mechanism. Attachment site 14 includes a hexagonal arrangement of magnets 15 that are attached to a base plate 16. Although a hexagonal arrangement is shown, one of skill in the art will appreciate that any arrangement of attachment sites is contemplated herein. For example, the attachment sites could be arranged in any polygonal relationship (e.g., oval, hexagon, triangle, octagon, square, etc.). One advantage of a magnetic attachment mechanism is that the user can easily attach or detach modules 20 as desired. Designs of modules 20 disclosed herein are small and easily applied, thus they exert less pressure or force on the skin or bone. This makes them more comfortable and discreet during use. Any of the skin-contacting regions of one or more modules 20 disclosed herein may include concave surface(s) on a skin or body facing surface of the module 20 to better conform to the skin surface. The concave surface(s) may be tailored to fit the anatomical region where one or more modules 20 may be positioned. Multiple connecting wires 18 are connected to base plate 16. These wires 18 may be used to provide electrical energy to module 20. Wires 18 may also be used to transmit data to/from module 20. Even though the embodiment shown in FIG. 1E shows six attachment sites (e.g., magnets 15), alternative embodiments may be designed including one to more than 20 attachment sites. Multiple attachment sites may be arranged to form a honeycomb structure.

Modules 20 disclosed herein may include one or more diodes or other light sources (e.g., lasers) to emit visible (e.g., red) or infrared (IR) light for photobiomodulation therapy. In some embodiments, module 20 includes one or more Vertical Cavity Surface Emitting Lasers (VCSELs). VCSELs or other light sources disclosed herein may be selected based on factors such as high reliability, beam divergence parameters, and temperature-independent wavelength performance. Light (e.g. visible light, infrared light) emitted by one or more light sources 36 may be diffused, diverged, or otherwise modified. In some embodiments, this is performed using one or more optical elements. Examples of such elements include, but are not limited to lenses, light pipes, mirrors, diffusing layers, etc. Such elements may be present inside modules 20 or on the casing or optical windows of modules 20. One or more light sources 36 (e.g. VCSELs) may be designed to emit a wide or divergent beam of light. In some embodiments, the beam divergence angle may range between about 5 degrees to about 30 degrees; about 5 degrees and about 10 degrees; about 10 degrees and about 15 degrees; about 15 degrees and about 20 degrees; about 20 degrees and about 25 degrees; or about 25 degrees and about 30 degrees. Thus, one advantage of the embodiments disclosed herein is that the emitted light covers a larger surface area of the user's body than that of conventional PBM devices. Reflow soldering may be used for attaching surface mount technology (SMT) components such as light sources to a printed circuit board (PCB). VCSELs or other light sources may be applied or used in multi-chip SMT “2016” packages on Copper metalized Aluminum Nitride (AIN) or Copper Silver (CuAg) substrates. In an alternate embodiment, the light source is a light emitting diode (LED). Such LEDs or other light sources may include one or more lenses. Each module 20 may include between a single light source to about 40 light sources. The wavelength of light emitted by one or more light sources herein may range from about 580 nm to about 1,300 nm.

In the embodiments disclosed herein, one or more wavelengths or other characteristics of radiation emitted by one or more modules 20 may be selected based on factors including, but not limited to: depth of penetration of the radiation and chromophore absorption. Suitable light sources may be selected that emit radiation at the desired wavelengths. PBM treatments may be designed such that at least some photons emitted by one or more modules 20 reach the target area. Penetration depths and/or penetration ability of the radiation may be calculated or otherwise estimated based on water absorption, blood absorption, absorption by other chromophores such as melanin.

To illustrate this, a couple of examples of selection of wavelengths emitted by sensor(s) 20 are described below. Other wavelengths or combinations thereof may thus be selected based on the targeted treatment and pathology. Method and device embodiments may be designed such that hemodynamics are modulated and/or measured and/or sensed by one or more sensors located on any of the devices disclosed herein. Examples of hemodynamics-related parameters that may be modulated and/or measured and/or sensed include, but are not limited to: change in hemoglobin-related parameters such as cerebral or other blood flow, blood oxygenation, etc. In such embodiments, wavelengths such as about 660 nm (peak absorption for deoxygenated hemoglobin) and about 940 nm (peak absorption for oxygenated hemoglobin) may be emitted and/or measured. Method and device embodiments may be designed such that cell mitochondrial energy is enhanced to promote overall health and wellness through ATP phosphorylation. In such embodiments, module(s) 20 may emit radiation that matches the broad absorption peaks of cytochrome-c-oxidase (CCO) that are typically between about 760 to about 980nm. In such embodiments, broad-band light sources in such wavelengths may be used for one or more of modulating ATP-based process(es) and monitoring ATP-based process(es). In some embodiments, a broad absorption spectrum of CCO is used to monitor and/or control CCO generation in a bodily region.

In some embodiments, broadband near infrared sensors (bbNIRS) or functional near infrared sensors (fNIRS) may be used to measure one or more parameters disclosed herein. The specific wavelengths at which any of the diagnostic methods herein (e.g. fNIRS, bbNIRS, etc.) may be chosen on the organ and the type of function of the PBM and other therapies. In some embodiments, one or more light emitters (e.g. one or more LEDs, photodiodes, etc.) may act as sensors by converting light to an electrical signal. Closed-loop methods and devices (examples disclosed elsewhere) may be designed using such light emitters.

In some embodiments, LEDs combined with microcontrollers may be used as sensors. Such sensors may measure the discharge time of the LED's inherent capacitance. In some embodiments, LEDs may function as detectors of their own emitted light. Some examples of number and type of diodes that can be used to build modules 20 include but are not limited to: eight vertical-cavity surface-emitting lasers (VCSELs) emitting about 665 nm light, six VCSELs emitting about 808 nm light, four VCSELs emitting about 850 nm light, six VCSELs emitting about 940 nm light, six VCSELs emitting about 1060 nm light, and three VCSELs emitting about 665 nm light in combination with three VCSELs emitting about 1060 nm light.

Light sources such as VCSELs may be made of III-V semiconductor materials, examples of which include, but are not limited to: AlInGaP, InGaAsP and InGaAs. Each module 20 may include multiple types of light and other energy (e.g., mechanical vibrations or other mechanical energy, thermal energy, audible sound(s), ultrasound, infrasound, etc.) sources to achieve a combined and/or synergistic action of the energies emitted by the multiple energy sources. Any of the modules 20 described herein may simultaneously and/or sequentially target more superficial structures (e.g., subcutaneous structures, abdominal muscles, etc.) with a lower wavelength radiation and deeper structures (e.g., pelvic organs, thoracic organs, etc.) with a higher wavelength radiation. For example, module 20 embodiments may target superficial structures with a red light and deeper structures with infrared light. The type of light emitted by one or more modules 20 disclosed herein may be modulated or adjusted during or before a treatment. For example, the wavelength distribution of one or more modules 20 may be changed by changing the output of one or more light sources 36. Such modulation may be performed through AI-based methods, examples of which are disclosed elsewhere herein.

One or more design parameters (e.g., number, type, location, orientation, etc.) of one or more light or other energy sources may be determined by one or more considerations, examples of which include, but are not limited to: desired location of energy delivery, desired treatment outcome(s), optical output power to deliver a predefined dose for a predefined therapy session time, optical radiation output pattern to fill an optical aperture of module 20 without creating significant hotspots (region of over-concentrated light intensity), series or series-parallel electrical characteristics to be compatible with an available input string voltage, and energy penetration depth. For example, design parameters of one or more light sources may be chosen to achieve a balance between penetration depth and creation of hot spots. Such hotspots may cause localized undesired thermal effects and create undesired treatment gradients.

One or more modules 20 may be designed such that the power output ranges from about 50 mW to about 200 mW. One or more devices such as light sources and modules 20 disclosed herein may be battery powered. In one such example, such devices may be powered with an about 300+/−100 mA-Hrs. Lithium source. Modules 20 may include one or more rechargeable batteries. In some embodiments, module 20 includes a RJD3048HPPV30M, 3.7V 300 mAh lithium-ion coin cell battery. In some embodiments, the combined optical output power for all VCSELs in a module 20 is about 100 mW+/−30 mW. The output beam angles of light sources such as VCSELs may range between about 10 degrees to about 35 degrees. One or more light sources may be operated at about 20% to about 100% duty cycles. Optical windows may be provided in modules 20 to allow light emitted by light source(s) to pass through. A size of one or more optical output apertures may range from about 0.2 cm² to about 2.5 cm². Modules 20 and other components of wearable device 10 may be designed to have a “zero” gap between an output aperture and the skin or clothing surface.

The time of energy delivery during a therapy session may range from about 30 seconds to about four hours. In some embodiments, one or more devices disclosed herein may be designed to allow users to select and run therapy sessions of, for example, about 5, about 10, about 15, about 20 minutes. Such limits may be controlled by device firmware.

The light sources may also be controlled in waveform by use of “pulse-width modulation in which the frequency is varied from about 10 Hz to about 40 Hz at various duty cycles ranging from about 10% to about 50%.

The total irradiance from the light sources (e.g., VCSELs) may range from about 10 mW/cm² to about 200 mW/cm². In some embodiments, the total irradiance from the light sources (e.g., VCSELs) is about 80 mW/cm². Some specific examples of the irradiance level and the corresponding wavelength that can be generated by one or more modules 20 include but are not limited to: about 10 mW/cm² at about 665 nm, about 25 mW/cm² at about 808 nm, about 25 mW/cm² at about 940 nm, and about 20 mW/cm² at about 1060 nm. In some embodiments (e.g. embodiments designed to deliver PBM to the gut), modules 20 deliver light at wavelengths of about 808 nm, about 940 nm, and/or about 1064 nm. Each module 20 may include multiple types of light and other energy sources to achieve a single action or a combined action of the energies emitted by the multiple energy sources.

The fluence from one or more modules 20 disclosed herein may range from about 2 J/cm² to about 60 J/cm² for a therapy time ranging from about 5 minutes to about 20 minutes. Some specific examples of the fluence and the corresponding therapy time that can be generated by one or more modules 20 include but are not limited to: about 20 J/cm² at about 5 minutes therapy time, about 40 J/cm² at about 10 minutes therapy time, and about 60 J/cm² at about 15 minutes therapy time.

One or more modules 20 or other regions of device 10 disclosed herein may include software and/or hardware features for one or more of: user tracking, visual guide for placement over target tissue, feedback loop(s) for optimum usage/dosing, habit-driving mechanisms, usage pattern recognition (e.g., based on machine-leaning based applications, based on diurnal cycle, based on eating habits, based on timing, etc.), etc. One or more devices 10 or components thereof may include sensors and other hardware, data storage mechanisms, integrated software solutions/platforms, telemedicine functionality (e.g., to allow data download to a medical practitioner's site), and visual displays. One or more device 10 or components thereof may include sensors that are used for purposes such as confirming proper placement on the patient's body, obtaining biometric feedback, etc.

One or more modules 20 or other regions of device 10 disclosed herein may include a button or other mechanism to select/change an application. One or more modules 20 may be self-contained modules that may or may not utilize an external physical connection (e.g. a wire, a cord, etc.) during use. This allows the user to wear such modules 20 without their movement being restricted by connections such as wires or cords. Such self-contained modules 20 may utilize batteries or other non-corded energy sources, PBM emitting elements, and control interfaces, etc.

FIG. 1F shows an example attachment mechanism used to connect modules to a grid-like structure with multiple attachment sites 14. Modules 20 can be attached in series or series-parallel electrical characteristics to match the available input string voltage requirements. Modules 20 may be selected and attached based on the dosing requirements and desired optical radiation output pattern. In some embodiments, a disease or target location is determined. The target location is them mapped to a wearable grid. One or more modules 20 are connected to desired locations(s) on the grid to deliver therapy to the patient's body.

FIG. 1G shows an example storage and charging case for one or more modules described herein. In the embodiment shown, up to three modules 20 may be stored in case 22. Case 22 may include a rechargeable battery or other energy storage mechanism to charge one or more modules 20 even while traveling (e.g., the case may be portable). Case 22 may include a charge management system that charges rechargeable battery or other energy storage mechanism and modules 20 together. Case 22 may include a programming system that may be used to select or program and/or tune one or more working parameters of one or more modules 20.

FIG. 1H shows a perspective side view of an example module including a display. For example, FIG. 1H depicts a side view of an embodiment of a module 20 including a display 30 (e.g., touchscreen, OLED touchscreen). Display 30 may be a capacitive touch interface and may display parameters such as time, therapy type, tissue/selection area, battery percentage, time left, etc. In this embodiment, module 20 is battery powered with an about 300 mA-Hrs. Lithium source. In this embodiment, the combined optical output power for all VCSELs in module 20 is about 100 mW. Module 20 is made of III-V semiconductor materials (InGaAsP, InGaAs). Module 20 may be firmware controlled for user-choice run-time sessions e.g., about 5, about 10, about 15 minutes. Display 30 can provide additional information related to battery level indication and session run time countdown functionality. Display 30 also includes a Bluetooth Low Energy (BLE) solution based on the Atmosic ATM3330 chipset which allows for the module 20 to have a low energy operation for wake-up and wireless connectivity functionality. Module 20 includes wake-up, module and dock synchronizations, and APP connectivity. Upper casing 32 and lower casing 34 provide a clam-shell housing made of, for example a plastic (e.g., ABS+PC plastic). Module 30 includes a pogo pin pass-through for charging dock connectivity.

FIGS. 1I-1K show views of the underside of example therapy modules. Module 20 includes multiple light sources 36 which provide the PBM therapy through localized red and near infrared light. Such therapy may be applied to the gut area for improving gut health, improving gut biome, and other outcomes. In some embodiments, module 20 includes at least two types of light sources 36, with each light source 36 emitting light at two different wavelengths.

FIG. 1L shows a side view of a subset of the inner electrical components of an example therapy module. In some embodiments, a module 20 may include two printed circuit boards (PCBs) 40 enclosing a rechargeable lithium (e.g., coin cell) battery. In this embodiment, the primary board may contain most of the electronic components. Examples of such components include, but are not limited to: microcontroller, accelerometer, power supplies, VCSEL drivers, VCSELs 36, power switch 32, buzzer and display 30.

FIG. 1M shows an exploded view of some components of an example therapy module. FIG. 1M shows the layout and position of various components of the module 20. Components of module 20 include outer case 41, inner case 42, display cover 44 (to protect display 30), light source cover 46 (a protective layer to protect multiple light producing modules 36), inner base covering 48, and outer base covering 50.

FIG. 1N shows an embodiment of a power management board. Power management board 54 includes a battery board, an indicator, a backup infrared (IR) remote receiver, and an IR remote transmitter 55.

FIG. 2A shows an example device adapted for delivering therapy to the neck area and/or region. Such devices may deliver transcutaneous vagus nerve stimulation (tVNS), such that electrical currents are applied through skin surface electrodes. tVNS may be used to elicit hypo or hyperactivation in various regions of the brain associated with anxiety and mood regulation. These electrodes may target the cervical branch of the vagus nerve in the neck as shown in the embodiments of FIGS. 2A-2B. Device 10 includes a winged like structure support 12 that allows the user to place and secure device 10 around the neck. In this embodiment, device 10 may deliver therapy such as Vagus Nerve Stimulation, PBM, cervical Transcutaneous Electrical Nerve Stimulation (TENS), etc. and their combinations through a treatment module or treatment element 26 (not shown) located on a module holder 28. One or more therapies on device 10 may be combined with sensing or measurement functions (e.g., measuring vagus nerve activity, auricular EEG or an alternate sensing modality). The position of device 10 on the user may be optimized for delivering optimal therapy using feedback mechanism(s). Examples of such mechanisms include, but are not limited to: sensor-based feedback, user feedback, etc.

FIG. 2B shows a user wearing an example treatment device around a neck region. For example, FIG. 2B illustrates a person wearing an embodiment of a treatment device around their neck. In this embodiment, support 12 may include one or more additional modules 20 or treatment elements 26. In this embodiment, waveforms and/or stimulation levels may be adjustable. Device 10 may include features such as closed loop operation, monitoring of meditation and other activities, energy harvesting that harvests energy from one or more of: the user's body, motion of the user, kinetic energy of motion, radiofrequency signals, etc.

Embodiments such as those shown in FIGS. 2A-2C can be used for purposes including, but not limited to: improving and monitoring sleep pattern(s), pain reduction, stress relief, increasing parasympathetic activity, reducing stress levels, monitoring and managing heart rate and Heart Rate Variability (HRV), Mental Health Management (e.g., insomnia/depression/anxiety control), giving stress indicator(s), providing feedback about any of the measurements listed herein, enhancing sports performance (e.g., through improved mental focus, through relaxation training, etc.), improving public speaking and communication (e.g., through performance training), and relaxation training. Stress relief may be used during periods of heavy/intense training, high level performance training, public speaking, etc. to improve the user's performance. Specific waveforms and/or stimulation levels may be used for any of these applications. Specific waveforms and/or stimulation levels tailored for a specific application may be selected through a software and/or hardware user interface. The sensing and electrical current delivery may be performed through a circuit. In some embodiments, separation of sensing signals and therapy signals is performed electronically. The sensing and electrical current delivery may be performed through two circuits—a first circuit for receiving/sensing electrical signals and a second circuit for transmitting therapeutic electrical currents. Any of the embodiments disclosed herein may include one or more sensors to monitor physiological signals such as heart rate, heart rate variability (HRV), respiration parameters, EEG or other electrical sensing, etc. Input from such sensors or other inputs may be used to deliver TENS or other therapies in open or closed-loop modes. Input from such sensors may be used to provide feedback to the user and/or adjust one or more therapy parameters. In one such embodiment, auricular EEG or other sensing modality is combined with a therapeutic modality such as tVNS or TENS. Device 10 may include software and hardware for performing signal comparative analysis using cervical signals. The cervical signals may be correlated to brain EEG signals in a “brain EEG library” that is stored in device 10. The comparative analysis may be used to determine one or more dosing parameters. The “brain EEG library” may store limited data to allow a less complex and reduced dataset to be used in device 10.

In some embodiments, operating parameters of device 10 may be adjustable. Device 10 may deliver TENS therapy where the pulse amplitude varies from about 5 to about 24 V at less than about 3 mA, with about 2 to about 10 pulses per burst at about 50% duty cycle, burst duration of about 200 to about 2000 microseconds at a burst frequency of about 10 to about 50 Hz. Device 10 may allow user to select therapy times e.g., between about 2 to about 20 minutes. Device 10 may include a battery with a runtime above about one hour. In some embodiments, device 10 includes a microprocessor controller box that delivers current(s) to one or more skin contact electrodes. Any of the electrodes disclosed herein may be “gel-free” electrodes. Such electrodes are designed to adhere to skin without using gel or tape. Such electrodes may be made from materials, including but not limited to: carbon, cotton-backed cloth, and soft non-woven backing. In some embodiments, device 10 delivers a waveform in a bipolar asymmetric rectangular shape. Any of the characteristics of the electrical output of one or more devices 10 disclosed herein may be programmable or selectable or tunable. Examples of such characteristics include, but are not limited to: on/off status, frequency, voltage, waveform, etc.

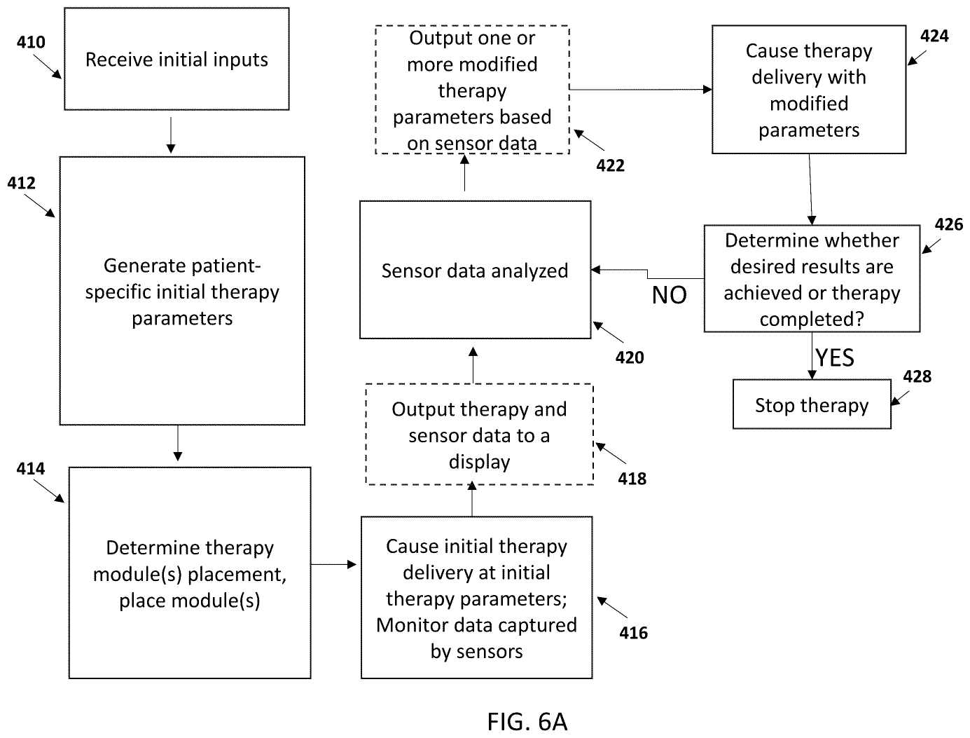

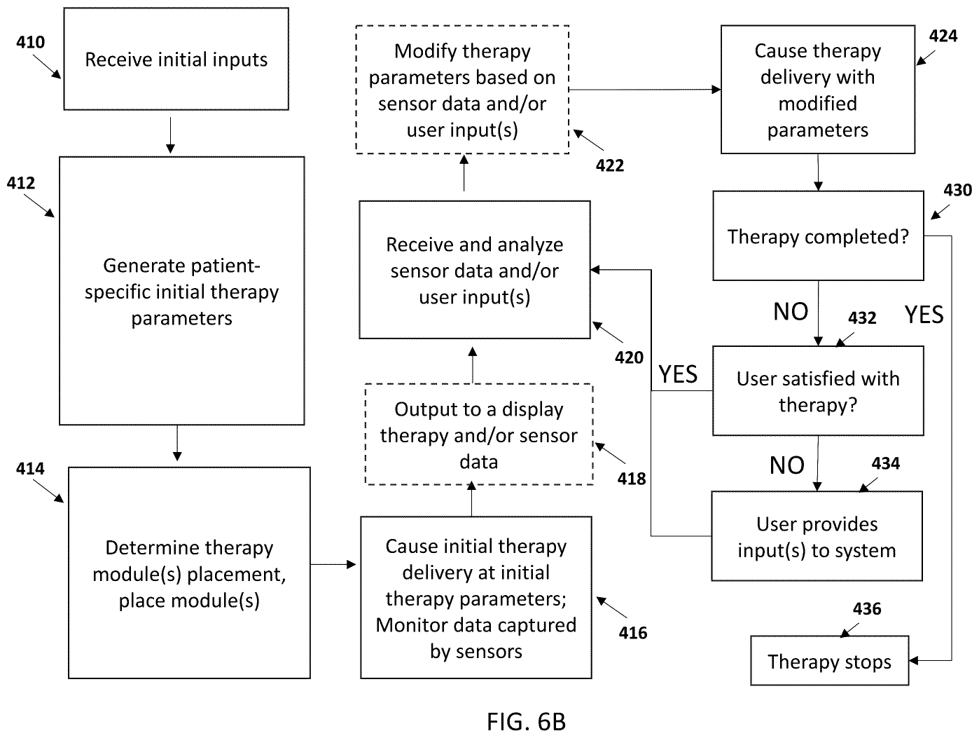

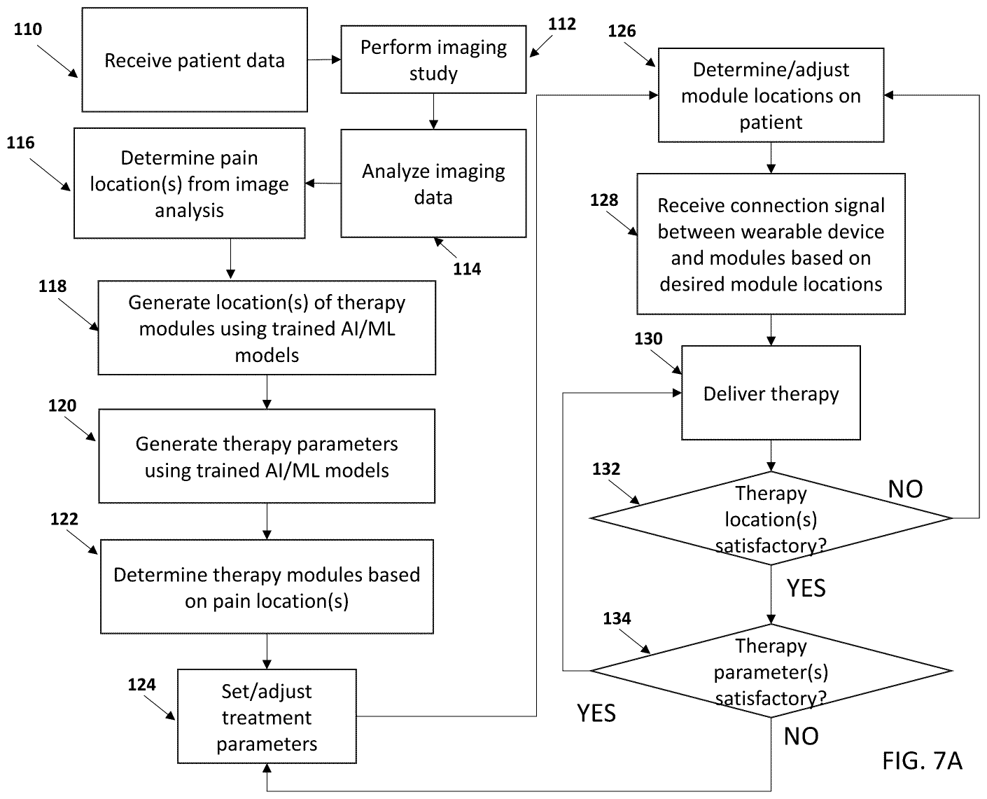

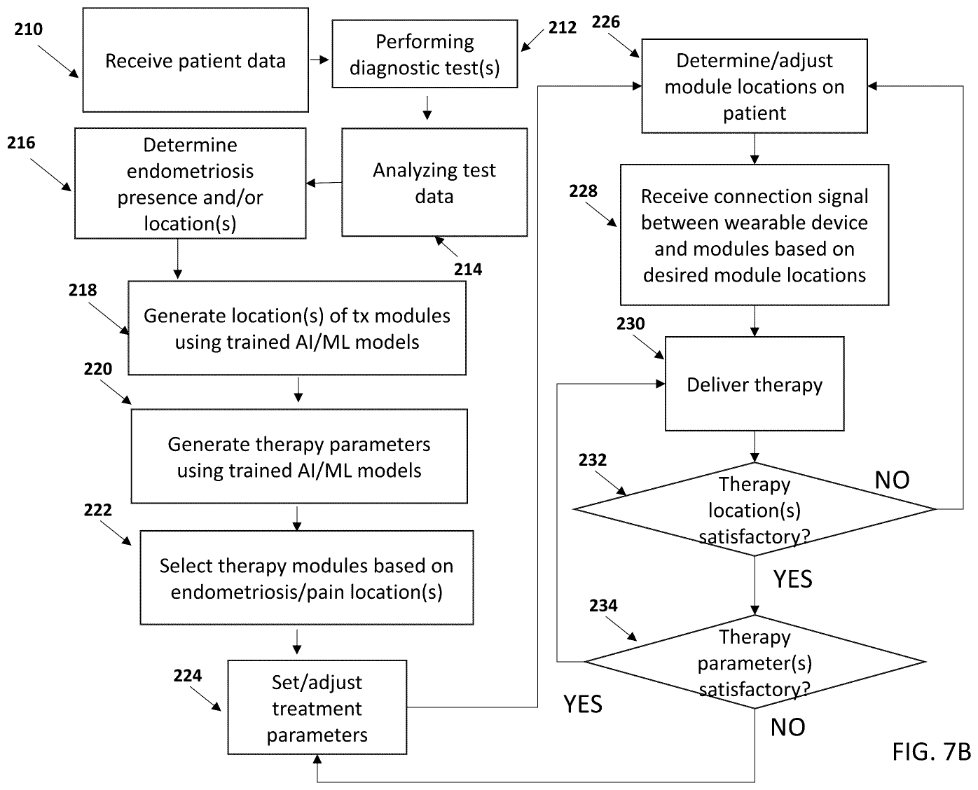

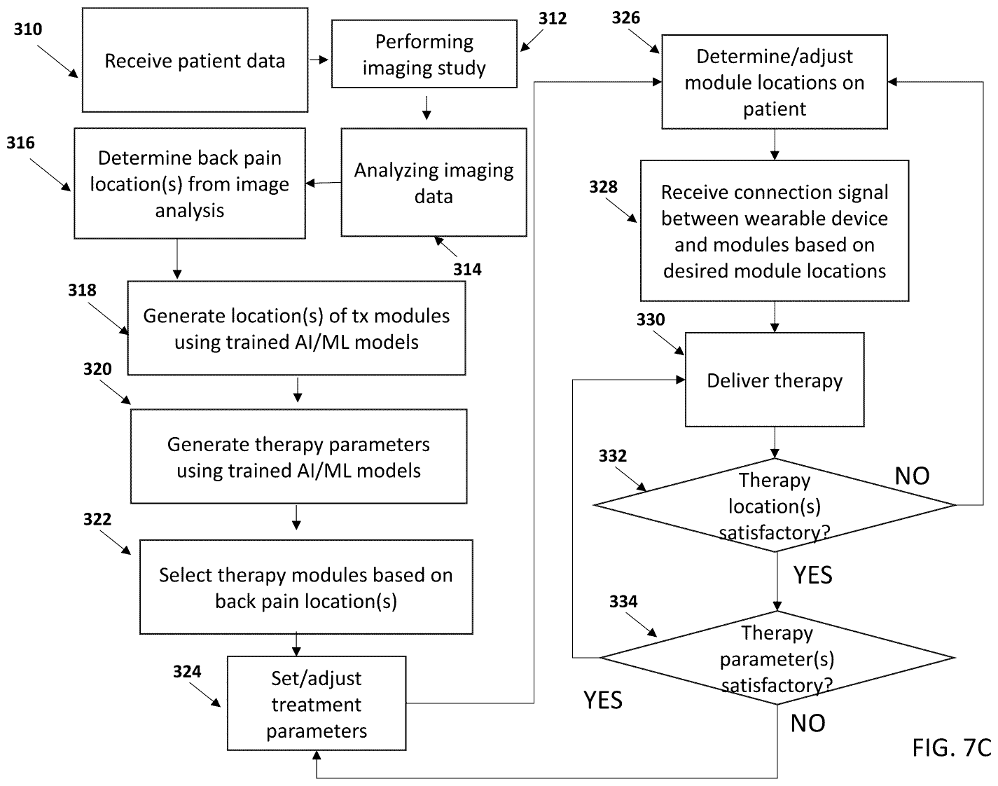

In any embodiment disclosed herein, the user may self-adjust one or more therapy parameters such as the energy dose. In some embodiments, the controller may activate one or more modules of the device 10 with a lower electrical output (e.g., lower current, lower power level, lower dose, lower therapy time, lower duty cycle, etc.), for example based on a received input. The microcontroller may cause an increase in the electrical output until a desired effect is achieved. The microcontroller may continue to increase the electrical output until the therapy is uncomfortable. The microcontroller may cause a lowering in the electrical output until optimal therapy is achieved. AI/ML-based embodiments disclosed herein (e.g., embodiments shown in FIGS. 7A-7C), method embodiments shown in FIGS. 6A-6B, etc. may be used to guide and/or assist the user during this process to achieve optimal therapy in a semi-automated way. One or more methods described herein (e.g., ML-based methods) may be used to automate one or more steps of a process of finding optimal electrode position(s) and therapy parameters.

FIG. 2C shows an example device for delivering transcutaneous vagus nerve stimulation (tVNS), therapy to the neck and ear regions. The embodiment shown uses electrical currents delivered through surface electrodes at select locations to target the auricular branch of the vagus nerve and the cervical branch of the vagus nerve in the neck. Electrodes 58 may be designed to be placed or attached to one or more ear regions (e.g., one or more of: ear lobes, antihelix, tragus, cymba concha, and cavum concha, etc.) or skin regions around the ears. Electrodes 58 are connected to the neck portion of the device through one or more wires 18 and/or tethers. Electrodes 58 may be attached to such regions using a variety of mechanisms like adhesive layers, clips, bands, spring-loaded mechanisms, etc. In some embodiments, one or more electrodes 58 are attached to skin using self-adhesive pads including a conductive solution.

Although FIG. 2C shows an example device for delivering combined therapy to the neck and ear regions, such combined therapy may be delivered through two separate devices. In such embodiments, a first device may be used to deliver tVNS therapy to at least one neck area or region and a second device may be used to deliver tVNS therapy to at least one ear area or region.

One method of using the embodiments shown in FIGS. 2A-2C includes three main steps: positioning one or more device 10 components, obtaining feedback about the placement and/or an initial therapy, and dose adjustment. The step of positioning device 10 includes finding the optimal position of one or more components of device 10 such as electrodes 58, strap 12, energy delivery elements, etc. Device 10 components may be positioned using the position of the vagus nerve as a guide. In some embodiments, a sensor or electrode is used to find the position of the vagus nerve by measuring the amplitude of the signal received from the vagus nerve. One or more portions of device 10 are moved over the skin until a sufficiently large signal amplitude measured that indicates that the sensors/electrodes are positioned over the vagus nerve. Such measurement-based methods may be used along with visual indicators (e.g., locations of one or more neck regions, locations of one or more vertebrae, locations of one or more skull regions, etc.) to guide the placement of one or more device 20 regions. In some embodiments, an initial therapy is delivered and feedback on the effect of the initial therapy is obtained. Such feedback may include one or more of: user's feelings, electrical response to the initial therapy, EEG signals, ECG signals, signal clarity, etc. This feedback may be used to guide the placement of one or more device 10 components.

In some embodiments, a user wears device 10 shown in FIGS. 2A-2C before going to sleep. The user may keep the device 10 attached for a desired time and use device 10 to deliver one or more therapies. Thereafter, the user removes device 10 just before going to sleep. This method may be used to improve one or more sleep parameters, including, but not limited to: sleep duration, sleep quality, sleep apnea, rapid eye movement (REM) sleep, etc.

In some embodiments, specific medication(s) are administered to the user. One or more method embodiments disclosed herein may be used for one or more of: monitoring the effect(s) of the medications, enhancing the effect of the medications, etc. In some embodiments, sleep aids are administered to the user. One or more method embodiments disclosed herein are then used for one or more of: monitoring the effect(s) of the sleep aids on one or more sleep parameters, enhancing the effect of the sleep aids, etc. In some embodiments, one or more anxiolytics are administered to the user. One or more method embodiments disclosed herein are then used for one or more of: monitoring the effect(s) of the anxiolytics, enhancing the anxiolytic effect(s) of the anxiolytics, etc.

The embodiments shown in FIGS. 2A-2C may include one or more mechanically adjustable components e.g., adjustable straps 12, adjustable locations of one or more electrodes 58, etc. to mechanically adjust to various neck sizes, ear sizes, ear locations, etc.

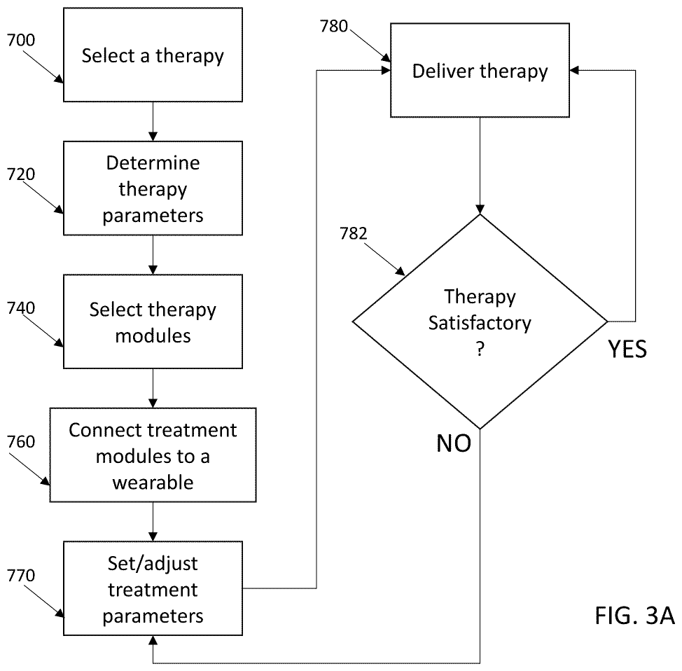

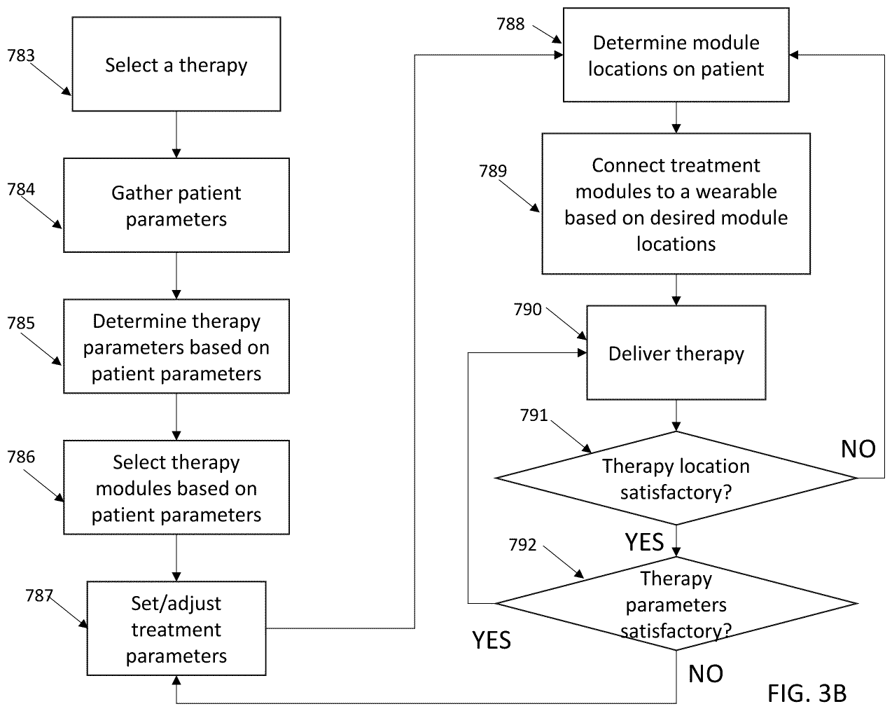

FIG. 3A shows a flow chart of an example method of delivering adjustable PBM and other therapies to a patient. At step 700, a therapy is selected for the patient. Examples of therapies are disclosed in this disclosure. At step 720, one or more therapy parameters are determined. Examples of therapy parameters are disclosed in this specification. They include, but are not limited to: location(s) of energy delivery, energy delivery type, energy delivery parameters, adjustment(s) on one or more modules 20, etc. At step 740, one or more therapy modules 20 are selected. One or more therapy module(s) 20 may be selected based on one or more considerations; examples of which include, but are not limited to: location(s) of the therapies, type of therapy to be delivered, therapy parameters, etc. At step 760, one or more modules 20 are connected to a wearable device 10. At step 770, desired treatment parameters are set or adjusted. This may be done by programming or otherwise setting such treatment parameters on one or more modules 20 or devices controlling one or more modules 20. In any of the embodiments described herein, one or more working parameters (e.g. therapy parameters, displayed data, sensor(s) activity, etc.) may be programmed or selected for a particular application. In some embodiments, multiple modules 20 may be attached to a single wearable device 10, where at least two modules 20 deliver PBM that differs in at least one working parameter (pulse width, power level, wavelength, etc.). In some embodiments, multiple modules 20 may be attached to a single wearable device 10, where at least two modules 20 have the same hardware configuration, but are programmed differently to deliver PBM that differs in at least one working parameter. In some embodiments, at least one working parameter may be synchronized between a first PBM module 20 and a second PBM module on the same device 10. For example, a pulse width, power level, wavelength etc. may be synchronized across two or more PBM modules 20.

One or more computational steps such as programming, selecting parameters, tuning parameters, adjusting working parameters, etc. may be performed by a user through a user interface (e.g. capacitive touch screen 52), automatically by one or more photobiomodulation modules 20, automatically by an application loaded on an electronic device (e.g. a mobile phone, a laptop, a personal computer, an electronic wearable, etc.) in communication with one or more modules 20 and/or one or more sensors 56. In some embodiments, the user may alter one or more working parameters e.g. fluence, therapy time, etc. using an application loaded on an electronic device in communication with one or more modules 20 and/or one or more sensors 56. Examples of such working parameters include, but are not limited to:

Therapy parameters: e.g. on/off status, emitted wavelength(s), wavelength distribution and/or bandwidth, power output, irradiance, fluence, pulse frequency, pulse width, duty cycle, coherence, beam divergence, polarization, spot size, beam diameter, beam profile, delivery mode, incidence angle, number and/or type of emitters (LEDs, lasers, etc.) that are operational, cooling mechanism(s), therapy duration, etc.

Display parameters: e.g. on/off status, information displayed on a screen (e.g. LED screen, touchscreen, etc.) on a module 20, etc.

User Interface parameters: on/off status, type of information displayed on the user interface on module 20, wearable device 10, an electronic device leaded with a control/programming application, etc., and

Sensor parameters: on/off status of one or more sensors, type of sensors in use, working parameters (e.g. sensitivity) of sensors, sampling frequency, energy output, energy consumption, etc.

In some embodiments, adjusting of the one or more working parameters is performed by one or more of a manual process through a user interface on display 80, an automatic process performed by one or more PBM module 20, an automatic process performed by an application (e.g., apps 79) loaded on an electronic device in communication with one or more PBM modules and/or one or more sensors in use during the PBM therapy, and/or may be performed responsive to detecting a magnetic attachment to one or more PBM modules to device 10 using data from the one or more sensors 56.

In some embodiments, modules 20 are designed to emit multiple wavelengths of light. For example, modules 20 may include multiple light emitting elements (examples disclosed elsewhere herein) that emit light at multiple wavelengths. emitted wavelength(s), wavelength distribution, etc. of such modules 20 may be selected or programmed based on one or more factors; examples of which include, but are not limited to: a location of module 20, a desired effect of the action of module 20, a type of pathology being treated or addressed, a type of wearable device 10 and/or which, if any, attachments 90 are connected to module 20, etc.

In some embodiments, modules 20 include multiple sensors 56. For example, modules 20 may include multiple types of sensors 56 that may perform individual sensing functions. The type and/or number of sensors 56 that are operational may be selected or programmed based on one or more factors. Examples of such factors may include, but are not limited to: a location of module 20, a desired effect of the action of module 20, a type of pathology being treated or addressed, a type of wearable device 10 or attachments 90 connected to module 20, etc. Examples of sensors 56 may include, but are not limited to: blood flow sensors, skin temperature sensors, spectroscopy-based sensors, time of flight-based sensors, temperature sensors, impedance sensors, moisture sensors, motion or orientation-detecting sensors (e.g., gyroscopes, accelerometers, or a combination thereof) and electrical sensors. In some embodiments, module 20 or other device portions may comprise multiple sensors 56. Specific sensors 56 may be turned on and used during a treatment based on predefined treatment requirements and/or sensor thresholds and/or user settings.

In some embodiments, a first wearable device 10 may be placed on a first location and a second wearable device 10 may be placed on a second location. At least one module 20 may be programmed to deliver customized PBM to the first location. The module 20 may be attached to first wearable device 10. A second module may be programmed to deliver customized PBM to the second location. The second module may be attached to a second wearable device 10, In some embodiments, the first and the second modules may have the same hardware configuration, but may be programmed differently to deliver PBM therapy that differs in at least one parameter. Examples of such parameters are disclosed herein.

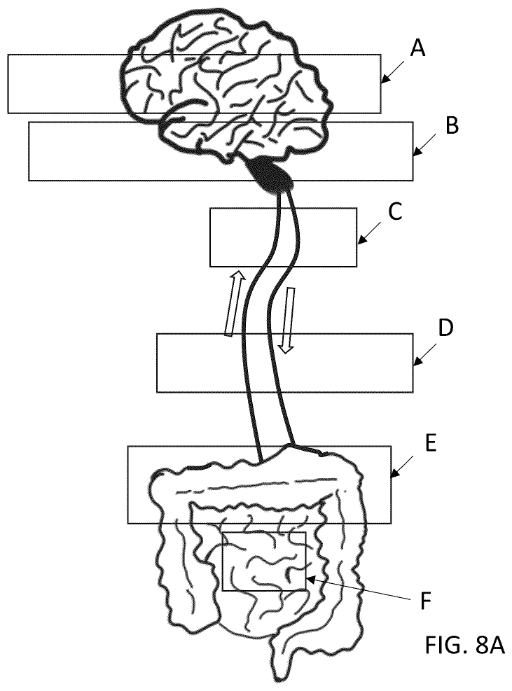

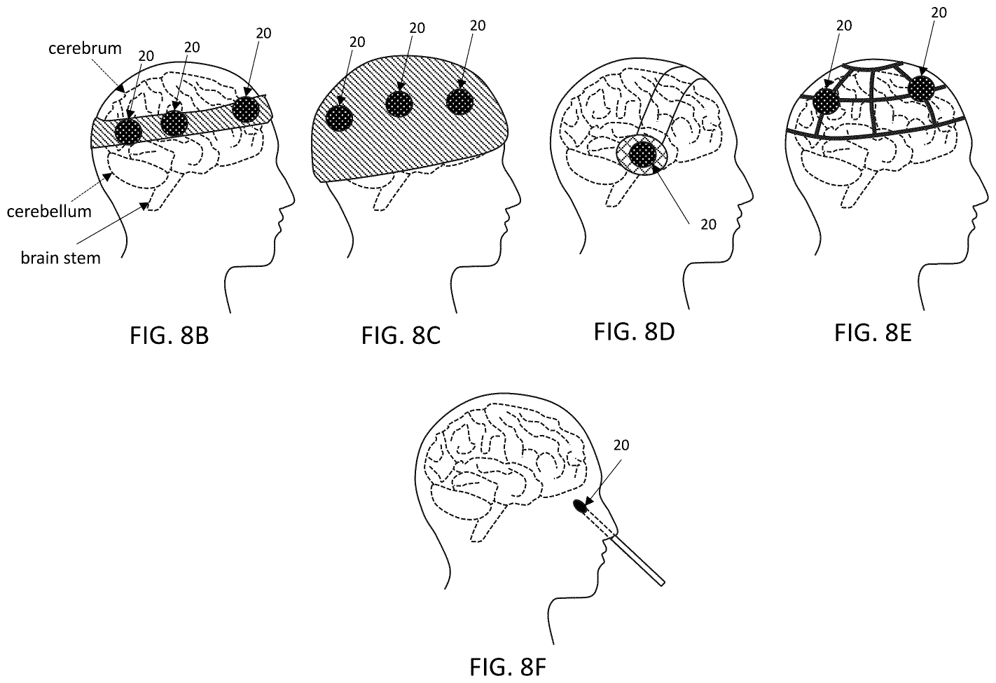

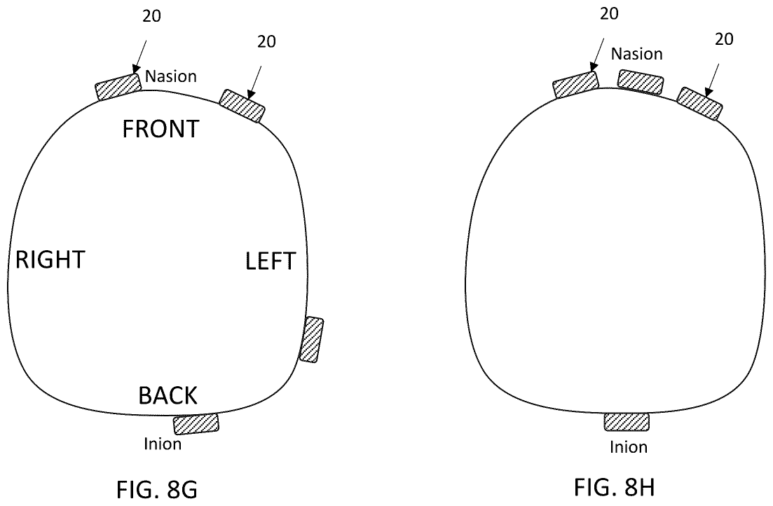

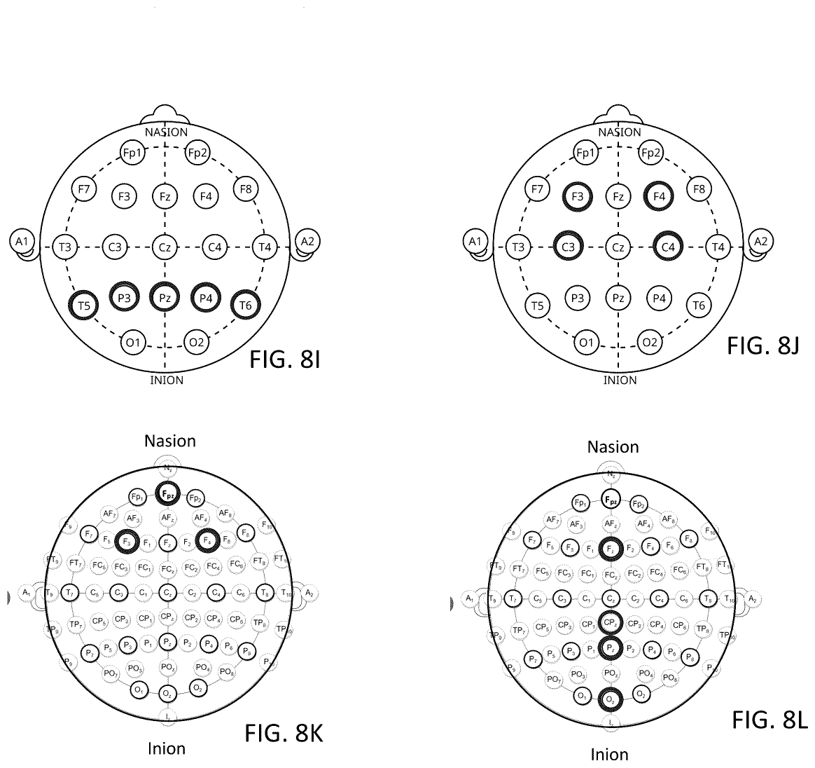

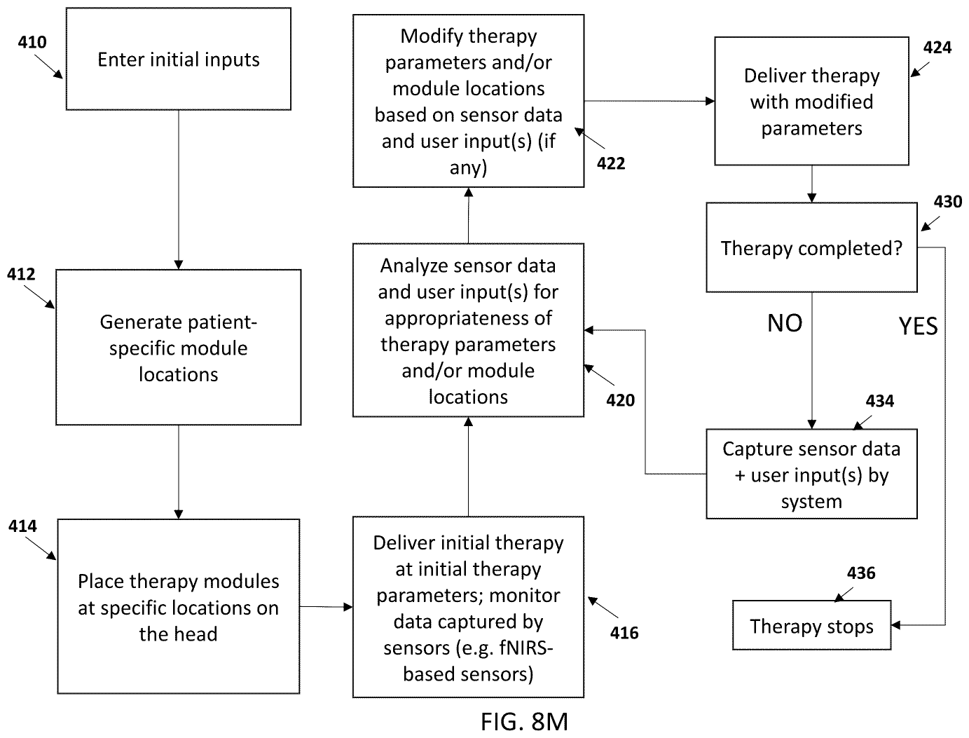

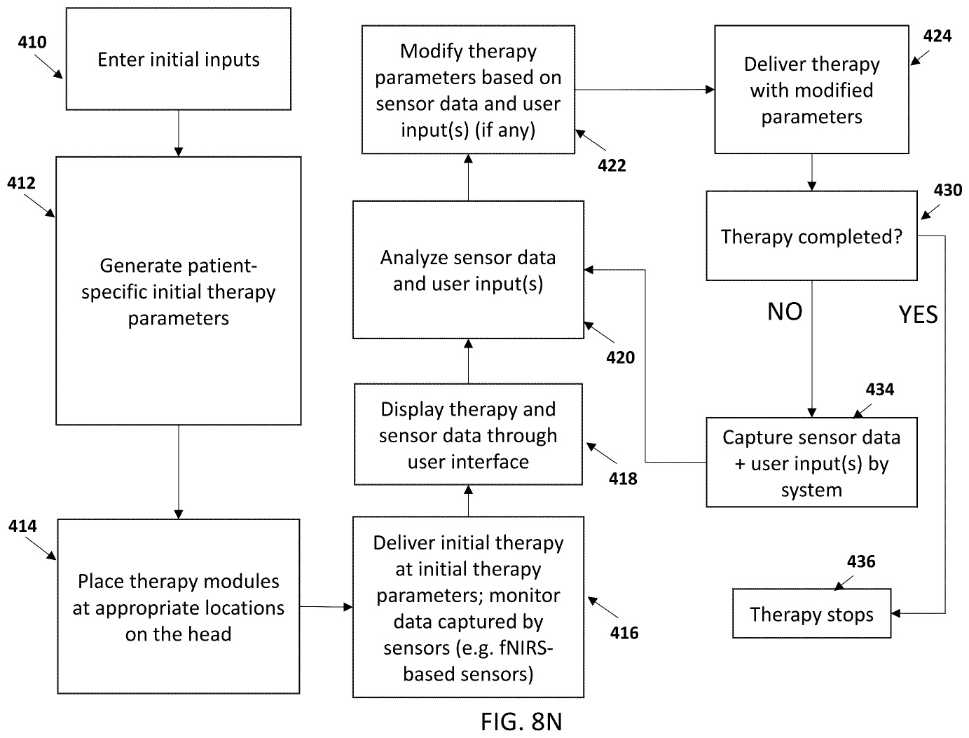

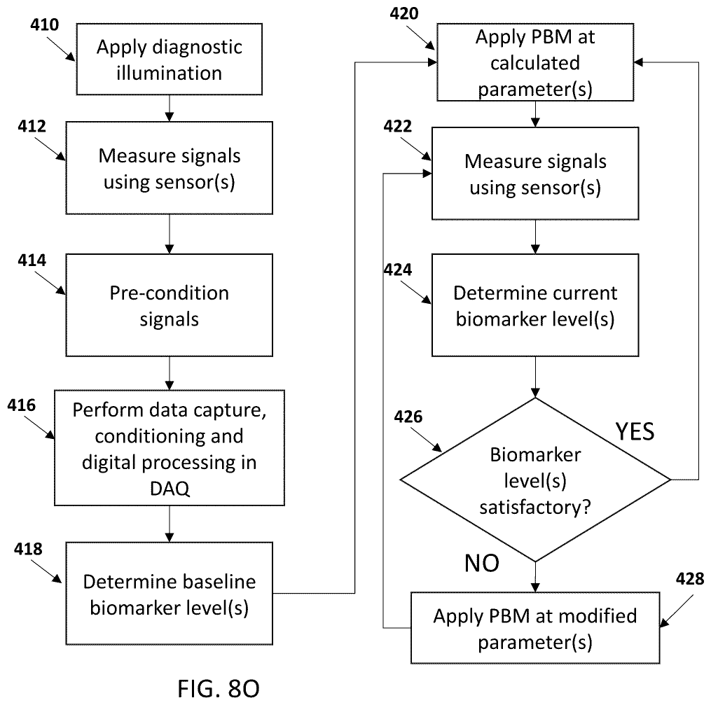

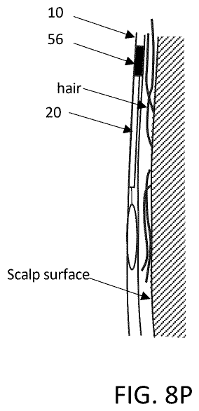

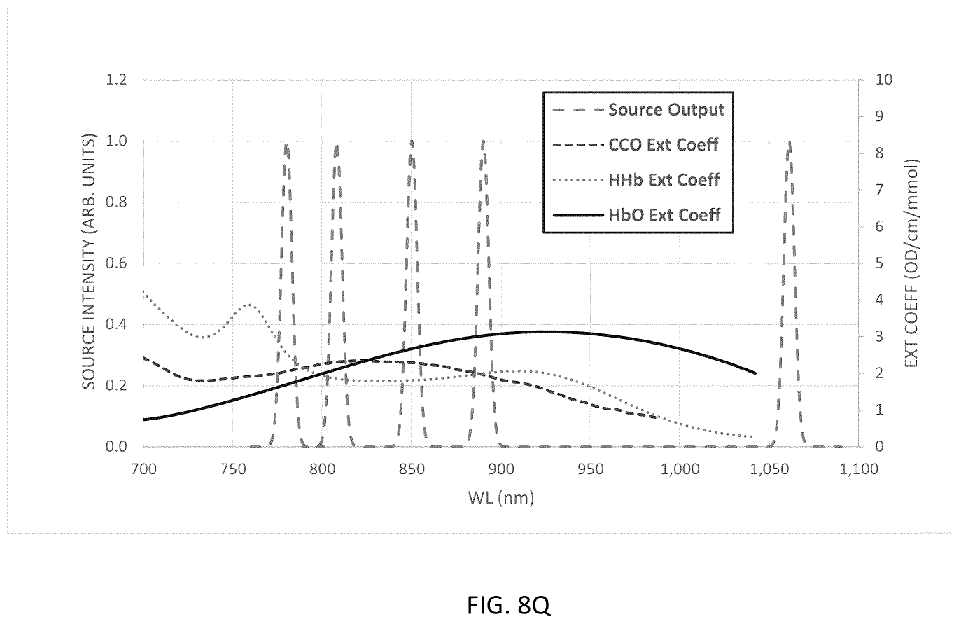

In a non-limiting example, a first wearable device 10 (examples shown in FIGS. 1C-1D, etc.) may be placed to deliver PBM to a region of the intestine and a second wearable device 10 (examples shown in FIGS. 8B-8L, etc.) may be placed on the head to deliver PBM to a region of the brain. At least one module 20 may be programmed to deliver customized PBM to the intestine and may be attached to the first wearable device 10. At least one module 20 may be programmed to deliver customized PBM to the brain and may be attached to the second wearable device 10, In some embodiments, the first and the second modules may have the same hardware configuration, but may be programmed differently to deliver PBM that differs in at least one parameter. Any of the programming steps disclosed herein may be performed using a software application (e.g., app 79) loaded on a mobile device or other electronic device. Any of the programming steps disclosed herein may be performed using a wireless connection or a wired connection.

One or more working parameters may be adjusted before, during, or after the delivery of PBM. Such adjustment(s) may be performed by one or more of: manually by the user through a user interface, automatically by one or more modules 20, automatically by an application loaded on an electronic device in communication with one or more modules 20 and/or sensors 56, using data from one or more sensors 56, etc. Examples of such adjustments are described in FIG. 3A-3B, 6A-6B, 7A-7C, 8M-8O, etc.

At step 780, one or more therapies are delivered to the patient. At step 782, the therapy is checked for satisfactory performance. This may occur a few days after the patient starts the therapy. Satisfactory performance may be determined through one or more of: adverse effects, improvement in symptoms, reduction of pain or other discomfort, one or more medical outcomes, etc. If the performance is deemed to be satisfactory, the one or more therapies are continued to be delivered to the patient. If the performance is deemed to be unsatisfactory, the method returns to step 770 where one or more treatment parameters are adjusted. In one such example, the energy dose per session may be increased or decreased based on the performance of the therapies. In another example, the location and/or number and/or type of one or more modules 20 may be changed based on the performance of the therapies.

FIG. 3B shows a flow chart of an example method of delivering adjustable PBM and other therapies to a patient. At step 783, a therapy is selected for the patient. Examples of therapies are disclosed in this disclosure. At step 784, one or more patient parameters are determined. Examples of patient parameters include, but are not limited to: location(s) of one or more organs, location(s) of one or more diseases or disorders, skin color and other parameters, location and thickness of tissues such as adipose tissue, location of one or more target tissues, location of symptoms (e.g., pain, ache, discomfort, inflammation, etc.) experienced by the patient, one or more medical conditions of the patient, etc. At step 785, one or more treatment parameters are determined. These therapy parameters are determined based on one or more patient parameters obtained at step 784. At step 786, one or more therapy modules 20 are selected. One or more therapy module(s) 20 may be selected based on one or more considerations; examples of which include, but are not limited to: location(s) of the therapies, type of therapy to be delivered, therapy parameters, etc. At step 787, desired treatment parameters are set or adjusted. This may be done by programming or otherwise setting such treatment parameters on one or more modules 20 or devices controlling one or more modules 20. At step 788, the location(s) of one or more modules 20 relative to the patient is determined. In some embodiments, the location(s) of one or more modules 20 is determined by the location(s) of one or more target organs. In some embodiments, the location(s) of one or more modules 20 is determined by the location(s) of pain or other feeling(s) experienced by the patient. At step 789, one or more modules are connected to a wearable device 10. This may be done by selecting the appropriate location of the modules 20 on wearable device 10 and connecting the modules 20 to wearable device 10. At step 790, one or more therapies are delivered to the patient. At step 791, the therapy location(s) are checked. It is determined if the therapy locations are satisfactory. This may occur a few days after the patient starts the therapy. Satisfactory location may be determined through one or more of: adverse effects, improvement in symptoms, reduction of pain or other discomfort, one or more medical outcomes, etc. If the therapy location is deemed to be satisfactory, the method proceeds to step 792. If the therapy location is deemed to be unsatisfactory, the method returns to step 788 where the adjusted location(s) of one or more modules 20 relative to the patient is determined. At step 792, the therapy parameters are checked for satisfactory performance. This may occur a few days after the patient starts the therapy. If the therapy parameters are deemed to be satisfactory, the one or more therapies are continued to be delivered to the patient at step 790. If the therapy parameters are deemed to be unsatisfactory, the method returns to step 787 where one or more treatment parameters are adjusted and/or set. In one such example, the energy dose per session may be increased or decreased based on the performance of the therapies. In another example, the location and/or number and/or type of one or more modules 20 may be changed based on the performance of the therapies.

Various methods and devices disclosed herein may be used to diagnose and/or treat any back pain condition, its symptoms, or other musculoskeletal disorders. Back pain is defined as pain or discomfort felt in any region around the spine extending from the level of the first cervical vertebra until the coccyx. Examples of back pain conditions that may be treated by the devices described herein include, but are not limited to: spondylolisthesis, chronic lower back pain, spinal stenosis, muscle strain or sprain, arthritis-related back pain, fibromyalgia, sciatica, post-surgical back pain, discogenic back pain, back pain of unknown origin, etc.

Wearable devices 10 for treating back pain conditions may be designed to be worn circumferentially around the torso, waist, or other suitable bodily regions. Such wearable devices 10 may include one or more attachment means in the form of wraps or belts (e.g., a belt that is worn around the lower back or waist) that are worn circumferentially around the one or more bodily regions. Such wearable devices 10 may include one or more attachment means (e.g., patches worn around the lower back or waist) that do not extend around the complete circumference of the body or partially extend around a circumference of the body. As discussed elsewhere in this specification, wearable devices 10 may include one or more light sources 36, sensors 56, etc. Data collected from one or more sensors 56 may be analyzed to identify disease or treatment patterns, identify disease sites, identify sites of origin of pain or discomfort, refine treatment protocols, provide feedback to healthcare providers, etc. One advantage of the devices described herein is the ability to deliver therapy across the entire circumference of the user's body. This allows one or more therapies to be delivered from multiple regions over the body. For example, one or more therapies may be delivered simultaneously from the front of the user's body as well as from the back of the user's body. This is useful in treating conditions such as back pain, pelvic pain, endometriosis-related pain, etc.

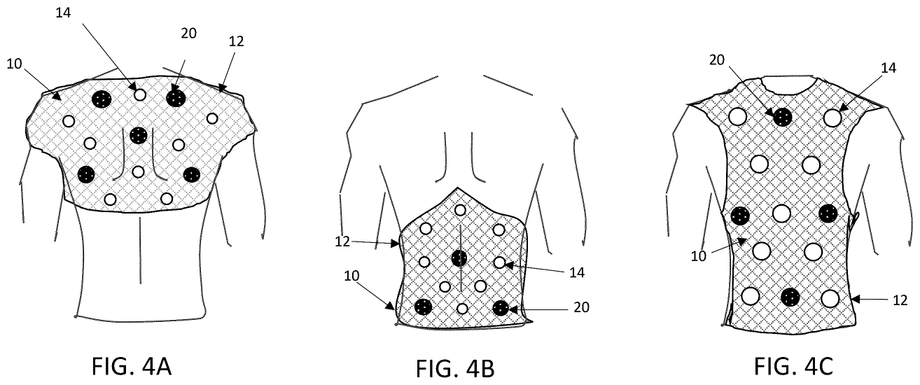

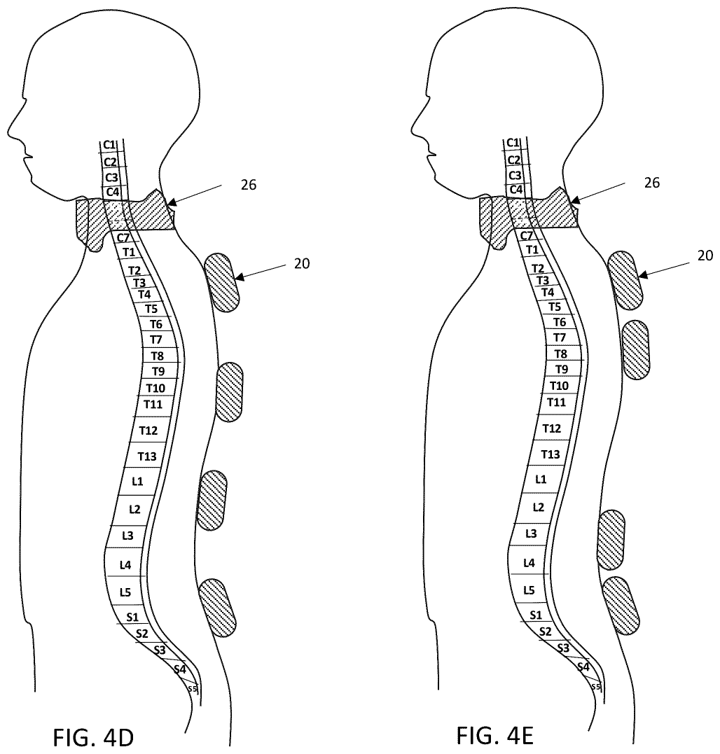

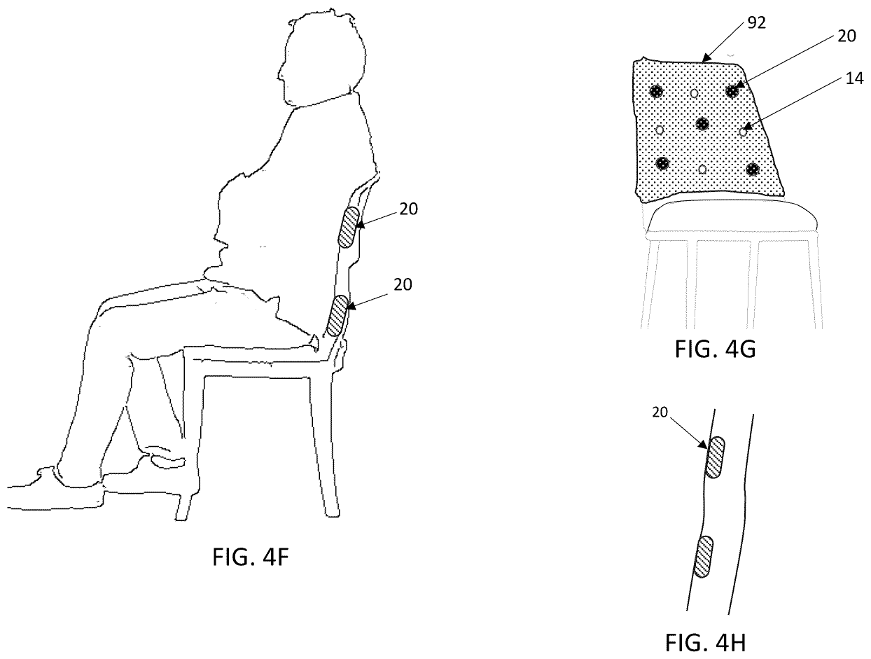

FIGS. 4A-4C show various embodiments of devices to treat back pain. Such embodiments may include one to 40 modules, the locations of which may be modified as desired. One or more anatomical and/or pathological markers may be used to guide the position of one or more modules 20 or one or more wearable devices 10 disclosed herein. Examples of such anatomical and pathological markers include, but are not limited to: locations of specific vertebrae, location of muscles, non-spinal bone regions, location(s) of pain felt by the patient, response to a pain-generating stimulus, response to a pain reducing stimulus, response to a pain blocking action, locations of nerves, and locations of abnormalities seen on imaging (e.g., X-ray studies, CT scans, MRI scans, etc.), and their combinations.

FIG. 4A shows an example wearable device 10 positioned on an upper back region such that the wearable device extends up to one or both shoulders of a patient. FIG. 4B shows an example wearable device 10 positioned on a lower back region such that the wearable device extends down to a waist of a patient. FIG. 4C shows an example wearable device 10 positioned on a back of the patient such that the wearable device extends from one or both shoulders of a patient to a waist of the patient. The embodiments shown in FIGS. 4A-4C may be designed in the form of wraps, jackets, half-jackets, etc. that position one or more modules 20 around the back of the patient. Such embodiments may include multiple module attachment sites 14 where the user can place one or more modules 20. The position of such wearable devices 10 may be adjusted relative to the position of one or more spine regions.

The position of modules 20 on such wearable devices 10 may be adjusted relative to the position of one or more spine regions. Such wearable devices 10 embodiments are suited for home or office use where they can be worn under regular clothing. Such wearable devices 10 embodiments may be used to deliver light or other therapies to a portion of, an entirety of, or a majority of back region.

Light based therapies disclosed herein may be combined with other suitable therapies to provide combination therapy to the user. FIGS. 4D and 4E shows a side view of a user using a combination therapy including a light-based therapy and an electrical stimulation-based therapy. Several examples of light-based modules 20 are disclosed in this specification. Examples of electrical stimulation-based therapy are shown in FIGS. 2A and 2B. Such combination therapy may include two types of therapies that are delivered to two different bodily regions to achieve two separate effects. In one such example shown in FIG. 4D, electrical stimulation-based therapy is delivered to an area near the neck of the user to cause mental or muscle relaxation. One or more modules 20 positioned on the back of the patient may be used to deliver light-based therapy to ease pain or discomfort around the spine. As shown in FIG. 4E, the location of one or more such therapies may be changed to deliver one or more therapies at optimal target location(s). Examples of therapies that may be combined with light-based therapies disclosed herein to create combination therapies include, but are not limited to: vibration-based therapy, thermal therapy (e.g., hot or cold therapy), topical medications or other medications, sensory therapy, etc. Such therapies may be delivered by one or more elements present on device 10 such as vibrating elements, thermoelectric cooling elements, etc.

FIGS. 4F-4H show embodiments of furniture or other household items that contain one or more modules. FIG. 4F shows an example of a chair adapted for delivering PBM to the back of a user through one or more modules 20 that are mechanically connected to or are a part of the chair. FIG. 4G shows an example of a device for attaching one or more modules 20 to objects commonly found in the household or in the work or commercial environments. Examples of such objects include, but are not limited to: chairs or other objects designed for sitting or relaxing (e.g. chairs, lounge chairs, sofas, beach chairs, pool chairs, beds, etc.), spas, hot tubs, jacuzzies, massage chairs or other objects that provide massages or vibrations, bath tubs, etc. One or more modules 20 may be permanently or reversibly attached at one or more module attachment sites 14 located on one or more magnetic attachments 90 or mechanical attachments 92. These attachments may vary in design and functionality based on the object type, target regions on the user's body, etc. These attachments may be adjustable (e.g., adjustable straps, attachments of varying size and shape, adjustable or moveable modules 20, etc.) based on the object type, target regions on the user's body, etc. Some examples of such mechanical attachments 92 include, but are not limited to: bands or straps that fully or partially envelop the objects, covers or caps that go over one or more portions of the objects, mechanical attachments that adhere to one or more portions of the objects (e.g., using magnetic force, using Velcro™ mechanisms, using adhesives, using vacuum, etc.), FIG. 4H shows an example of one or more modules 20 temporarily embedded in or permanently attached to objects commonly found in the household or in the work or commercial environments. As disclosed elsewhere herein, the placement of one or more modules 20 and therapy delivered through such modules may be used for one or more of: diagnosing the source(s) of pain, diagnosing the location(s) of pain, etc.

Some specific applications of treating musculoskeletal conditions are given below. Methods and devices disclosed herein may be used to provide relief from chronic back pain, especially chronic low back pain by reducing inflammation, stimulating tissue repair, providing analgesic effects, etc. Since low back pain often originates in the lumbar vertebrae or surrounding tissues, the therapy delivery devices disclosed herein may be placed around L1 to L5 vertebrae. The spinous processes and other features of the vertebrae, vertebral furrow, locations of one or more nerves, locations of one or more muscles, locations of one or more ligaments, or other anatomical markers may be used to guide the placement of modules 20 and other device regions. Multiple modules 20 may be placed if pain or other discomfort originates from multiple sources. In some embodiments, a therapy is delivered to a general area in addition to specific source(s) of pain or discomfort. Often, a specific cause of back pain cannot be found. In such cases, the user may self-adjust the location(s) of one or more modules 20 and other therapy devices to obtain relief from back pain.

Methods and devices disclosed herein may be used to provide relief from sciatica. This may be achieved by one or more of: reducing nerve irritation (especially sciatic nerve inflammation), reducing inflammation, alleviating pain radiating from the lower back into the legs, etc.

Methods and devices disclosed herein may be used to provide relief from muscle strain and spasm. This may be achieved by one or more of: easing muscle tension and easing spasms, especially after injury or overuse.

Methods and devices disclosed herein may be used to manage some of the symptoms associated with degenerative disc disease by promoting tissue repair and reducing inflammation around the affected discs.

Methods and devices disclosed herein may be used to manage some of the symptoms associated with conditions such as myofascial pain, spondylosis, and spondylolisthesis. This may be achieved by using one or more modules 20 at suitable locations for achieving one or more of: reducing nerve irritation (especially of pinched or inflamed nerves), reducing inflammation, etc.

Methods and devices disclosed herein may be used to manage arthritis-related pain, especially back pain. This may be achieved by using one or more modules 20 for one or more of: reducing inflammation, blocking pain transmission, etc.

Methods and devices disclosed herein may be used to manage some of the symptoms associated with spinal stenosis. Stenosis of the spinal canal compresses the spinal cord and one or more nerves. Managing symptoms may be achieved by using one or more modules 20 for one or more of: reducing nerve irritation, reducing inflammation, etc.

Methods and devices disclosed herein may be used to enhance post-surgical recovery. One or more modules 20 may be placed at location(s) around the surgical site to achieve one or more of: reducing inflammation, reducing pain, increasing blood flow, relaxing muscles, etc.

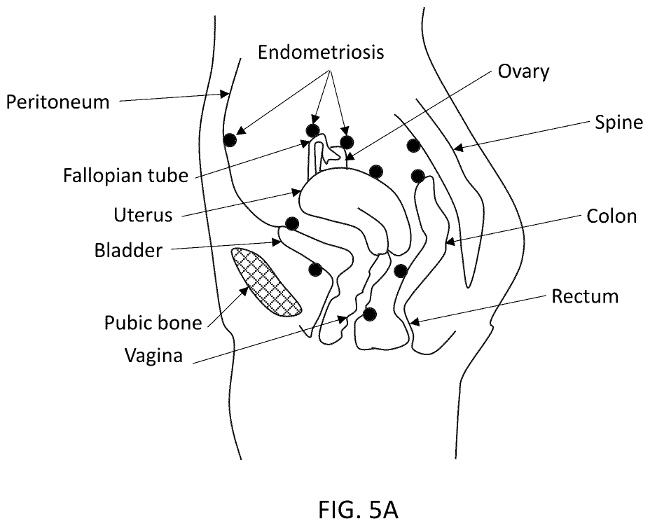

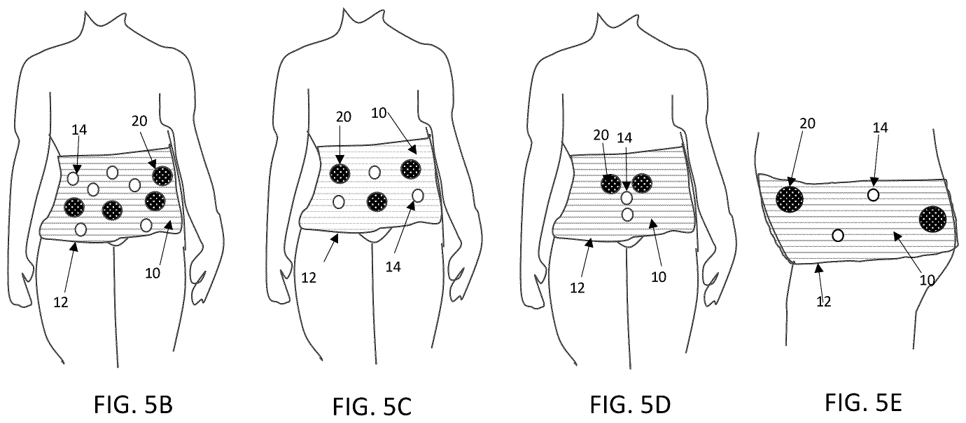

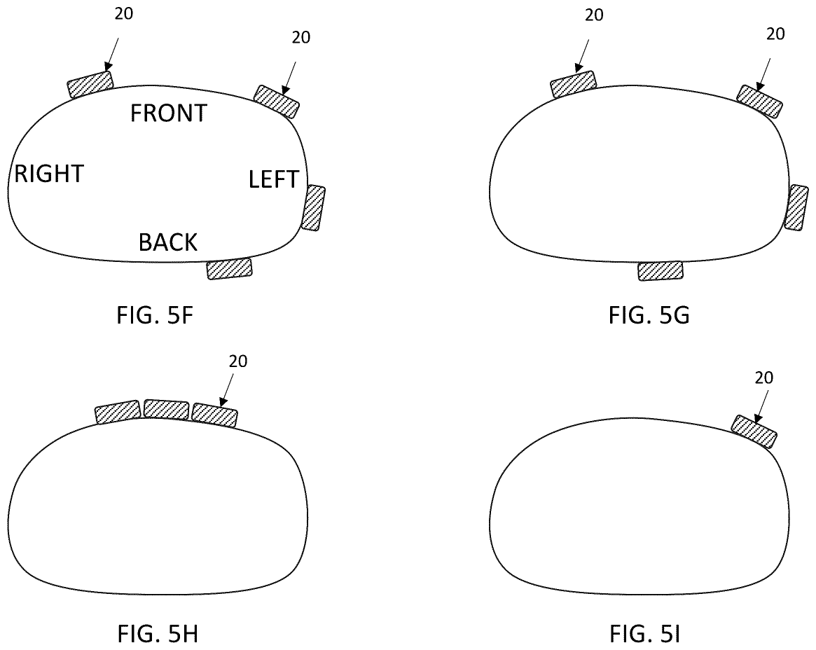

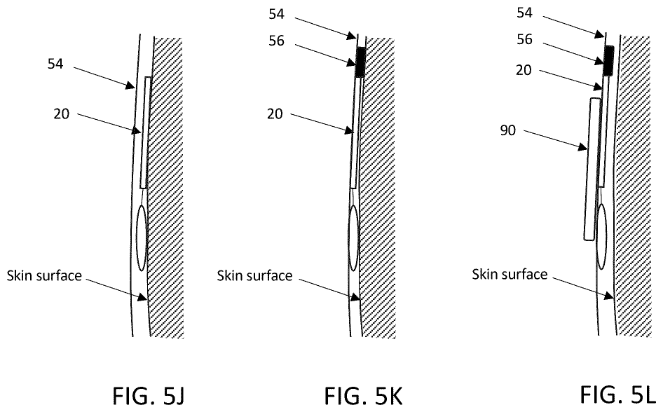

Any of the methods and devices disclosed herein for diagnosing and/or treating back pain conditions, their symptoms, or other musculoskeletal disorders may incorporate features described elsewhere herein. For example, such methods and devices may incorporate closed-loop feedback mechanisms; examples of which are shown in FIGS. 6A and 6B. Such devices may be designed to be work discretely under the clothing or be a part of clothing. Examples of such embodiments are shown in FIGS. 5J and 5K. Such methods and devices may incorporate AI or ML based models; examples of which are shown in FIGS. 7A-7C. Such methods and devices may be used to deliver adjustable PBM and other therapies to a patient as shown in FIGS. 3A and 3B.

One or more methods disclosed herein may be used for treating one or more symptoms (e.g., pain, inflammation, sensitivity, infertility, etc.) of female-specific conditions. Examples of such female-specific conditions include, but are not limited to: endometriosis, adenomyosis, pelvic pain, pelvic congestion syndrome, dysmenorrhea, dyspareunia, infertility, etc. One or more methods disclosed herein may be used to treat one or more symptoms (e.g., pain, inflammation, sensitivity, infertility, etc.) of pelvic conditions. Several embodiments of such methods as given below. Although endometriosis is used as an example of a clinical condition in methods disclosed in FIGS. 5A-5K, such methods may be used for treating symptoms of one or more pelvic conditions or female-specific conditions. For example, conditions such as endometriosis and their symptoms are difficult to treat using targeted therapies. Embodiments disclosed herein may be used to provide targeted therapies for easing pain and other symptoms of conditions such as endometriosis.

FIG. 5A shows a view of pelvic anatomy with example locations of endometriosis that can be targeted using one or more method and/or device embodiments described herein for symptom relief. As shown in FIG. 5A, endometriosis lesions that are treated using one or more methods and devices disclosed herein may be present in regions including, but not limited to: peritoneal layers, fallopian tubes, uterus, ovary, colon, small intestine, rectum, vagina, bladder, etc. The placement of device components (e.g., one or more modules 20) may be determined based on the location of one or more endometriosis lesions, sites of pain, sites of nerves, etc. Any suitable placement location(s) of one or more modules 20 disclosed herein may be used to target the symptoms of adenomyosis at any location(s) shown in FIG. 5A. One advantage of the modular nature of the methods and devices disclosed herein is that light therapy may be delivered from multiple locations and orientations over one or more the anatomical regions.

Diagnosis and/or characterization of endometriosis or other conditions disclosed herein may be performed by techniques including, but not limited to:
1. Laparoscopy.
2. Histopathology analysis.
3. Magnetic Resonance Imaging (MRI): MRI may especially be used for identifying deep endometriosis, deep infiltrating endometriosis, etc.
4. Artificial Intelligence (AI): AI-based tools may be used to analyze clinical data disclosed herein including, but not limited to: clinical symptoms, Transvaginal Ultrasound (TVUS) data, MRI data, etc. AI-based tool may be used to improve the detection accuracy of imaging results.
5. Tissue biopsy.
6. TVUS: TVUS may especially be used for detecting ovarian endometriomas and deep endometriosis.
7. Endoscopic rectal ultrasound: This technique may especially be used for detecting bowel endometriosis.
8. Transvaginal Elastosonography: In this technique, tissue stiffness may be measured using ultrasound to identify endometriosis, especially deep endometriosis.
9. Rectal Water Contrast-Transvaginal Ultrasound: This technique may especially be used for diagnosing endometriosis in the deep posterior regions of the pelvis.
10. 3D Ultrasonography: This technique may especially be used for diagnosing bladder endometriosis.
11. Positron emission tomography (PET): PET-based methods such as PET-CT scans that combine a positron emission tomography (PET) scan and a computed tomography (CT) scan to create detailed 3D images of the body may be used to diagnose endometriosis.
12. Magnetic resonance elastography (MRE).
13. Symptom analysis: Examples of such methods include clinical methods that analyze symptom information such as location of symptoms, type of symptoms, duration of symptoms, correlation of symptoms with other parameters, etc.
14. Blood flow data
15. Microvasculature analysis.

One or more of the above may be combined. For example, information obtained from laparoscopy may be combined with histopathology information and used for diagnosis, disease localization, treatment planning, etc. In another example, an AI-based method that analyzes MRE and other imaging data may be used for diagnosis, disease localization, treatment planning, etc. One or more of: locations of endometriosis and other pelvic pain-generating conditions, locations of placement of one or more modules 20, and one or more therapy parameters, etc. may be determined by one or more of the above-mentioned techniques.

In some embodiments, MRI imaging is performed on a patient. An AI-based model may be used to analyze data from the MRI imaging study. The AI-based model may be used to analyze one or more pelvic organs to determine one or more digital biomarkers. Examples of such biomarkers include, but are not limited to: tissue properties, location(s) of endometriomas, etc. This analysis may be used to determine the locations of one or more endometriosis lesions. Such information is then used to performed one or more of the methods disclosed herein to deliver a personalized therapy to the patient. In one such embodiment, information obtained from any of the above-mentioned techniques may be used for one or more of:
1. Determining the number of modules 20 to be used to treat the patient;
2. Selecting the type of wearable device 10 to be used;
3. Adjusting wearable device 10 relative to the patient's anatomy;
4. Adjusting position(s) of one or more modules 20 relative to wearable device 10;
5. Determining one or more treatment parameters; and 6.

Delivering therapy to the patient through one or more modules 20. In one such embodiment, information obtained from any of the above-mentioned techniques may be used for one or more of:
1. Determining the locations of one or more regions of the uterus;
2. Determining the number of modules 20 to be used to treat the patient's uterus;
3. Selecting the type of wearable device 10 to be used;
4. Adjusting wearable device 10 relative to the patient's anatomy (especially relative to the patient's uterus);
5. Adjusting position(s) of one or more modules 20 relative to wearable device 10;
6. Determining one or more treatment parameters; and 6. Delivering therapy to the patient's uterus through one or more modules 20.

FIG. 5B shows a front view of a user wearing an example wearable device for treating a pelvic condition such as endometriosis. In this example, the user is using four modules 20 for the treatment. These four modules 20 are attached to four of 10 module attachment sites 14. The strap or wearable device 12 is connected circumferentially around the torso of the patient. As discussed elsewhere, the number, type, location, treatment parameters, etc. of one or more modules 20 may be changed based on a desired effect.

FIG. 5C shows a front view of a user wearing another example wearable device for treating a pelvic condition such as endometriosis. In this example, the user is using three modules 20 for the treatment. These three modules 20 are attached to three of six module attachment sites 14. The strap or wearable device 12 is connected circumferentially around the torso of the patient. As discussed elsewhere, the number, type, location, treatment parameters, etc. of one or more modules 20 may be changed based on a desired effect.

FIG. 5D shows a front view of a user wearing an example wearable device for treating a uterine or peri-uterine condition. In this example, the user is using two modules 20 for the treatment. These two modules 20 are attached to two of four module attachment sites 14. The strap or wearable device 12 is connected circumferentially around the torso of the patient. These two modules 20 are placed to deliver therapy to one or more uterine or peri-uterine regions. As discussed elsewhere, the number, type, location, treatment parameters, etc. of one or more modules 20 may be changed based on a desired effect.

FIG. 5E shows a side view of a user wearing an example wearable device for treating a pelvic condition such as endometriosis. In this view of the embodiment, two modules 20 are seen. The strap or wearable device 12 is connected circumferentially around the torso of the patient. This figure illustrates the advantage of the present described implementations in terms of the ability to place modules 20 circumferentially around the torso, even on the back or the sides of the patient and deliver one or more therapies to the patient from multiple locations, including from the back or the sides of the patient. As discussed elsewhere, the number, type, location, treatment parameters, etc. of one or more modules 20 may be changed based on a desired effect.

FIGS. 5F-5I show a cross-section through a patient's torso showing various embodiments of the circumferential placement of one or more modules 20 disclosed herein. The nomenclature used to define the regions of the patient's torso are shown in FIG. 5F. FIG. 5F shows an embodiment of a wearable device having four modules 20 placed circumferentially around the patient's torso. Two modules 20 are placed on the front of the patient, one module 20 is placed on the left side of the patient, and one module 20 is placed on the back of the patient. The location of one or more modules 20 in the embodiments disclosed herein may be modified. For example, the patient may modify the placement shown in FIG. 5F by adjusting the position of module 20 placed on the back of the patient as shown in FIG. 5G. FIG. 5H shows an embodiment of a wearable device having three modules 20 placed around the patient's uterus or peri-uterine regions. FIG. 5I shows an embodiment of a wearable device having a single module 20 placed to target a specific abdominal region or organ.

In some embodiments, an imaging study is performed on a patient. The imaging data is analyzed (e.g., using AI-based methods, by a radiologist, etc.) to diagnose the presence, location, severity, etc. of a pelvic or other bodily condition, examples of which are given elsewhere in this specification. Based on the analysis of the imaging data, the patient can self-administer one or more therapies disclosed herein. For example, the patient can decide the location(s), type(s), parameter(s), etc. of one or more therapies disclosed herein.

Any of the methods and devices disclosed herein may be used to diagnose a condition based on a clinical or physiological effect. For example, location of one or more back or pelvic pain sites may be determined by placing one or more modules 20 at specific sites and determining a clinical or physiological effect felt by the patient. The patient may reposition one or more modules 20 and/or change one or more treatment parameters until the patient experiences a clinical or physiological effect. The location of one or more modules 20 and one or more treatment parameters may be used to determine one or more characteristics of one or more conditions. Such characterization includes determining parameters including, but not limited to: presence/absence of a condition, location(s) of one or more lesions, location(s) of one or more inflammation points, location(s) of one or more bodily regions generating and/or transmitting pain, etc. In one example, a pelvic condition such as endometriosis is characterized by such methods. In one example, back pain is characterized by such methods. Thus, diagnosis and/or characterization of one or more conditions (e.g., pelvic conditions) may be performed based on the symptom relief or other changes felt by the patient. Placement of one or more sensors 56, type(s) of one or more sensors 56, one or more treatment parameters, etc. may be modified by the patient in this process.

Such methods are especially useful for conditions such as endometriosis or back pain that are difficult to diagnose and/or characterize. In one such embodiment, the patient delivers a therapy to one or more bodily regions. The pain or other symptom relief or other changes felt by the patient are then used to determine the location of one or more regions of endometriosis, one or more pain/inflammation sites, etc. If such a patient undergoes an imaging study (e.g., MRI scans. CT scans, ultrasound exams, etc.), the imaging data could be interpreted after accounting for the pain or other symptom relief or other changes felt by the patient.

Methods and devices disclosed herein may also be used to diagnose dysmenorrhea or other conditions where the uterus is a source of pain or discomfort. In one such embodiment, one or more modules 20 are used to deliver a therapy to one or more uterine or peri-uterine regions (e.g. abdominal muscles, fascia, etc.). The pain or other symptom relief or other changes felt by the patient are then used to determine the location of one or more regions of dysmenorrhea (e.g., primary dysmenorrhea, secondary dysmenorrhea, etc.), one or more pain/inflammation sites, etc. If such a patient undergoes an imaging study (e.g., MRI scans. CT scans, ultrasound exams, etc.), the imaging data could be interpreted after accounting for the pain or other symptom relief or other changes felt by the patient.

Methods and devices disclosed herein may also be used to treat one or more pelvic pain conditions including, but not limited to: dysmenorrhea, chronic pelvic pain, endometriosis-related pain, pain of uterine origin, etc. by targeting one or both of: a pelvic organ, an abdominal muscle, and a back muscle. Abdominal muscles include, but are not limited to: pyramidalis, rectus abdominus, external obliques, internal obliques and transversus abdominis. In some embodiments, energy is delivered to one or more abdominal skeletal muscles to treat a pelvic pain condition. Such energy delivery may be used to modify abdominal muscle activity. For example, dysmenorrhea may be treated by delivering energy to the uterus and one or more abdominal muscles.

FIG. 5J shows an example wearable device module configured to be worn underneath clothes. Such modules 20 may be designed such that they are not visible under clothing 154. Thus, wearable 10 embodiments may be designed to be worn throughout the day including at work or while traveling. The light emitted by such modules 20 may be masked or shielded such that the light is not visible under clothing 154. In some embodiments, a module 20 includes one or more sensor 56 and one or more light sources to provide light therapy to an area of interest. The thickness of module 20 embodiments may be reduced using design features and techniques, examples of which include but are not limited to: miniaturization of components, use of flexible and slim batteries, material selection, integrating one or more components with clothing, and modular designs. Details of such design features and techniques are described below.

Miniaturization of Components may be achieved by one or more of:
1. Advanced Circuit Integration: The internal components of module 20 (e.g., sensors, batteries, and processing units) may be miniaturized using state-of-the-art technologies such as micro-electromechanical systems (MEMS) and system-on-chip (SoC) technologies to allow multiple functionalities to be incorporated into a smaller footprint.
2. Flexible Printed Circuit Boards (FPCB): Replacing rigid PCBs with flexible circuit boards may be used to reduce the overall thickness of module 20 by allowing it to conform to the body's contours, thus creating a sleeker, flatter design.

Flexible and Slim Batteries may be designed using technologies such as:
1. Thin-film Lithium Batteries: Thin-film battery technologies can be used to power module 20. These batteries have a much thinner profile than traditional coin or cylindrical batteries, which will create a sleeker, flatter design.
2. Energy Harvesting Mechanisms: Implementing energy harvesting from body heat, motion, or ambient light could reduce or eliminate large batteries, as the device can be powered by ambient energy sources.

Suitable materials may be selected to achieve or create:
1. Flexible Enclosures: Flexible, biocompatible materials such as silicone or polyurethane may be used to create a flexible module 20 that flexes and conforms to skin surface, reducing the thickness of module 20.
2. Heat Dissipation: Since thickness is often a function of heat management, incorporating efficient heat-conducting materials or passive cooling methods (e.g., graphite layers) may be used to create slimmer designs while maintaining thermal safety.

One or more module 20 components may be integrated directly into garments such as underwear, outerwear, or clothing patches. This would make the user feel more comfortable while wearing wearables 10 including one more modules 20.

Modular designs may be achieved using a distributed component layout. Thicker components may be converted to smaller sub-components and arranged over a larger area to reduce the thickness of modules 20. These sub-components may communicate wirelessly or using flexible wires (e.g., using ultra-thin interconnects) with each other.

Even though several embodiments disclosed herein relate to wearable devices including detachable modules 20, one or more modules 20 may be permanently attached to or embedded within a wearable device 10. In such embodiments, the location(s) of module(s) 20 relative to the patient may be adjusted by adjusting the location of wearable device 10 relative to the patient. FIG. 5K shows an embodiment of a wearable device with one or more sensors embedded into clothing. Such clothing 54 may be worn such that it directly contacts the skin surface of a patient. Such clothing 54, modules 20, and their components may be built using flexible substrates that allow them to conform to the skin's surface so that the patient can wear it long-term without discomfort.

In some embodiments, clothing 54 (wearable device 10) includes a sensor 56, a flexible substrate, and a skin interface. Any of the sensors 56 disclosed herein may sense and/or measure parameters including, but not limited to: blood flow, nitric oxide levels, heart rate, body temperature, skin hydration, skin movement, bodily movement, microvasculature data, etc. The sensor 56, embedded into clothing 54, is positioned close to the skin surface or in contact with the skin surface. This allows sensor 56 to collect data from the patient. Sensor 56 may measure biometric data from the patient; examples of which are disclosed elsewhere herein. These data may be used to guide the application of light therapy to one or more areas of interest. The structure holding sensor 56 and other components may be made from a thin, flexible material. This structure may be lightweight and able to bend to conform with the natural flexing and movement of the skin. This ensures that the sensor remains securely in place even during movement, without causing irritation or detaching from the skin. The bottom layer of the module 20 or components thereof may adhere to the skin, ensuring continuous contact with the skin surface. Various portions of wearable device 10 may be designed with hypoallergenic materials. Various portions of wearable device 10 may be designed such that they are “breathable” i.e. allowing air and moisture, especially moisture from sweat to pass through. This creates a more comfortable experience for the patient by regulating temperature and moisture levels.

One or more components of modules 20 or other components of device 10 may be waterproof such that the clothing 54 (wearable device 10) may be washable. The comfort experienced by a user when wearing clothing 54 (wearable device 10) may be increased by one or more of:
selecting fabrics with improved qualities such as breathability, softness, hypoallergenic nature, and moisture absorption. Such fabrics may include natural fibers (examples include, but are not limited to: cotton, linen, silk, and bamboo), blended materials combining natural and synthetic fibers (e.g., cotton-polyester blends), or stretch fabrics such as Spandex or Elastane
using knitted fabrics which are generally elastic and softer than woven fabrics
using fabrics with breathable weaves such as looser weaves or mesh fabrics to improve airflow and temperature regulation
using fabrics treated with finishes and/or treatments such as softening agents to improve their feel and antimicrobial treatments to prevent odors and bacterial growth
seam placements that reduce chafing and irritation
tag less designs
adjustable features (examples of which include, but are not limited to: drawstrings, Velcro®-type attachments, elastic bands, and adjustable straps) to allow users to customize the fitting
soft linings made of fabrics like fleece or satin to reduce friction with skin
breathable inner linings to create a “dry feel” on the skin

FIG. 5L shows an example module configured to couple to a connector. In this example, module 20 may have a substantially low profile over a skin surface. An example module 20 profile may be about 2 millimeters to about 15 millimeters; about 2 millimeters to about 4 millimeters; about 4 millimeters to about 6 millimeters; about 6 millimeters to about 8 millimeters; about 8 millimeters to about 10 millimeters; about 10 millimeters to about 12 millimeters; or about 12 millimeters to about 15 millimeters. The example module 20 may be connected to a connector 90. Such connectors 90 may be worn outside clothing and may be connected to one or more modules 20 that are worn inside the clothing. Thus, in some embodiments, module(s) 20 and connector 90 are on opposite sides of clothing 54 during use. The modules 20 may be designed such that they are not visible under clothing 154. Thus, device embodiments may be designed to be worn throughout the day including at work or while traveling. The light emitted by such modules 20 may be masked or shielded such that the light is not visible under clothing 154. In some embodiments, a module 20 includes one or more sensor and one or more light sources to provide light therapy to area region of interest on the body of a user. The thickness of module 20 embodiments may be reduced using design features and techniques, examples of which include but are not limited to: miniaturization of components, use of flexible and slim batteries, material selection, integrating one or more components with clothing, and modular designs. Details of such design features and techniques are described below. Connector 90 may comprise one or more module attachment sites for connecting one or more modules 20. Thus, connector 90 can act as a wearable device 10. In some embodiment, connector 90 is magnetic and can be magnetically attached to one or more modules 20. Examples of such embodiments include, but are not limited to: magnetic rings, magnetic buttons, flat magnetic surfaces, etc.

In some embodiments, connector 90 may act as a wearable device 10. The attachment of connector 90 to a module 20 may be detected and/or used as a switch to turn on or adjust one or more functions of module 20; examples of such functions (e.g. light delivery, sensing, etc.) are disclosed elsewhere in this specification. One advantage of this attachment 90 mechanism is that modules 20 can be secured to the user's typical clothing. In some embodiments, relative orientation of connector 90 and module 20 is detected. This detection may be achieved using one or more sensors, examples of which include, but are not limited to: magnetic sensors, electromagnetic sensors, proximity sensors, electrical sensors, motion-based sensors, etc. In some embodiments, the connector 90 may be turned or twisted relative to module 20 to achieve one or more actions; examples of which include, but are not limited to: turn on or off one of more functions of module 20, change one or more working parameters (e.g. therapy parameter(s), display parameter(s), user interface parameter(s), sensor parameter(s)) of module 20, etc. Thus, connector 90 may also act as a user interface and may include display portions, light indicators, or the like.

In any of the embodiments herein, a connection (e.g. a mechanical attachment, physical proximity, an electrical connection, a wireless connection, etc.) of one or more modules 20 with one or more wearable devices 10 or module attachment sites 14 or attachments 90 may be detected by one or more modules 20 and/or one or more wearable devices 10 and/or one or more module attachment sites 14 or attachments 90. This detection may be achieved using one or more sensors, examples of which include, but are not limited to: magnetic sensors, electromagnetic sensors, proximity sensors, electrical sensors, etc. This detection may be used to turn on one or more functions of module 20 when the connection is detected, turn off one or more functions of module 20 when the connection is undetectable, change one or more working parameters (e.g. therapy parameter(s), display parameter(s), user interface parameter(s), sensor parameter(s)) of module 20 responsive to detecting connection and/or disconnection of one or more module 20 from one or more attachment sites 14 and/or attachments 90.

Any of the devices and methods disclosed herein may be used to measure and/or track vital signs such as heart rate, skin or body temperature, or hydration levels. These measurements may be provided to the user at multiple time points or even continuously or in real-time data. These measurements may be made by sensors integrated into device 10 or modules 20. For example, thermal sensors may be integrated into device 10 or modules 20 for monitoring skin or dermal temperatures

Any of the devices and methods disclosed herein may be used for tracking metrics such as heart rate and body temperature during workouts. Such data may be sent to mobile apps for real-time feedback. Any of the devices and methods disclosed herein may be used for personalized health monitoring, such as sleep tracking or monitoring chronic conditions like cardiovascular diseases.

Any of the devices and methods disclosed herein that are used for women users may be “timed” to one or more phases of the user's menstrual cycle. Examples of such menstrual cycle timed methods include, but are not limited to: adjusting one or more treatment parameters such as dose across the menstrual cycle, initiating a therapy based on the menstrual cycle, stopping a therapy based on the menstrual cycle, adjusting a therapy based on symptoms that vary across the menstrual cycle, etc. Such menstrual cycle timed methods may be adjusted or modified manually by the user or through an automated mechanism wherein menstrual cycle data is obtained from the user and used in the menstrual cycle timed method. The menstrual cycle data may be obtained from wearables, through an app, through an electronic user interface, etc.

Any of the methods and devices disclosed herein may be used prior to initiating an activity that triggers pain or discomfort. Examples of such activities include, but are not limited to: sitting, standing, walking, running, jogging, cycling, engaging in an exercise, traveling, work-related activities, etc. This allows an “on demand” therapy that the user can use to reduce pain or discomfort from an activity that triggers pain or discomfort.

Any of the methods and devices disclosed herein may be used to tailor or personalize one or more therapies such that the therapy modules are in optimal location(s) and deliver optimized therapy. Methods and devices disclosed herein may incorporate closed-loop feedback. For example, the feedback obtained from delivered therapy may be used to change or modify subsequent therapy (e.g., by changing therapy parameters such as dose, therapy time, etc.). Such closed loop feedback may be performed in real time. The feedback obtained from delivered therapy may be obtained from one or more sensors 56. The feedback obtained from delivered therapy may also be provided through user inputs using an app or a user interface such as a touchscreen or buttons on modules 20. In some embodiments, the closed-loop feedback mechanism is entirely contained within a module 20. For example, the module 20 includes a processor that uses data from one or more sensors 56 to change one or more treatment parameters. In some embodiments, a wearable device 10 includes a sensor 56 that measures one or more of: nitric oxide data, microvasculature, and blood flow data. The data from sensor 56 is used for one or more of: determining an energy dose, creating closed-loop feedback, delivering optimal therapy, delivering personalized therapy, etc. The dose and other parameters of the therapy may change at one or more times based on the patient's physiological response.

FIG. 6A shows a flow diagram of an example computer-implemented method of delivering a therapy using a system that incorporates sensor feedback and modifies therapy parameters dynamically. Such methods may be used for personalized, real-time adjustments based on patient-specific details and sensor data to optimize therapeutic outcomes. At step 410, one or more initial inputs for configuring a wearable device 10 for a specific patient are received (i.e., individualizing the wearable device for a patient). These inputs may include one or more of:
1. Patient Information: This includes patient demographic details, health history, and specific conditions or diseases associated with the patient, etc.
2. Therapy Goals: The desired therapeutic outcomes (e.g., pain relief, muscle recovery, inflammation reduction, etc.)
3. System Configuration: Information regarding available one or more modules 20, their treatment parameters, etc.

At step 412, patient-specific initial therapy parameters are generated based on the inputs. These parameters are customized to the patient. These initial therapy parameters may include one or more of:
1. Personalization: The initial therapy parameters (e.g., dose, type, intensity, frequency, duration, etc.) may be specific to the patient's health condition, location of a module 20, goals, tolerance levels, etc.
2. Validation: The parameters may be reviewed and validated by a healthcare professional to ensure safety and efficacy.
3. Algorithmic Support: Algorithms or clinical guidelines may be used to generate the parameters based on the inputs.
4. Location of one or more modules on the patient to achieve a desired or predefined therapeutic effect.

At step 414, a placement of one or more modules 20 or their components (e.g., light sources 36, sensors 56, etc.) may be determined. For example, one or more sensor signals 56 may be received from one or more sensors to determine proper placement of one or more modules 20.

At step 416, initial therapy is caused to be delivered at initial, patient-specific parameters and monitoring data is received from sensors 56. Sensor 56 data (e.g., temperature, muscle contraction, pain levels, etc.) may be constantly, consistently, intermittently, on-demand, etc. monitored (e.g., in real time) to assess the therapy's performance. Direct patient feedback may be obtained by wearable device 10. One or more data integrity checks may be performed to determine that wearable device 10 is accurately capturing and storing data without interruption or errors.

At step 418, optionally, the method includes outputting therapy (e.g., current therapy parameters) and/or sensor data to a display. The display may be present on module 20 or on a separate device that communicated with a module 20. The display (e.g., a screen) may present actionable insights such as key insights or alerts (e.g., deviations from the expected progress) to help the user make one or more decisions. The user interface may be customizable to display only relevant metrics for the specific therapy being administered.

At step 420, the method includes receiving and analyzing sensor 56 data. Wearable device 10 may determine whether therapy adjustments should be performed. Wearable device 10 may use advanced algorithms, ML models, etc. to detect trends, patterns, or anomalies in sensor 56 data. Wearable device 10 may include predefined thresholds that trigger automatic or manual adjustments in one or more therapy parameters. Sensor 56 data may be processed continuously, intermittently, on-demand, etc. such that therapy parameter adjustments happen promptly.

At step 422, optionally, output one or more modified therapy parameters, based on sensor 56 data. Wearable device 10 (e.g., a processor or microcontroller within or electrically coupled to wearable device 10) may perform dynamic adjustments of one or more treatment parameters, examples of which are listed elsewhere herein based on sensor 56 feedback. Wearable device 10 may include one or more safety controls such as safety protocols to prevent harmful adjustments (e.g., exceeding intensity limits, exceeding dose limits, exceeding therapy time limits, etc.). Wearable device 10 may include one or more safety checks such that confirmation or approval from a healthcare professional is performed before implementation of the changes.

At step 424, the method may include causing one or more therapies to be delivered with modified parameters from step 422. The transition from initial therapy parameters to modified therapy parameters may be performed without interrupting a therapy session. Wearable device 10 (e.g., a processor or microcontroller within or electrically coupled to wearable device 10) may continue to monitor one or more patient's response(s) to the modified therapy parameters to ensure that the therapy remains safe and effective. If the modified therapy parameters cause adverse effect(s), wearable device 10 may use a reverting algorithm to revert to one or more previous therapy parameters or adjust the modified therapy parameters.

At step 426, wearable device 10 (e.g., a processor or microcontroller within or electrically coupled to wearable device 10) determines whether desired results have been achieved or whether the therapy has been completed. In some embodiments, the user may provide one or more inputs that are then used by the processor to determine whether desired results have been achieved. One or more predefined criteria may be stored in memory or in a database accessible by a processor of wearable device to determine therapy completion. Examples of such criteria include, but are not limited to: desired sensor 56 readings achieved, treatment time elapsed, treatment dose delivered, etc. The processor electrically coupled to the wearable device, the user, and/or healthcare providers may determine whether the desired treatment goals (e.g., pain reduction, muscle relaxation, etc.) have been met. If the desired results have not been achieved or if the therapy has not been completed, the method returns to step 420.

If the desired results have been achieved or if the therapy is complete, the method proceeds to step 428, where the therapy is stopped. Even after the therapy stops, wearable device 10 may continue post-therapy monitoring (e.g., using sensors 56) for a brief period to detect any delayed adverse effects. One or more treatment data and/or sensor 56 data may be securely stored, for example in memory or in a database (local or remote), for post-session analysis and/or record-keeping.

FIG. 6B shows a flow diagram of an example method of delivering a therapy using a system that incorporates sensor and user feedback and modifies therapy parameters dynamically. Such methods may be used for personalized, real-time adjustments based on patient-specific request or health indications, user feedback, and sensor data to optimize therapeutic outcomes. At step 410, the method includes receiving one or more initial inputs for configuring a wearable device 10 for a specific patient. These inputs may include one or more of:
1. Patient Information: This includes patient demographic details, health history, and specific conditions to be treated, etc.
2. Therapy Goals: The desired therapeutic outcomes (e.g., pain relief, muscle recovery, inflammation reduction, etc.)
3. System Configuration: Information regarding available one or more modules 20, their treatment parameters, etc.

At step 412, patient-specific initial therapy parameters are generated or determined based on the inputs. These parameters are customized to the patient. These initial therapy parameters may include one or more of:
1. Personalization: The initial therapy parameters (e.g., dose, type, intensity, frequency, duration, etc.) may be specific to the patient's health condition, goals, tolerance levels, etc.
2. Validation: The parameters may be reviewed and validated by a healthcare professional to ensure safety and efficacy.
3. Algorithmic Support: Algorithms or clinical guidelines may be used to generate the parameters based on the inputs.
4. Location of one or more modules on the patient to achieve a desired or predefined therapeutic effect.

At step 414, the method may include determining one or more modules 20 or their components (e.g., light sources 36, sensors 56, etc.) placement. The processor may receive one or more sensor 56 signals, for example, to determine proper placement of one or more modules 20.

At step 416, the method may include causing initial therapy to be delivered at initial, patient-specific parameters and, optionally, receiving monitoring data captured by sensors 56. Sensor 56 data (e.g., temperature, muscle contraction, pain levels, etc.) may be continuously, intermittently, on-demand, etc. monitored (e.g., in real time) to assess the therapy's performance. Direct patient feedback may be obtained by wearable device 10. One or more data integrity checks may be performed to determine that wearable device 10 is accurately capturing and storing data without interruption or errors.

At step 418, optionally, the method includes outputting to a display therapy (e.g., current therapy parameters) and/or sensor data. The display (e.g., a screen) may present actionable Insights such as key insights or alerts (e.g., deviations from the expected progress) to help the user take one or more decisions. The display may be customizable to display only relevant metrics for the specific therapy being administered.

At step 420, the method may include receiving and analyzing sensor 56 data and/or user input(s). Wearable device 10 (e.g., processor electrically coupled to the wearable device) may determine whether therapy adjustments should be performed. Wearable device 10 may use advanced algorithms, ML models, etc. to detect trends, patterns, or anomalies in sensor 56 data. Wearable device 10 may include predefined thresholds that trigger automatic or manual adjustments in one or more therapy parameters. Wearable device 10 may include a therapy interrupt feature to shut down a therapy based on user input(s) and/or sensor data. Sensor 56 data may be processed continuously, intermittently, on-demand, etc. such that therapy parameter adjustments occur promptly.

At step 422, optionally, wearable device 10 modifies one or more therapy parameters, based on sensor 56 data and/or optionally user input(s). User input(s) may be obtained from the patient or a healthcare provider. Wearable device 10 may perform dynamic adjustments of one or more treatment parameters, examples of which are listed elsewhere herein based on sensor 56 feedback. Wearable device 10 may include one or more safety controls such as safety protocols to prevent harmful adjustments (e.g., exceeding intensity limits, exceeding dose limits, exceeding therapy time limits, etc.).

At step 424, the method may include causing one or more therapies to be delivered with modified parameters from step 422. The transition from initial therapy parameters to modified therapy parameters may be performed without interrupting a therapy session. Wearable device 10 may continue to monitor one or more patient's response(s) to the modified therapy parameters to ensure that the therapy remains safe and effective. If the modified therapy parameters cause adverse effect(s), wearable device 10 may use a reverting algorithm to revert to previous therapy parameters or adjust the modified therapy parameters.

At step 430, wearable device 10 determines whether the therapy has been completed. Wearable device 10 may include one or more predefined criteria to determine therapy completion. Examples of such criteria include, but are not limited to: desired sensor 56 readings achieved, treatment time elapsed, treatment dose delivered, user input(s), etc.

If the therapy is complete, the method proceeds to step 436, where the therapy is stopped. Even after the therapy stops, wearable device 10 may continue post-therapy monitoring (e.g., using sensors 56) for a brief period to detect any delayed adverse effects. One or more treatment data and/or sensor 56 data may be securely stored (e.g., in memory or in a database) for post-session analysis and record-keeping.

If at step 430, it is determined that the therapy has not been completed, the method proceeds to step 432 to determine whether the user is satisfied with the therapy. At step 428, wearable device 10 determines whether the user is satisfied with the therapy progress or outcome. Wearable device 10 may actively solicit feedback from the patient to assess satisfaction with the therapy. Wearable device 10 may include one or more inputs or algorithms to collect objective or subjective satisfaction metrics. Such metrics include, but are not limited to: patient comfort, pain levels, or other relevant indicators. If the user is satisfied with the therapy, the method returns to step 420. If the user is not satisfied with the therapy, the method returns to step 434.

At step 434, one or more input(s) may be received at wearable device 10. These inputs may be obtained using a user interface on wearable device 10 or other means such as an application loaded on an electronic device. Examples of input(s) include, but are not limited to: user satisfaction, feedback to increase or decrease therapy power or intensity, feedback to increase or decrease therapy duration, feedback to change one or more therapy modes, feedback on meeting desired treatment goals (e.g., pain reduction, muscle relaxation, etc.), etc. Wearable device 10 may perform one or more checks to ensure that the input(s) do not compromise safety, especially if the modifications involve increased therapy dose or power or duration. After step 434, the method returns to step 420.

Any of the methods and device disclosed herein may include means for incorporating healthcare provider input(s) and/or delivering data to healthcare provider(s).

Although two method embodiments are described in FIGS. 6A and 6B, other embodiments may be utilized and derived therefrom. For example, embodiments of wearable device 10 may not include a display such that step 418 is not performed in method embodiments.

The methods may be performed by a processor (or one or more processors) electrically coupled to the module or the wearable device. The processor (e.g., central processing unit, digital signal processor, field-programmable gate array, application specific integrated circuit, etc.) may be in wearable device 10, in module 20, in a computing device 88 communicatively coupled to module 20 or wearable device 10, etc.

Any of the methods and devices disclosed herein may be used to treat fibromyalgia or other chronic pain conditions. Fibromyalgia may produce symptoms such as widespread pain and heightened sensitivity to stimuli like touch, pressure, and light. The pain may be felt across the body at sites called tender points or trigger points. Examples of such sites include but are not limited to: neck and shoulders: especially in the upper back and neck region, upper chest: especially around the sternum, elbows: especially in the outer area of the elbows, lower back, hips: especially in the buttocks, knees, and upper thighs. The pain points may vary in intensity and location across patients and across time points. One or more devices and methods disclosed herein may be used to treat such regions. The treatment locations and/or one or more treatment parameters may change across time points. One or more wearable devices 10 disclosed herein may be adjusted by the user such that one or more forces exerted by straps 12 or other patient contacting regions is sufficiently low to avoid causing sensitivity in fibromyalgia patients who have heightened sensitivity. Further, such straps 12 or other patient contacting regions may be flexible and soft to avoid causing pain or discomfort. Since the location(s) of pain/trigger points and the pain level(s) may change with time, the location(s) of one or more modules 20 and/or one or more treatment parameters may be adjusted accordingly. Embodiments of treatment systems may be designed that include one to 25 modules 20. PBM may be used in such patients for one or more of: reducing inflammation, improving circulation, and reducing pain. The starting dose or power level for such patients may be low initially and gradually increased based on the treatment effect (e.g., pain relief experienced by the patient) and occurrence of side effects. Combination therapies may be used. For example, one or more specific regions (e.g., pain points, trigger points) may be treated with one or more devices disclosed herein and a larger region of the body may be treated with other devices to achieve a combined effect.

A method for treating fibromyalgia based on the method shown in FIG. 6A is described below. At step 410, initial inputs are received into the system to configure a thin wearable device 10 (or e-textile) for the specific indications associated with the patient. Received data may include one or more of the following:
1. User Information: Demographic details, medical history, and specific conditions such as fibromyalgia-related symptoms, tender points, light sensitivity, etc.
2. Therapy Goals: The desired therapeutic outcomes are defined, which may include pain relief at specific trigger points, reduction of inflammation, improvement in muscle relaxation, etc.
3. System Configuration: Information related to the system's available modules 20 (e.g., flexible light sources embedded in the textile, sensors, and treatment settings or parameters) may be entered for customized therapy delivery.

At step 412, patient-specific initial therapy parameters are generated based on inputs at step 410 to provide personalized treatment. These parameters may be tailored to the user's fibromyalgia condition, sensitivity, tolerance levels, etc. One or more therapy parameters, including but not limited to: light wavelength(s), dose, intensity, duration, etc. may be customized. Optionally, a healthcare professional may review and validate the therapy parameters to ensure safety and efficacy, especially considering the patient's tolerance for light therapy or physical pressure. The system may utilize clinical algorithms or guidelines to optimize the parameters, particularly for fibromyalgia-specific therapies targeting multiple pain points.

At step 414, one or more wearable devices 10 are placed at the appropriate body locations. Such locations may include specific fibromyalgia pain points, such as the neck, lower back, or knees. Sensors 56 in the device(s) may be used to monitor mechanical fitting to ensure proper contact with the patient's skin without causing discomfort. The placement of one or more device components may be adjusted dynamically to ensure effective targeting of pain regions without aggravating pressure-sensitive areas.

At step 416, initial therapy is delivered based on the patient-specific therapy parameters. Throughout the therapy session, data captured by sensors 56 may be used to monitor real-time responses. This monitoring may include:
1. Pain Levels: Feedback on pain reduction or muscle relaxation may be captured from one or more sensors 56 or directly from the patient.
2. Light Sensitivity: The system may monitor the user's tolerance to light intensity and adjust one or more therapy parameters.
3. Data Integrity: Continuous checks may be performed to ensure that sensor 56 readings are accurate and uninterrupted, ensuring that the therapy is effective and safe.

At step 418, the system's user interface, accessible through a connected app or screen, may be used to display real-time therapy data and actionable insights. The interface may be used to show key metrics such as therapy duration, therapy time remaining, intensity, and patient feedback, customized for fibromyalgia treatment. Alerts or deviations from expected outcomes (e.g., no reduction in pain) may be flagged to the patient or healthcare provider to help guide therapy adjustments.

At step 420, sensor 56 data is analyzed. The system may assess whether adjustments to treatment parameters are to be performed based on trends or anomalies in sensor feedback, such as variations in pain levels or skin sensitivity. Advanced algorithms or machine learning models may be used to detect patterns in the sensor 56 data and/or indicate whether therapy adjustments are to be made to improve targeting of fibromyalgia pain points. Predefined thresholds may trigger automatic or manual modifications to therapy parameters (e.g., reducing light intensity or increasing treatment duration) to optimize outcomes.

At step 422, the system may dynamically adjust therapy parameters in response to sensor 56 feedback, to ensure continuous comfort and effectiveness. One or more device components may include programmed limits to ensures that treatment(s) and treatment modification(s) remain within safe limits (e.g., light intensity or therapy duration) and may require approval of a healthcare provider for any significant adjustments. Therapy adjustments, such as reducing light intensity for sensitive patients or increasing therapy duration for more severe pain, may be made without interrupting the therapy.

At step 424, the system continues to deliver therapy with the adjusted parameters. The user's response to the modified therapy parameters may be monitored continuously to ensure the safety and efficacy of the treatment. If the modifications cause discomfort or adverse effects, the device may revert to a previous setting or further adjust therapy parameters.

At step 426, the device checks if the therapy has achieved the desired results, such as pain relief or reduction in muscle tension, or has completed using predefined criteria. A therapy may end when sensor readings or other data indicate that the patient has reached their therapeutic goals, such as a decrease in pain levels, improvement in muscle relaxation, a treatment dose, etc. After the therapy session concludes, the device may continue monitoring the patient's condition to detect any delayed effects, such as skin irritation or residual pain.

At step 428, once the therapy session is complete, the device stops the therapy and securely stores all relevant data for post-session analysis. This data may be reviewed by the patient or healthcare provider to assess an effectiveness of a treatment and/or session and adjust future therapy plans.

FIG. 7A shows a flow diagram of an example method of using machine learning (ML) and artificial intelligence (AI) to deliver therapy. This embodiment combines various steps such as data collection, medical imaging, data analysis, and therapy control based on patient-specific parameters such as pain location(s) and pain sources. Any of the AI/ML based methods described herein may dynamically optimize treatment parameters using one or more of: pre-existing data, real-time feedback, and image analysis.

At step 110, the method may include receiving patient data. The data may be fed or otherwise entered into device 10. Patient data may include one or more of: medical history, pain locations, symptoms, and therapy requirements. The data may provide the foundational context for AI-based therapy customization. Inputted patient data may be used to define or create an initial treatment protocol. Patient data may be obtained from sources including, but not limited to:

Structured input forms through which healthcare providers provide data and/or interfaces with Electronic Medical Records (EMR). For example, patient data may be processed in a variety of ways. For example demographic and clinical data (e.g., age, sex, weight, comorbidities, prior treatments, allergies, and lifestyle factors, etc.) may be converted to structured tabular data. Diagnostic data such as laboratory results, electrophysiological recordings, or biomechanical measurements, etc. may be time-series data processed through filtering, normalization, and statistical feature extraction.

At step 112, the method may include receiving imaging data, for example, from a diagnostic imaging procedure (e.g., MRI, CT scan, X-ray, etc.). An AI model may be trained using the imaging data to identify disease locations, pain generating locations, therapy-relevant areas, target areas for delivering therapy, etc. The imaging may be performed using imaging equipment that is compatible with and has a data transfer interface with an AI system.

At step 114, the imaging data is analyzed. The imaging data may be securely transferred to an AI/ML-based system for further analysis. The AI model may continuously learn from incoming data, thereby enhancing its ability to detect patterns and optimize therapy suggestions. The imaging data may undergo preprocessing steps such as: spatial normalization to anatomical templates, noise reduction and artifact correction, and segmentation of relevant anatomical structures using pre-trained convolutional neural networks (CNNs) or other methods. The imaging data may be processed to identify bodily areas for carrying out PBM interventions. Secure data transmission channels between imaging device(s) and the AI system or other processing systems may be used to transmit data. The processing system(s) may have a sufficient processing power to handle large datasets and enable real-time responsiveness.

At step 116, one or more areas of pain locations are determined from image analysis. The AI model analyzes the imaging data to pinpoint specific areas of pain. The AI model may be used for one or more of: distinguishing between normal and abnormal tissues, categorizing tissues, assessing severity of a condition, supporting personalized treatment planning, determining precise areas of pain, etc. This step may be performed using one or more pre-trained AI models capable of detecting patterns in medical images. Robust datasets may be used to support model training and continuous improvement. The architecture of any of the AI models mentioned herein may employ one or more of:
1. Multimodal Fusion Networks such as Branch Networks with separate subnetworks for each input modality (e.g., ResNet-based CNN for imaging, Transformer-based encoder for time-series, fully connected layers for tabular data), Fusion Layer with concatenation or attention-based fusion of learned feature vectors from each branch, etc.
2. Core Decision Networks which may include a. Transformer encoder-decoder layers for cross-modal attention, enabling the model to correlate anatomical features with diagnostic measurements or b. Dense layers for regression (continuous therapy parameters) and classification (categorical therapy location zones).
3. Output Layer which may generate quantitative therapy parameters (e.g., dosage, duration, power/energy levels, wavelength(s)) and spatial coordinates or region maps for therapy application.

Any AI models mentioned herein may be trained using one or more methods; examples of which include, but are not limited to:
1. Data Labeling & Ground Truth: Data from clinical trials, patients'experience records, clinical use records, annotated records, etc. may be used to generate labels of location(s) and/or one or more working parameters for modules 20.
2. Supervised Learning:
3. Semi-Supervised and Transfer Learning
4. Reinforcement Learning: Optimal parameters may be selected by simulating therapy outcomes within a patient-specific model.
5. Federated Learning: Model training may be distributed across multiple centers without transferring raw patient data, ensuring compliance with privacy regulations.

Any trained AI model mentioned herein may be deployed via: Edge Deployment, Cloud Deployment, or hybrid deployment. Edge Deployment may be optimized lightweight models (e.g., via model pruning, quantization) for real-time operation on devices such s modules 20, computing device 88, etc. Cloud Deployment may be used for full-scale model inference which may utilize computationally intensive fusion architectures and is connected to such architectures. Hybrid deployment may include local pre-processing and inference for urgent tasks and processing on cloud servers for advanced decision-making.

Examples of algorithms that may be used to build any of the models mentioned herein include, but are not limited to: Convolutional Neural Networks (CNNs), Vision Transformers (ViTs), Recurrent Neural Networks (RNNs) or Transformers, Gradient Boosted Decision Trees (GBDTs), and Attention-based Multimodal Transformers.

At step 118, initial location(s) of modules 20 are generated. This may be done using trained AI/ML models and identified pain locations at step 116. The AI model may recommend optimal placement sites for one or more modules 20 to maximize therapeutic efficacy. This may be done using algorithms capable of generating module 20 placement suggestions. The AI model may use configurable parameters to adjust recommendations for individual patients.

At step 120, one or more treatment parameters are generated by an AI/ML model. The AI/ML model may use inputs including, but not limited to: pain location(s), user inputs, and real-time data. Examples of treatment parameters (e.g., intensity, frequency, dose) are given elsewhere in this specification. Treatment parameters may be selected based on factors such as type of therapy, real-time inputs, pain locations, etc. One or more treatment parameters may be refined dynamically for responsive therapy.

At step 122, one or more modules 20 are selected based on pain location(s), therapy parameters, etc. Examples of modules 20 are disclosed elsewhere herein.

At step 124, one or more treatment parameters are set or adjusted in modules 20 or other portions of device 10. This setting or adjustment may be done automatically or manually. Device 10, modules 20, etc. may adjust one or more treatment parameters (e.g., intensity, duration, etc.) in real time based on factors such as patient feedback (e.g., through an app, through a user interface, etc.), symptom feedback, and parameters generated in step 120. This dynamic adjustment may be used to optimize therapy effectiveness, safety, and/or comfort. Such adaptive treatment protocols may be used in any of the methods and devices disclosed herein. One or more device 10 components may include functionalities including, but not limited to: Real-time control capability for adjusting one or more treatment parameters (e.g., based on based on physiological responses),

Secure storage for storing parameters or settings for future therapy sessions, Continuous real-time data input mechanism(s) from sensors or other monitoring systems, and Adaptive AI model(s) that adjust one or more parameters in response to one or more inputs.

At step 126, location(s) of one or more modules 20 on the patient are determined or adjusted. Such locations may be determined based on the designs of one or more of: wearable device 10, strap 12 or other attachment mechanisms, module attachment sites 14, etc.

At step 128, one or more modules 20 are connected to a wearable device 10 based on desired module 20 locations. Examples of wearable devices 10 are disclosed elsewhere herein. The user than wears device 10 such that one or more modules 20 contact the user.

At step 130, one or more therapies are delivered using one or more modules 20 as per the parameters determined in step 124. Device 10 may include one or more mechanisms (e.g., sensors or feedback loops) to ensure that the therapies are delivered at precise treatment parameters. Device 10 may include one or more safety mechanisms to prevent overtreatment or undertreatment.

At step 132, the system or user determines whether the therapy location(s) are satisfactory. When the user determines that the therapy location(s) are satisfactory, step 134 is performed. When the user determines that the therapy location(s) are unsatisfactory, the method returns to step 126 wherein location(s) of one or more modules 20 on the patient are determined or adjusted 134.

At step 134, the system or user determines whether the therapy parameter(s) are satisfactory. When the user determines that the therapy parameters(s) are satisfactory, step 130 is performed. When the user determines that the therapy parameters(s) are unsatisfactory (e.g., due to lack of treatment effect, discomfort, etc.), the method returns to step 124 such that one or more treatment parameters are set or adjusted in modules 20 or other portions of device 10.

FIG. 7B shows a flow diagram of an example method employing ML and AI for delivering therapy targeting endometriosis and other pelvic pain conditions. This embodiment initiates with the collection of clinical data and imaging analysis to detect endometriosis lesions, followed by AI-guided module placement and real-time therapy adjustments to ensure a personalized approach. AI/ML-based techniques may be used to identify endometriosis sites or pain generating regions and guide subsequent therapy.

At step 212, one or more diagnostic tests are performed. Diagnostic tests include imaging tests and non-imaging tests. Examples of imaging tests include, but are not limited to: MRI, CT scanning, X-ray imaging, hysteroscopy, laparoscopy, ultrasound-based imaging (e.g., transvaginal ultrasonography, abdominal ultrasonography, gel sonovaginography, doppler sonography with or without uterine artery resistance index, color doppler ultrasonography, spectral doppler ultrasonography, transrectal ultrasonography, saline-infusion sonography, contrast ultrasonography), etc. The imaging may be performed using imaging equipment that is compatible with, and has a data transfer interface with, an AI system. One or more AI models may be trained with the imaging data to identify disease locations, pain generating locations, therapy-relevant areas, target areas for delivering therapy, etc. Examples of non-imaging tests include, but are not limited to: AI-based analysis of the patient's symptoms, blood or urine tests, menstrual blood-based analysis, biopsy-based analysis, etc.

At step 214, the test data is analyzed. Imaging and non-imaging data may be securely transferred to an AI/ML-based system for further analysis. The AI model may continuously learn from incoming data, thereby enhancing its ability to detect patterns and optimize therapy suggestions. The imaging data may be processed to identify bodily areas in which to perform PBM interventions. Secure data transmission channels between imaging device(s) and the AI system or other processing systems may be used to transmit data. The processing system(s) may have a sufficient processing power to handle large datasets and enable real-time responsiveness.

At step 216, presence and/or one or more location(s) of endometriosis or endometriosis-related pain are determined from data analysis. One or more AI models may be used to analyze the test data to pinpoint specific areas of pain. One or more AI models may be used for one or more of: distinguishing between normal and abnormal tissues, categorizing tissues, assessing presence/severity of endometriosis, supporting personalized treatment planning, determining precise areas of pain, etc. This step may be performed using pre-trained AI models capable of detecting patterns in medical images and other data. Robust datasets may be used to support model training and continuous improvement. One or more AI models may be used for analyzing multimodal data.

At step 218, initial location(s) of modules 20 are generated or determined. This may be done using trained AI/ML models and inputs such as identified endometriosis and/or pain locations at step 216, anatomical markers, etc. The AI model may recommend optimal placement sites for one or more modules 20 to maximize therapeutic efficacy. This may be done using algorithms capable of generating module 20 placement suggestions. The AI model may use configurable parameters to adjust recommendations for individual patients.

At step 220, one or more treatment parameters are generated by an AI/ML model. The AI/ML model may use inputs including, but not limited to: endometriosis location(s), pain location(s), diagnostic test result(s), user inputs, and real-time data. Examples of treatment parameters (e.g., intensity, frequency, dose) are given elsewhere herein. Treatment parameters may be selected based on factors such as type of therapy, real-time inputs, endometriosis location(s), pain locations, etc. One or more treatment parameters may be refined dynamically for responsive therapy.

At step 222, one or more modules 20 are selected based on factors such as endometriosis location(s), pain location(s), therapy parameters, patient's anatomy, etc. Examples of modules 20 are disclosed elsewhere herein.

At step 224 one or more treatment parameters are set or adjusted in modules 20 or other portions of device 10. This setting or adjustment may be done automatically or manually. Device 10, modules 20, etc. may adjust one or more treatment parameters (e.g., intensity, duration, etc.) in real time based on factors such as patient feedback and parameters generated in step 120. This dynamic adjustment may be used to optimize therapy effectiveness, safety, and comfort. One or more device 10 components may include functionalities including, but not limited to: Real-time control capability for adjusting one or more treatment parameters (e.g., based on based on physiological responses), Secure storage for storing parameters or settings for future therapy sessions, Continuous real-time data input mechanism from sensors or other monitoring systems, and Adaptive AI model(s) that adjust one or more parameters in response to one or more inputs.

At step 226, location(s) of one or more modules 20 on the patient are determined or adjusted. Such locations may be determined based on the designs of one or more of: wearable device 10, strap 12 or other attachment mechanisms, module attachment sites 14, etc.

At step 228, one or more modules 20 are connected to a wearable device 10 based on desired module 20 locations. Examples of wearable devices 10 are disclosed elsewhere in this specification. The user than wears device 10 such that one or more modules 20 are positioned to deliver therapy to the user.

At step 230, one or more therapies are delivered using one or more modules 20 as per the parameters decided in step 124. Device 10 may include one or more mechanisms (e.g., sensors or feedback loops) to ensure that the therapies are delivered at precise treatment parameters. Device 10 may include one or more safety mechanisms or algorithms to prevent overtreatment or undertreatment.

At step 232, the user determines whether the therapy location(s) are satisfactory. When the user determines that the therapy location(s) are satisfactory, step 134 is performed. When the user determines that the therapy location(s) are unsatisfactory, the method returns to step 126 such that location(s) of one or more modules 20 on the patient are determined or adjusted 134.

At step 234, the user determines whether the therapy parameter(s) are satisfactory. When the user determines that the therapy parameters(s) are satisfactory, step 130 is performed. When the user determines that the therapy parameters(s) are unsatisfactory (e.g., due to lack of treatment effect, discomfort, etc.), the method returns to step 124 such that one or more treatment parameters are set or adjusted in modules 20 or other portions of device 10.

FIG. 7C shows a flow diagram of an example method employing ML and AI for delivering therapy targeting back pain and related conditions. This embodiment initiates with the collection of clinical data and/or imaging analysis to detect lesions in the spine or surrounding anatomy, followed by AI-guided module placement and real-time therapy adjustments to ensure a personalized approach. AI/ML-based techniques may be used to identify pathology and/or pain generating regions and guide subsequent therapy.

At step 312, one or more imaging tests are performed. Examples of imaging tests include, but are not limited to: MRI, CT scanning, X-ray imaging, invasive tests including injecting pain generating agents under imaging guidance, invasive tests including injecting pain lowering agents (e.g., anesthetics) under imaging guidance, ultrasound-based imaging, etc. The imaging may be performed using imaging equipment that is compatible with, and has a data transfer interface with, an AI system. One or more AI models may use the imaging data to identify disease locations, pain generating locations, therapy-relevant areas, target areas for delivering therapy, etc.

At step 314, the test data is analyzed. Imaging and non-imaging data may be securely transferred to an AI/ML-based system for further analysis. The AI model may continuously learn from incoming data, thereby enhancing its ability to detect patterns and optimize therapy suggestions. The imaging data may be processed to identify bodily areas in which to perform PBM intervention. Secure data transmission channels between imaging device(s) and the AI system or other processing systems may be used to transmit data. The processing system(s) may have a sufficient processing power to handle large datasets and enable real-time responsiveness.

At step 316, one or more location(s) of back pain are determined from data analysis. One or more AI models may be used to analyze the test data to pinpoint specific areas of pain. One or more AI models may be used for one or more of: distinguishing between normal and abnormal tissues, categorizing tissues, assessing presence/severity of back pain, supporting personalized treatment planning, determining precise areas of pain, etc. This step may be performed using pre-trained AI models capable of detecting patterns in medical images and other data. Robust datasets may be used to support model training and continuous improvement. One or more AI models may be used for analyzing multimodal data.

At step 318, initial location(s) of modules 20 are generated or determined. This may be done using trained AI/ML models and inputs such as identified back pain locations at step 316, anatomical markers, etc. The AI model may recommend optimal placement sites for one or more modules 20 to maximize therapeutic efficacy. This may be done using algorithms capable of generating module 20 placement suggestions. The AI model may use configurable parameters to adjust recommendations for individual patients.

At step 320, one or more treatment parameters are generated by an AI/ML model. The AI/ML model may use inputs including, but not limited to: back pain location(s), diagnostic test result(s), user inputs, and real-time data. Examples of treatment parameters (e.g., intensity, frequency, dose) are given elsewhere herein. Treatment parameters may be selected based on factors such as type of therapy, real-time inputs, back pain locations, etc. One or more treatment parameters may be refined dynamically for responsive therapy.

At step 322, one or more modules 20 are selected based on factors such as back pain location(s), therapy parameters, anatomical markers, patient's anatomy, etc. Examples of modules 20 are disclosed elsewhere herein.

At step 324 one or more treatment parameters are set or adjusted in modules 20 or other portions of device 10. This setting or adjustment may be done automatically or manually. Device 10, modules 20, etc. may adjust one or more treatment parameters (e.g., intensity, duration, etc.) in real time based on factors such as patient feedback and parameters generated in step 120. This dynamic adjustment may be used to optimize therapy effectiveness, safety, and comfort. One or more device 10 components may include functionalities including, but not limited to: Real-time control capability for adjusting one or more treatment parameters (e.g., based on based on physiological responses), Secure storage for storing parameters or settings for future therapy sessions, Continuous real-time data input mechanism from sensors or other monitoring systems, and Adaptive AI model(s) that adjust one or more parameters in response to one or more inputs.

At step 326, location(s) of one or more modules 20 on the patient are determined or adjusted. Such locations may be determined based on the designs of one or more of: wearable device 10, strap 12 or other attachment mechanisms, module attachment sites 14, etc.

At step 328, one or more modules 20 are connected to a wearable device 10 based on desired module 20 locations. Examples of wearable devices 10 are disclosed elsewhere in this specification. The user than wears device 10 such that one or more modules 20 are positioned to deliver therapy to the user.

At step 330, one or more therapies are delivered via one or more modules 20 as per the parameters decided in step 124. Device 10 may include one or more mechanisms (e.g., sensors or feedback loops) to ensure that the therapies are delivered at precise treatment parameters. Device 10 may include one or more safety mechanisms to prevent overtreatment or undertreatment.

At step 332, the system or user determines whether the therapy location(s) are satisfactory. When the user determines that the therapy location(s) are satisfactory, step 134 is performed. When the user determines that the therapy location(s) are unsatisfactory, the method returns to step 126 such that location(s) of one or more modules 20 on the patient are determined or adjusted 134.

At step 334, the user determines whether the therapy parameter(s) are satisfactory. When the user determines that the therapy parameters(s) are satisfactory, step 130 is performed. When the user determines that the therapy parameters(s) are unsatisfactory (e.g., due to lack of treatment effect, discomfort, etc.), the method returns to step 124 such that one or more treatment parameters are set or adjusted in modules 20 or other portions of device 10.

The brain-gut axis (also called the gut-brain axis) is the neural connection between the central nervous system (CNS) and the enteric nervous system (ENS). It enables a bidirectional connection between the brain and the gastrointestinal tract. This brain-gut axis, includes the brain, the spinal cord, the autonomic nervous system (sympathetic nervous system, parasympathetic nervous system, and ENS), and the hypothalamic-pituitary-adrenal (HPA) axis. The brain affects several physiological activities through both vagal (neural) and HPA axis (hormonal) communication. Some embodiments herein may be used for influencing this communication. Further, some bodily cells and tissues are influenced by the gut microbiota. Some embodiments described herein may be used for influencing the gut microbiota by affecting the brain-gut axis locally (e.g. by influencing intestinal cells and ENS), but also by directly influencing neuroendocrine and metabolic functions. Influencing the gut microbiota may be used to alleviate anxiety and/or depressive-conditions.

FIG. 8A is a diagram showing the interaction of the gut and the brain and some bodily regions where one or more therapies disclosed herein may be delivered. One or more therapies disclosed herein may be delivered singly or in combination with other therapies (e.g. one or more therapies disclosed herein) at locations including, but not limited to: brain cortex (A), brain base including the cerebellum (B), regions around the upper vagus nerve (C), regions around the lower vagus nerve (C), the large intestine and region around the large intestine (E), and the small intestine and region around the small intestine (F). For example, two or more PBM therapies may be delivered at two or more separate locations. The PBM therapies may be delivered from the same device 10 or multiple devices 10. The PBM therapies may be delivered utilizing a single modules 20 or multiple modules 20. The PBM therapies may be delivered utilizing one or more sensors 56 to trigger modes of the modules, and/or therapy timing or parameters. In some embodiments, one or more PBM therapies may be combined with an electrical stimulation therapy. Similarly, permutations of one or more therapies disclosed herein are possible based on the time (simultaneous, before, or after) of delivery of the one or more therapies.

One or more therapies described herein may be used to affect mechanoreceptors, gut peptides, neurotransmitters, microbial metabolites, regulation of CRF and CRF1 receptors, activation of the Crh signaling pathway in the limbic system, neurotransmitters (examples include, but are not limited to: serotonin, acetylcholine, dopamine, and norepinephrine), vagus nerve functioning, etc.

FIGS. 8B-8Q show a variety of devices and methods to deliver one or more therapies that can affect the functioning of the gut-brain axis. In such methods and devices, a variety of sensing methods and devices may be used to perform one or more functions e.g. obtaining feedback, determining a biomarker level, and other functions disclosed elsewhere in this specification. In some embodiments, ECG (electrocardiogram) and/or EEG (electroencephalogram) and/or other electrical measurements are used. In some embodiments, between one to 20 electrodes are used for such sensing. Imaging data such as X-ray imaging, CT scans, MRI, ultrasound imaging, etc. may be used to determine the placements and/or working parameters of one or more devices disclosed herein. Functional feedback may be obtained and used in any of the methods and devices disclosed herein. In some embodiments, functional near-infrared spectroscopy using optical solutions may be used to monitor the brain activity. Examples of such methods include, but are not limited to: fNIRS and bbNIRS. Sensors or other elements for obtaining such functional feedback may be present on one or more modules disclosed herein. Thus, PBM and functional feedback may work simultaneously in suitable embodiments e.g. body-worn devices.

The devices described herein may include and/or otherwise use fNIRs and/or bbNIRs. In methods using fNIRS, one or more discrete wavelengths may be used for sensing and/or monitoring. In methods using bbNIRS, a broad spectrum of light may be used for sensing and/or monitoring. In some embodiments using bbNIRS, one or more broadband light sources (e.g. quartz lamps) may be used. In some embodiments using bbNIRS, one or more phosphor-based LEDs or lasers may be used to generate white light or other broadband lights. For example, a broadband near-infrared radiator such as an LED or laser source may be combined with phosphor or other materials to produce a broadband light source that fills one or more spectra continuously instead of discreetly. Multiple wavelengths or spectral bands may be measured to obtain more details about multiple functional aspects of the brain and other regions. Sensors or other elements may be optically or electrically filtered to analyze various spectral regions. One or more analyzes may be performed by combining one or more of: data at one or more wavelengths, data collected at one or more time points, and combinations thereof.

Analyses in time domain may be used to separate source and signal measurements (e.g. using filtered detectors) to detect and/or measure specific parameters, biomarkers, etc. Other methods may be used to separate source and signal measurements. For example, an excitation light source may be turned on, modulated, and then turned off. The sensing may be performed when the source is turned off. One or more digital processing techniques may be used to increase the signal-to-noise ratios of any of the measurements herein.

PBM may be delivered through one or more optodes that combine illumination and sensing functions. The illumination source may emit light of one or more wavelengths that is incident on one or more bodily regions. The light interacts with the bodily regions such that one or more optical characteristics (e.g., fluorescence intensity, absorbance, or luminescence lifetime) of the light are changed through processes such as anisotropic scattering in the brain. A detector measures these changes in optical properties of by measuring one or more of: amount of light absorbed, fluorescence or luminescence intensity, the change in the luminescence lifetime, etc. The detector generates electrical signals that are then processed. In some embodiments, LEDs that are reverse polarized are used as sensors. Such LEDs may use methods such as time domain spectroscopy to perform one or more measurements.

FIGS. 8B-8F show various embodiments of devices for delivering therapy to the head. Wearable devices 10 for placing therapy modules 20 on the head may be designed to ensure precision, comfort, and repeatability during treatments. These devices may vary in design and functionality based on the therapy type and target regions. Some examples of such wearables include, but are not limited to:
1. Headbands worn around the forehead or across the scalp. One embodiment shown in FIG. 8B. Multiple such headbands may be combined during use.
2. Caps and helmets which include one or more module attachment sites 14. One embodiment is shown in FIG. 8C. These and other body-worn wearable devices 10 may include LEDs or some other indicator of the placement of sensors 20. Such caps and helmets may include adjusting mechanisms (e.g. straps) to adjust for different head sizes.
3. Adjustable straps that are designed to be positioned over specific scalp regions.
4. Adhesive-based wearables that are single-use or reusable and are placed and attached directly on the scalp or hair using adhesives. Such wearables may also include alternative mechanisms such as vacuum-based adhesion.
5. Wearables positioned on the ear (example shown in FIG. 8D), nose, or mouth. For example, wearables embodiments (e.g., bands, earbuds, ear clips) may be designed to attach to the ear to target the auricular branch of the vagus nerve.
6. Full-head nets (example shown in FIG. 8E) that are stretchable. Such nets may cover the entire scalp for comprehensive treatment or monitoring.
7. Smart glasses or visors. Such embodiments are especially suited to deliver therapy to the forehead and upper brain areas.
8. Soft, moldable headgear that conforms to the head's shape.
9. Slim devices designed to be inserted through natural or artificially created openings in the body. Such devices could be inserted using minimally invasive or non-invasive techniques through the patient's natural or medically created orifices. Such devices could be inserted into patient's natural or medically created cavities. Such devices could be in the form of catheters or other surgical tools and may also be self-inserted by the user, One such embodiment is shown in FIG. 8F.
10. Combination wearables that combine one or more wearables described herein.

FIGS. 8G and 8H show two embodiments of circumferential locations of modules 20 around the head. One or more bioinformatics data may be used to determine one or more parameters of the methods and devices disclosed herein. For example, bioinformatics data may be used to determine and/or guide the placement of one or more modules 20. Examples of bioinformatics data include, but are not limited to: gene signaling pathways, protein expressions, blood biomarkers, imaging data, pathology data, etc. In some embodiments wherein PBM or other therapies are delivered to the brain, the suggested or optimal head placements of one or more modules 20 are overlaid on an image (e.g. electronic image on a screen, a paper print, etc.) of the scalp to guide the user or a physician on the optimal geographic location of one or more modules 20 on the scalp. In some embodiments of treating some conditions (e.g. behavioral pathologies such as ADHD, Parkinson's disease, anxiety, fibromyalgia, etc.) specific portions of the brain may be targeted using sensors 20 positioned on specific locations of the scalp. Bioinformatics data may be used to generate a protein targeting system wherein the data is used to analyze the proteins (e.g. TNF alpha) and other molecules that are influencing behavioral pathologies such as ADHD and calculate the impact of PBM on such proteins one or more other molecules and design treatment protocols. Thus, one or more brain treatments may be designed that target specific proteins and other molecules on one or more specific locations of the brain using PBM. As disclosed elsewhere herein, the effect of the delivered PBM therapy may be measured and used for adjusting one or more working parameters, closed-loop feedback, etc. In some embodiments of treating some conditions (e.g. neurodegenerative pathologies such as Alzheimer's disease), the entire brain or larger, nonspecific portions of the brain may be targeted.

One or more anatomical landmarks of the head may be used to guide the placement of one or more modules 20 disclosed herein. Such anatomical landmarks may be based on one or more factors including, but not limited to: the relationship between module 20 location and the underlying area of the cerebral cortex, skull thickness at the location, presence/absence of hair. location of a therapeutic target region of the brain, etc. Placement locations of one or more modules 20 may also be defined in terms of standardized locations. One advantage of using standardized locations is increased consistency of module 20 placement between therapy sessions. In some embodiments, these placement locations may be defined by 10-20 or 10-10 system of electroencephalography (EEG) electrode placement. However, other systems of defining placement locations may also be used.

The 10-20 system is a standardized method for placing electrodes on the scalp for EEG. It may be used to achieve consistent, reproducible positioning of modules 20, sensors 56 or other structures based on the relationship between the scalp and underlying brain structures. The system divides the head into proportional distances of 10% or 20% of the total length between anatomical landmarks, such as the nasion (bridge of the nose), inion (bump on the back of the head), and two preauricular points. Letters and numbers of the 10-20 nomenclature may be used to define placement locations on the scalp. Letters in this system represent the brain regions they cover: F for frontal, T for temporal, C for central, P for parietal, and O for occipital, with Z indicating the midline. Odd numbers (e.g., F3, T5) are used for electrodes on the left hemisphere, while even numbers (e.g., F4, T6) are used for the right hemisphere. Electrodes along the midline, like Cz or Fz, use only letters and “Z” without numbers. The 10-10 system is an extension of the 10-20 system, with higher number of placement locations. In this system, intermediate electrode positions are added between standard 10-20 locations, dividing distances into 10% increments. This system includes additional labels such as AF (anterior frontal), FC (frontal-central), CP (central-parietal), and PO (parietal-occipital), along with numbers or letters for lateralization. Like the 10-20 system, odd numbers are assigned to the left hemisphere, even numbers to the right hemisphere, and “Z” indicates midline locations. The increased density of placement locations of the 10-10 system may be used for one or more of: more detailed mapping of brain activity, more detailed mapping of data from one or more sensors 56, improved accuracy in delivering one or more therapies, etc. Other higher resolution systems such as the 10-5 system may also be used.

FIGS. 8I-8J show example placement locations of one or more modules relative to a head of a patient. In these examples, the 10-20 system of EEG electrode placement is used to describe module 20 locations.

FIG. 8I shows an embodiment of placement locations of six modules 20 for delivering one or more therapies to patients with Alzheimer's disease. The module placement sites are circled. As shown in FIG. 8I, such modules 20 may be placed at T5, T6, P3, P4, Pz, and Oz (not shown) locations. The placements shown may be used to target the functioning of Memory & Default Mode Network (DMN). Module 20 placements shown may be used to delivery energy or other therapies to bilateral temporal/parietal & posterior DMN hub.

FIG. 8J shows an embodiment of placement locations of four modules 20 for delivering one or more therapies to patients with ischemic stroke. As shown in FIG. 8J, such modules 20 may be placed at C3, C4, F3, and F4 locations. An additional placement at FCz may be used. The placements shown may be used to target the functioning of one or more of: motor Cortex for motor recovery, frontal cortex for attention and planning deficits, and supplementary motor area. Module 20 placements shown may be used to delivery energy or other therapies to lesion site(s) and contralateral homolog.

FIGS. 8K-8L show example placement locations of one or more PBM modules relative to the head of a patient. In these examples, the 10-10 system of EEG electrode placement is used to describe module 20 locations.

FIG. 8K shows an embodiment of placement locations of two to three modules 20 for delivering one or more therapies to patients with Major Depressive Disorder (MDD). As shown in FIG. 8K, such modules 20 may be placed at F3 and Fpz locations with an optional placement at F4 location. The placements shown may be used to target the functioning of Left Dorsolateral Prefrontal region for balance interhemispheric activity and the midline region for limbic and mood regulation. Module 20 placements shown may be used to delivery energy or other therapies to left and/or right dorsolateral prefrontal cortex (DLPFC).

FIG. 8L shows an embodiment of placement locations of four modules 20 for delivering one or more therapies to patients with Traumatic Brain Injury (TBI). As shown in FIG. 8L, such modules 20 may be placed at Fz, PCz, Pz, and Oz locations. The placements shown may be used to target cognitive recovery and sensory integration. Module 20 placements shown may be used to delivery energy or other therapies for broad prefrontal and parietal coverage.

In some examples, the modules 20 may be placed on a portion of the skin above portions of the user (e.g., the gastrointestinal region, the brain, etc.) The modules 20 may be magnetized to portions above the selected bodily region(s). The modules 20 may include or be coupled to one or more sensors 56 that provide a feedback loop from a PBM treatment and/or electrical stimulation treatment. For example, one or more modules 20 may be placed on the external body above the prefrontal cortex (e.g., dorsolateral prefrontal cortex). The modules 20 and/or one or more coupled sensors 56 may provide a treatment at such a location to improve anxiety and/or depression and/or ADHD symptoms. The sensors may detect an improved blood flow or other physiological change and may output such indicators on a display or other output regarding the treatment.

Locations of one or more modules 20 described in any of the embodiments described herein may be initial locations of such modules 20. The locations of the one or more modules 20 may be moved from the initial locations by the user using various methods and devices as described elsewhere herein.

FIG. 8M shows a flow chart of an example closed-loop method of delivering a therapy to the head. One or more steps of the method shown in FIG. 8M may be similar to other methods described herein (e.g. methods shown in FIGS. 6A and 6B). At step 410, one or more initial inputs are entered. Examples of initial inputs and methods and devices to input them are described elsewhere in this specification. At step 412, patient specific module 20 locations are generated. Examples of such locations are disclosed elsewhere in this specification. At step 414, one or more modules 20 are placed at the specific locations on the patient's head. At step 416, initial therapy is delivered at initial parameters. One or more sensors 56 are used to capture data continuously or discretely during or after delivering the initial therapy. At step 420, sensor 56 data and user input(s) (if any) are analyzed. This analysis may be performed to determine one or more of: appropriateness of therapy parameters and module 20 locations. Examples of sensors 56, sensor 56 data, and user input(s) are disclosed elsewhere in this specification. At step 422, one or more therapy parameters and/or module 20 locations are disclosed elsewhere in this specification. At step 424, therapy is delivered with modified parameters. If at step 420, it is determined that the therapy is completed, the therapy stops. If at step 420, it is determined that the therapy is not yet complete, step 434 is performed. At step 434, sensor 56 data and user input(s) (if any) are captured. Thereafter, step 420 is performed.

FIG. 8N shows a flow chart of another example closed-loop method of delivering a therapy to the head. The method shown in FIG. 8N may be similar to the method shown in FIG. 8M. However, the method shown in FIG. 8N has a step 418, wherein therapy and/or sensor 56 data is displayed to the user through a user interface. Examples of therapy data, sensor 56 data, and user interfaces are disclosed elsewhere herein. The user interface may also be used to collect user input(s) (if any) to perform one or more steps of this method.

FIG. 8O shows a flow chart of a method of delivering PBM that uses brain biomarker measurement(s). Such biomarker measurements may be used for sensing or estimating neurovascular activity in the cortex. In some embodiments, diagnostic measurement(s) (e.g. brain biomarker measurement(s), sensor-based measurements, EEG measurements, etc.) may be used to optimize therapeutic PBM in near real-time to the cortex of the brain for treatment of several symptoms and other manifestations of chronic and neurodegenerative disorders. Such symptoms include, but are not limited to: inflammation, constricted or affected blood flow to the cortex, reduced mitochondrial activity and ATP energy production, diminished cognitive capabilities, and changes in signaling activities occurring in the cortex. In some embodiments, biomarkers (e.g. hemoglobin concentration/levels, cytochrome-c-oxidase concentration/levels, etc.) associated with brain conditions may be monitored. Examples of such conditions include, but are not limited to: aging (especially brain and nervous system aging), decline in cognitive abilities, decline in motor skills, Alzheimer's disease, dementia, Parkinson's disease and other neurodegenerative diseases, Major Depressive Disorder (MDD), Attention-deficit/hyperactivity disorder (ADHD), Traumatic Brain Injury (TBI), concussions, Post-traumatic Stress Disorder (PTSD), Obsessive Compulsive Disorder (OCD), Irritable Bowel Syndrome (IBS), Inflammatory Bowel Disease (IBD), eating disorders, anxiety disorders, altered social behavior, physical performance, motivation, schizophrenia, bipolar disorder, gastroparesis, obesity, liver diseases, functional disorders, etc. In some embodiments, neurovascular activity may be measured and therapeutic photobiomodulation may be applied to affect the neurovasculature functioning and achieve a positively impact on the brain condition. The aforementioned conditions may be treated by one or more methods and devices disclosed herein with or without biomarker monitoring. Methods and devices disclosed herein that deliver PBM and other therapies to affect the gut-brain axis, brain functioning or the gut microbiome are particularly suited for treating such conditions, and may be used in combination with other methods and devices, some examples of which are disclosed herein.

Any of the sensing methods and devices disclosed herein may be used to provide PBM and other therapies in a “closed loop” configuration. In such embodiments, sensing or measurement (e.g. measured neurovascular biomarker(s)) may be combined with control of one or more working parameters (emitted wavelength(s), dosing, fluence, pulse width, duty cycle, location, etc.). In some embodiments, the location of PBM modules 20 or other elements may be determined through sensors or by mapping regions of the brain through sensor or other measurements to determine the location(s) of one or more modules 20. Closed loop methods may be used for one or more of: optimization of one or more dose-related working parameters, increasing the life of a battery of module 20, reducing or minimizing time of operation of one or more light emitting elements, reducing discomfort for the user. One or more closed-loop algorithms may be based on a detection-classification scheme. In some embodiments, a three-step process may be used which has the following example steps:
1. Sensing neurovascular activity based on one or more biomarker(s)—(detection step)
2. Conducting one or more computations based on biomarker(s) measurements(s) and relationship(s) to desired outputs—(classification step)
3. Controlling one or more therapy parameters based on the computations.

Such computations may be performed by processor(s) 86, computing device 88, etc. At step 410 in FIG. 8O, diagnostic illumination is applied. This may be applied at a lower power than a subsequent therapeutic illumination. At step 412, one or more sensor(s) 56 are used to measure one or more signals. At step 414, the measured signals are pre-conditioned. This may be performed for one or more of: eliminating/reducing noise, filtering based on bandwidth or frequencies, removing biases, etc. At step 416, data capture, conditioning and digital processing are performed by DAQ xx. In some embodiments, this may be done by one or more of:
1. Capturing, Transforming & Analyzing initial baseline data as reference for ML algorithms,
2. Performing digital calculations e.g. using Modified Beer's Law (e.g. turning raw data to chromophore absorption data)
3. Monitoring trends in chromophore absorption or optical characteristics.

At step 418, baseline biomarker(s) levels are established. At step 420, PBM or other therapies are applied at pre-determined or calculated working parameters (e.g. dosing levels). At step 422, one or more sensor(s) 56 are used to measure one or more signals. At step 424, the current biomarker(s) levels are determined. This may include, for example, updated chromophore absorption data, updated neurovascular activity, etc. At step 426, biomarker(s) levels are analyzed to determine if they are satisfactory. If they are satisfactory, step 420 is performed. If they are not satisfactory, PBM or other therapies are modified accordingly and delivered to the user. This may be done, for example, if the trends in biomarker(s) levels are not as expected. Since there are several working parameters (e.g. power level, duration of therapy, etc.) that can affect chromophore modulation, sequencing and combinatorial approaches may be used to determine the optimum working parameters. An initial part of this method may be performed in an open-loop configuration to established baseline levels. In any of the closed-loop methods and devices disclosed herein, machine learning-based models or other AI models may be used to optimize PBM source parameters and dosing levels to bring about one or more desired actions.

Thus, some method embodiments may involve using sensed data or parameters that are then fed back into a system to influence the therapy e.g. optimize the dosage of the therapy. Such closed-loop systems or feedback-based systems may be designed that are compact and wearable by the user. Various embodiments of modules 20 and wearable devices 10 can be used to position and/or deliver therapy to the optimal region of the body. FIG. 8P shows an example device that is designed to deliver PBM to one or more head regions having hair. Such modules 20 may be designed such that they are attachable to a head-worn wearable device 10. In some embodiments, a module 20 includes one or more sensor 56 (examples disclosed elsewhere in this specification) and one or more light sources to provide light therapy to an area of interest. Hair may be moved away from the optical path by one or more means; examples of which include, but are not limited to: one or more optical guides that displace hair, comb structures to move hair, clips, adhesives, gels, regions on module 20, etc.

FIG. 8Q shows one embodiment of a spectral profile of emitted light from modules 20 along with absorption spectra of specific chromophores. Such spectral profiles may be used in any suitable embodiment herein. Embodiments of optodes that combine illumination and sensing functions may be built using multiple emitter wavelengths that cover an appreciable part of the red/near-infrared spectrum where brain-related chromophores are the most optically active.

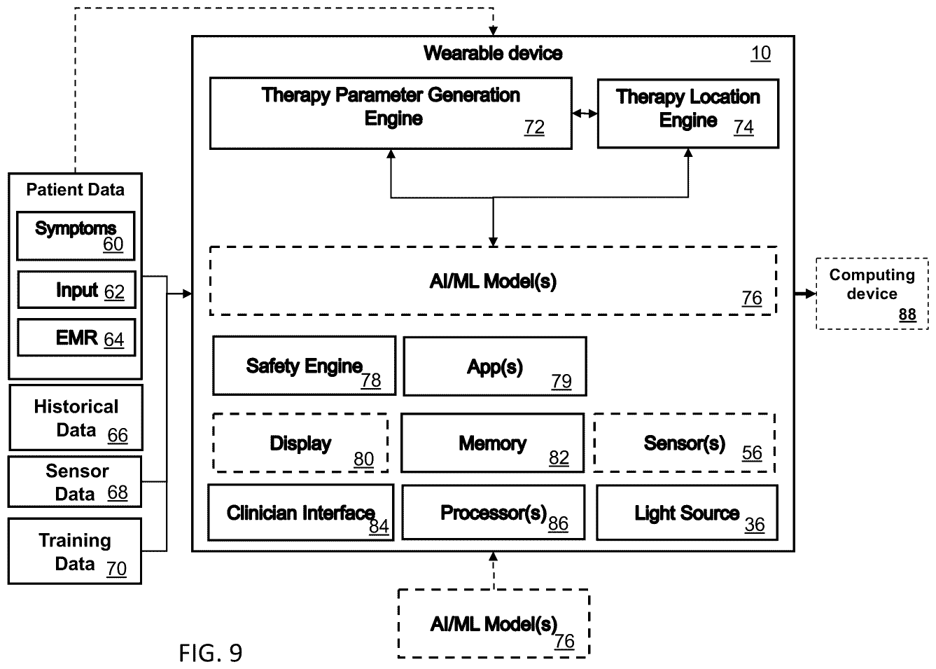

FIG. 9 shows a system diagram of an example computing platform that can be used in any of the embodiments disclosed herein. Wearable device 10 includes one or more user interface(s) for input of one or more data including, but not limited to patient data, historical data 66, sensor data 68, and training data 70. Examples of patient data include, but are not limited to: symptom data 60, patient inputs 62, and electronic medical record (EMR) data 64. Such input data may be automatically transferred to wearable device 10 using one or more user interface(s) or may be manually transferred to wearable device 10 as shown by the dashed line. Wearable device 10 may include one or more AI/ML models 76. The user and/or a clinician may have access to AI/ML models 76. Wearable device 10 also includes a therapy parameter generation engine 72 that communicates with a therapy location engine 74. Therapy parameter generation engine 72 generates one or more therapy parameters of one or more methods described herein. Therapy location engine 74 generates the placement location(s) of one or more modules 20 disclosed herein. Therapy parameter generation engine 72 and therapy location engine 74 may communicate with one or more AI/ML models 76. AI/ML models 76 disclosed herein may be trained using training data that includes, but is not limited to: symptom data 60, patient inputs 62, and electronic medical record (EMR) data 64, etc. Therapy parameter generation engine 72 and therapy location engine 74 may work together to generate any of the outputs disclosed herein including, but not limited to: customized therapy parameter(s) for specific bodily locations of modules 20, customized bodily location(s) for placement of one or more modules 20, etc. For example, therapy parameter generation engine 72 and therapy location engine 74 may work together to generate placement location(s) of one or more modules 20 to deliver therapy to spinal anatomy (as shown In FIGS. 4D and 4E) along with the treatment parameter(s). Such locations(s) and treatment parameter(s) may be evaluated by a clinician or the user before the therapy is delivered. Wearable device 10 also includes a safety engine 78 to check one or more safety aspects of one or more therapies. Wearable device 10 may optionally include a display 80 to display one or more outputs including, but not limited to: module 20 placement information, therapy parameter(s), etc. Wearable device 10 includes a memory 82, one or more sensors 56, a clinical interface 84, one or more processors 86, and one or more light sources 36. Wearable device 10 may optionally communicate with a computing device 88. Examples of computing devices 88 include, but are not limited to: smartphone and other personal communication devices, wearable communication devices, laptops and other electronic devices, etc. In some embodiments, computing device 88 is a part of wearable device 10. Wearable device 10 may communicate with one or more computing devices 88 through a wireless (e.g. Bluetooth, Wi-Fi, etc.) connection or through a wired connection. Data from one or more AI/ML models 76 external to wearable device 10 may be transmitted to wearable device 10.

Apps 79 may interface with one or more components of device 10 and/or AI/ML models 76 and/or device 88. In some embodiments, apps 79 depict output about one or more PBM therapy session. In some embodiments, app(s) 79 may provide graphical content showing a current treatment or therapy dosage, timing, or plan for a user wearing wearable device 10. For example, the apps 79 can provide a graphical plot of one or more values throughout a time period of treatment. This graphical plot and/or view may also mark particular boundaries indicating treatment steps, treatment outcomes, and treatment targets and/or goals. In general, the app 79 may be used by a user of the wearable device 10 to interface with output from AI/ML models 76, optional sensors 56, device 88, or the like. In some embodiments, at least one of the apps 79 may provide a trigger for a sensor (e.g., optional sensor 56) to operate, capture data, send the captured data to an online storage medium (e.g., a cloud server) for ML processing and/or other processing, The app 79 may then display the predicted results on the optional display 80, and/or may display results on another computing device display.

In some embodiments, the system of FIG. 9 may perform a method of delivering photobiomodulation therapy to a user. The method may include having processor 86 execute computer-readable instructions that cause identification of one or more photobiomodulation (PBM) modules configured to deliver individualized PBM therapy to a user via a wearable device. The PBM module(s) may include one or more light-emitting elements, such as LEDs or laser diodes to emit light at specific wavelengths and intensities tailored to a therapeutic protocol, as described elsewhere herein. Identification may be performed by the wearable device's onboard processor, a connected mobile device, or other computing device in communication with processor 86.

The system may identify one or more module attachment sites on the wearable device. These attachment sites may be reversibly coupled to the PBM module(s), enabling secure but detachable mounting. The identification process may include mapping available attachment points, verifying electrical and optical alignment, and confirming that the attachment site supports the specific PBM module type.

Once a PBM module is coupled to a corresponding attachment site, the system receives a connection signal from the wearable device or PBM module. The signal may be generated by a physical sensor (e.g., a magnetic reed switch, mechanical latch sensor, or electrical contact sensor) or via wireless communication between the wearable device and PBM module. This signal serves as confirmation that the PBM module is properly mounted to the wearable device.

The system may receive an input indicating that the wearable device, now with the PBM module attached, is coupled to a body portion of the user. This input may be generated through one or more sensors embedded in the wearable device, such as a proximity sensor, pressure sensor, skin-contact electrode, optical skin detector, or temperature sensor. The detection may occur automatically or be supplemented by user confirmation via a companion app or device interface.

In response to receiving the body-coupling input, the system causes electrical excitation of the PBM modules to commence delivery of photobiomodulation therapy. Electrical excitation parameters, including current, duty cycle, and modulation frequency, in addition to other parameters described herein, may be automatically configured based on the user's personalized therapy protocol. Light output may be continuously monitored and adjusted in real-time to ensure delivery accuracy and adherence to safety limits.

During therapy, the system may monitor operational parameters of the PBM modules, body contact quality, and treatment progress. Feedback may be provided to the user using visual indicators, haptic alerts, or an application interface through the modules 20 and/or the wearable device 10. If a module detachment, poor contact, or unsafe condition is detected, the system may automatically pause or terminate therapy.

AI-based techniques (e.g., those using Generative AI, ML) may be used to create personalized treatment plans with customized therapy parameters and schedules. Such techniques may use one or more inputs, examples of which include, but are not limited to: the user's condition(s), user's input(s), data about the user, evaluation of one or more treatment sessions, stored treatment data, data inputs from cloud-based storage, etc. One or more data inputs described herein may be obtained from an online database which is shared across multiple centers (e.g., treatment sites, research sites, user's homes, etc.). Such database may be open-sourced or otherwise be available to care providers or users.

Similarly, AI-based techniques (e.g., those using Generative AI, ML) may be used for predictive analytics, such that likely treatment outcomes are predicted from one or more inputs.

The ability to collect data from one or more devices 10 disclosed herein and/or ability to remotely adjust the functioning or one or more devices 10 disclosed herein may be used for one or more applications; examples of which include but are not limited to: data-driven R&D, optimization of the therapy, and clinical decision support.

In any of the embodiments disclosed herein, ML-based methods may be used for performing one or more of: data processing (including pre-processing methods such as data denoising to increase quality), feature extraction, segmentation, and feature analysis (which may include clustering and/or classification of one or more features into treatment groups with defined therapy parameters).

One or more devices 10 disclosed herein may be based on Internet of medical things (IoMT) platform, wherein multiple medical devices and applications are connect to healthcare information technology systems through online computer networks. Such devices 10 may include Wi-Fi or Bluetooth or other communication technology. This allows devices 10 to be used for applications including, but not limited to: remote patient monitoring (especially for users with chronic diseases and long-term conditions), tracking patient location, tracking medications or other therapies, collecting data from the patients'wearable devices, and delivering telemedicine at the user's home.

Edge computing may be used to run ML models in device 10 such that one or more applications run sufficiently fast with minimal memory usage. This may be useful in methods where a rapid response is indicated to be performed by device 10 (e.g., real-time adjustment of therapy parameters) or for detecting a sudden change in biometrics. Hybrid approaches combining edge and cloud computing may be used such that certain requests (e.g., requests where a rapid response is indicated to be performed by device 10) are processed on the edge and other requests are handled on the cloud platform.

ML acceleration may be used for hardware and/or software components of device 10. Hardware-accelerated and AI-dedicated cloud services with sufficient processing infrastructure may be used to accelerate ML models. The performance of one or more ML models may be accelerated by running batches of processes in parallel on multiple GPUs for training and/or inference.

Machine Learning Operations (MLOps), a set of practices that help manage the machine learning (ML) lifecycle may be used in any of the embodiments disclosed herein. MLOps may involve tasks such as: experiment tracking, model deployment, model monitoring, and model retraining. MLOps may be used to enable automation and continuous monitoring (to control model and data drift).

“Explainable AI” based methods may be used to offer insights to users and/or care providers on how an ML system handles various sequential processes by placing checkpoints with visual and textual output. One or more Large Language Models (LLMs) may be used to produce a summary or report of one or more outputs of one or more ML systems.

One or more embodiments disclosed herein may use Verifiable AI (vAI), a collection of technologies that help build trust in AI-powered systems by ensuring the integrity of data, algorithms, and hardware. The performance of one or more devices 10 disclosed herein may be verified using the effect(s) of treatment sessions and their adjustability.

In some embodiments, AI-based methods and systems disclosed herein are used to treat and/or manage one or more brain conditions including, but not limited to: Alzheimer's disease and dementia. Such conditions may be treated using personalized treatment plans with customized therapy parameters and schedules.

Any of the embodiments disclosed herein (e.g., embodiments shown in FIG. 1A, FIGS. 4A-4C, FIGS. 5B-5E) may include an array or other arrangement of multiple modules 20 that may be permanently or detachably attached to wearable device 10. The user may deliver targeted therapy using such embodiments by selectively turning some modules 20 on while keeping other modules 20 off. During the use of such embodiments, the user may select one or more parameters including, but not limited to: on/off status of individual modules 20 or groups thereof, energy delivery time, energy dose, type of energy, emitted wavelength(s) and other electromagnetic parameters, etc.

Multiple embodiments of wearable devices 10, modules 20, attachments 90, etc. are disclosed herein. In some embodiments, a user may purchase or otherwise be provided with multiple modules 20 and one or more wearable devices 10, attachments 90, etc. The user can attach one or more modules 20 on one or more wearable devices 10, attachments 90, etc. and use such combinations to deliver PBM and other therapies to multiple regions of their body. One advantage of such a design is that the user can buy modules 20 of the same type and use them to deliver PBM and other therapies sequentially or simultaneously to multiple regions of the body. The working parameters of such modules 20 may be different at one or more bodily regions. The modes of action of such modules 20 may be different at one or more bodily regions. Thus, the location of a module 20 can be changed by the user. Examples of such locations and methods of selecting such locations (e.g. based on organ locations, based on pain/relief felt by the user, based on imaging findings, etc.) are disclosed elsewhere. Thus, multiple organs across a large area (e.g. the abdominal area or the pelvic area, the chest area, etc.) may be targeted simultaneously using multiple modes of action to achieve the desired therapeutic aim(s).

Any of the methods and devices disclosed herein may be used to deliver PBM that activates a drug or enhances the effect of a drug. In such embodiments, one or more drugs may be administered to a patient and PBM is delivered using method and device embodiments disclosed herein.

Any of the methods and devices disclosed herein may be used to diagnose one or more conditions of the user. For example, stored and/or analyzed treatment data may be used to diagnose one or more conditions of the user. Examples of treatment data include, but are not limited to: treatment history, treatment parameters used, treatment adjustments, treatment frequency or other cyclicity, treatment location(s), etc. Such stored and/or analyzed treatment data may be also used for user education and/or increasing user engagement.

Although one or more embodiments disclosed herein are primarily described in terms of wearables worn or otherwise present on the skin, one or more embodiments disclosed herein may be present on other delivery systems. Examples of such systems include, but are not limited to: intravaginal devices, catheter-based systems, and devices inserted into one or more natural or artificially created bodily cavities.

Although the term “patient” is used to describe the person(s) to whom one or more therapies disclosed herein are delivered, this disclosure is not limited to medical therapies alone. Some examples of non-medical uses include, but are not limited to: improving cosmetic outcomes, alleviating aches and pains, improving discomfort or other feelings associated with general well-being, etc.

Any of the therapies disclosed herein may be combined or otherwise used together. For example, a therapy for pelvic pain disclosed herein may be used along with a therapy for back pain disclosed herein. In another example, a therapy for the small or large intestine may be used along with a neck therapy (thus focusing on the gut-brain axis and interactions therein). Such therapies may be delivered simultaneously or at different times. The same modules 20 may be used (e.g., by placing them on separate straps 12) at multiple bodily regions. One or more treatment parameters may be tailored to treat multiple bodily regions.

The examples and illustrations included herein show, by way of illustration and not of limitation, specific embodiments in which the subject matter may be practiced. Other embodiments may be utilized and derived therefrom, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is in fact disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A system for delivering a photobiomodulation therapy, the system comprising:
a wearable device comprising:
an attachment means for securing to a user or a region of clothing worn by the user, and
a first module attachment site; and
a first photobiomodulation module adapted to reversibly attach to the first module attachment site.

2. The system of claim 1, wherein the attachment means comprises one of: a strap, a cap, a belt, a clip, one or more elements that fully or partially encircle a region of a body of the user, one or more wearable garments, one or more elements that are worn over one or more regions of a torso or extremities of the user, one or more wearable elements that wrap around one or more abdominal organs of the user, one or more elements placed over a head of the user, and one or more elements placed over a neck of the user.

3. The system of claim 1, further comprising a second module attachment site and a second photobiomodulation module adapted to reversibly attach to the second module attachment site, the second photobiomodulation module being synchronized according to at least one working parameter of the first photobiomodulation module.

4. The system of claim 1, wherein the first photobiomodulation module is adapted to detect attachment of the first photobiomodulation module to the first module attachment site.

5. The system of claim 1, wherein the wearable device is adapted to be secured to the user at one or more of: a head region, a neck region, a chest region, a belly region, a back region, an abdominal region, one or more arm regions, and one or more leg regions.

6. The system of claim 1, further comprising a charging case adapted for storing and charging the first photobiomodulation module.

7. The system of claim 1, wherein one or more working parameters of the first photobiomodulation module are tunable.

8. The system of claim 1, wherein one or more working parameters of the first photobiomodulation module are programmable using a wireless or a wired connection to the first photobiomodulation module.

9. The system of claim 1, further comprising a sensor on the first photobiomodulation module.

10. The system of claim 1, wherein the attachment means are adapted to be placed on a side of the region of the clothing opposite to the first photobiomodulation module.

11. A computer-implemented method of delivering photobiomodulation therapy to a user, the method comprising:
identifying one or more photobiomodulation modules configured to deliver an individualized photobiomodulation therapy to the user through a wearable device;
identifying one or more module attachment sites configured to reversibly couple to the one or more photobiomodulation modules;
receiving a connection signal indicating that the one or more photobiomodulation modules are coupled to the one or more module attachment sites; and
delivering photobiomodulation using the one or more photobiomodulation modules.

12. A method for delivering a photobiomodulation therapy to a user, the method comprising:
programming a first photobiomodulation module to deliver photobiomodulation at one or more working parameters,
attaching a first wearable device to a first location on a body of the user,
attaching the first photobiomodulation module to a first location of the first wearable device,
delivering photobiomodulation through the first photobiomodulation module at the one or more working parameters.

13. The method of claim 12, wherein one or more working parameters comprise one or more of: an on/off status of a therapy, an emitted wavelength(s), a wavelength distribution and/or bandwidth, a power output, an irradiance, a fluence, a pulse frequency, a pulse width, a duty cycle, a coherence, a beam divergence, a polarization, a spot size, a beam diameter, a beam profile, a delivery mode, an incidence angle, a number and/or type of emitters that are operational, a cooling mechanism(s), a therapy duration, an on/off status of a display, information displayed on a screen, an on/off status of a user interface, a type of information displayed on a user interface, and for one or more sensors in use: an on/off status, a type, working parameters, a sampling frequency, an energy output, and an energy consumption.

14. The method of claim 12, further comprising adjusting at least one of the one or more working parameters.

15. The method of claim 14, wherein the adjusting of the one or more working parameters is performed by one or more of:
a manual process through a user interface,
an automatic process performed by the first photobiomodulation module,
an automatic process performed by an application loaded on an electronic device in communication with the first photobiomodulation modules or one or more sensors in use during the photobiomodulation therapy,
responsive to detecting a magnetic attachment to the first photobiomodulation modules, and using data from the one or more sensors.

16. The method of claim 12, further comprising attaching the first photobiomodulation module to a second location of the first wearable device.

17. The method of claim 12, further comprising attaching a second photobiomodulation module to a second location of the first wearable device and delivering photobiomodulation using the second photobiomodulation module.

18. The method of claim 17, wherein at least one working parameter of the second photobiomodulation module is different than a working parameter of the first photobiomodulation module.

19. The method of claim 12, further comprising attaching a second photobiomodulation module to a first location of a second wearable device and delivering photobiomodulation using the second photobiomodulation module.

20. The method of claim 17, wherein the first photobiomodulation module delivers photobiomodulation to one or more of: a brain region, a skin region, a scalp region, one or more hair follicles, on or more eyes, regions of an oral cavity, a jaw region, a neck region, a spine region, a shoulder region, an elbow region, a wrist region, one or more hands, one or more feet, one or more fingers, a chest region, a heart region, one or more lung regions, a liver region, one or more kidney regions, a bladder regions, a uterus region, a prostate region, a pelvic floor region, a colon region, a rectum region, a perineum region, one or more reproductive organs, one or more skeletal muscles, one or more cardiac muscles, one or more joints, one or more tendons, and one or more bones.

21. The method of claim 12, further comprising:
providing a second photobiomodulation module, wherein the method further comprises synchronizing at least one working parameter of the first photobiomodulation module to at least one working parameter of the second photobiomodulation module.
